(19) United States
(12) Patent Application Publication
(10) Pub. No.: US 2002/0119961 A1
(43) Pub. Date: Aug. 29, 2002

(54) Bridged piperazine derivatives

(75) Inventors: Laura C. Blumberg, Waterford, CT (US); Matthew F. Brown, Stonington, CT (US); Ronald P. Gladue, Stonington, CT (US); Christopher S. Poss, North Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 09/972,177

(22) Filed: Oct. 5, 2001

Related U.S. Application Data
(60) Provisional application No. 60/241,804, filed on Oct. 19, 2000.

(51) Int. Cl. C07D 471/06; A61K 31/4748; A61K 31/47; A61K 31/397

(52) U.S. Cl. 514/210.16; 514/285; 514/286; 514/410; 546/63; 546/74; 548/424; 548/950

(57) ABSTRACT

A compound of the formula

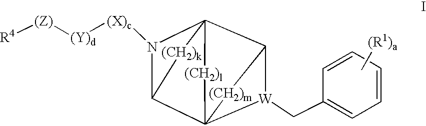

or the pharmaceutically acceptable salt thereof; wherein a, c, d, k, l, m, W, X, Y Z, R¹, and R⁴ are as defined, and useful to treat inflammation and other immune disorders.

### BACKGROUND OF THE INVENTION

The present invention relates to novel piperazine derivatives, methods of use and pharmaceutical compositions containing them.

The compounds of the invention are potent and selective inhibitors of chemokines binding to the receptor CCR1 found on inflammatory and immunomodulatory cells (preferably leukocytes and lymphocytes). The CCR1 receptor is also sometimes referred to as the CC-CKR¹ receptor. These compounds also inhibit MIP-1α (and the related chemokines shown to interact with CCR1 (e.g., RANTES, HCC-1, MCP-2 and MCP-3)) induced chemotaxis of THP-1 cells and human leukocytes and are potentially useful for the treatment or prevention of autoimmune diseases (such as rheumatoid arthritis, type I diabetes (recent onset), lupus, inflammatory bowel disease, optic neuritis, psoriasis, multiple sclerosis, polymyalgia rheumatica, uveitis, and vasculitis), acute and chronic inflammatory conditions (such as osteoarthritis, adult Respiratory Distress Syndrome, Respiratory Distress Syndrome of infancy, ischemia reperfusion injury, glomerulonephritis, and chronic obstructive pulmonary disease (COPD)), allergic conditions (such as asthma and atopic dermatitis), inflammation associated with infection (such as viral inflammation (including influenza, hepatitis and Guillian-Barre), chronic bronchitis, tissue, cell and solid organ transplant rejection (including xeno transplantations) (chronic and acute), atherosclerosis, restenosis, HIV infectivity (co-receptor usage), and granulomatous diseases (including sarcoidosis, leprosy and tuberculosis) and sequelae associated with certain cancers such as multiple myeloma. Compounds in this series may also limit the production of cytokines at inflammatory sites, including but not limited to TNF and IL-1, as a consequence of decreasing cell infiltration, providing benefit for diseases linked to TNF and IL-1, including congestive heart failure, pulmonary emphysema or dyspnea associated therewith, emphysema; HIV-1, HIV-2, HIV-3; cytomegalovirus (CMV), adenoviruses, Herpes viruses (Herpes zoster and Herpes simplex). They may also provide benefit for the sequelae associated with infection where such infection induces production of detrimental inflammatory cytokines such as TNF e.g, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease, septic shock, cancer, trauma, and malaria, etc.

MIP-1α and RANTES are soluble chemotactic peptides (chemokines) which are produced by inflammatory cells, in particular CD8+ lymphocytes, polymorphonuclear leukocytes (PMNs) and macrophages, J. Biol. Chem., 270 (30) 29671-29675 (1995). These chemokines act by inducing the migration and activation of key inflammatory and immunomodulatory cells. Elevated levels of chemokines have been found in the synovial fluid of rheumatoid arthritis patients, chronic and acute rejecting tissue from transplant patients and in the nasal secretions of allergic rhinitis patients following allergen exposure (Teran, et al., J. Immunol., 1806-1812 (1996), and Kuna et al., J. Allergy Clin. Immunol. 321 (1994)). Antibodies which interfere with the chemokine/receptor interaction by neutralizing MIP1α or gene disruption have provided direct evidence for the role of MIP-1 a and RANTES in disease by limiting the recruitment of monocytes and CD8+lymphocytes (Smith et al., J. Immunol, 153, 4704 (1994) and Cook et al., Science, 269, 1583 (1995)). Together this data demonstrates that CCR1 receptor antagonists would potentially be an effective treatment of several immune based diseases. The compounds described within are potent and selective antagonists of the CCR1 receptor.

### SUMMARY OF THE INVENTION

The present invention relates to a compound of the formula

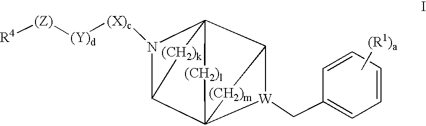

or pharmaceutically acceptable salts and pro-drugs thereof; wherein

a is 1,2,3,4 or 5;

c is 0 or 1;

d is 1, 2, 3, 4 or 5;

k is 0, 1, 2, 3 or 4; l is 0, 1, 2, 3 or 4; m is 0, 1, 2, 3, or 4; k, l and m cannot all be 0 and if m and/or k are not 0, then l must be 0.

W is CH or N;

X is C(O), C(S) or CH₂;

Y is CH₂;

Z is oxygen, NR⁹ or CR¹¹R¹²;

each R¹ is independently selected from hydrogen, hydroxy, hydroxysulfonyl, halo, (C₁-C₆)alkyl, mercapto, mercapto(C₁-C₆)alkyl, (C₁-C₆)alkylthio, (C₁-C₆)alkylsulfinyl, (C₁-C₆)alkylsufonyl, (C₁-C₅)alkylthio(C₁-C₆)alkyl, (C₁-C₆)alkylsulfinyl(C₁-C₆)alkyl, (C₁-C₆)alkylsulfonyl(C₁-C₆)alkyl, (C₁-C₆)alkoxy, (C₆-C₁₀)aryloxy, halo(C₁-C₆)alkyl, trifluoromethyl, formyl, formyl(C₁-C₆)alkyl, nitro, nitroso, cyano, (C₆-C₁₀)aryl(C₁-C₆)alkoxy, halo(C₁-C₆)alkoxy, trifluoromethoxy, (C₃-C₇)cycloalkyl, (C₃-C₇)cycloalkyl(C₁-C₆)alkyl, hydroxy(C₃-C₇)cycloalkyl(C₁-C₆)alkyl, (C₃-C₇)cycloalkylamino, (C₃-C₇)cycloalkylamino(C₁-C₆)alkyl, ((C₃-C₇)cycloalkyl)((C₁-C₆)alkyl)amino, ((C₃-C₇)cycloalkyl(C₁-C₆)alkyl)amino(C₁-C₆)alkyl, cyano(C₁-C₆)alkyl, (C₂-C₇)alkenyl, (C₂-C₇)alkynyl, (C₆-C₁₀)aryl, (C₆-C₁₀)aryl(C₁-C₆)alkyl, (C₆-C₁₀)aryl(C₂-C₆)alkenyl, hydroxy(C₁-C₆)alkyl, hydroxy(C₆-C₁₀)aryl(C₁-C₆)alkyl, hydroxy(C₁-C₆)alkylthio(C₁-C₆)alkyl, hydroxy(C₂-C₆)alkenyl, hydroxy(C₂-C₆)alkynyl, (C₁-C₆)alkoxy(C₁-C₆)alkyl, (C₁-C₆)alkoxy(C₆-C₁₀)aryl(C₁-C₆)alkyl, (C₆-C₁₀)aryloxy(C₁-C₆)alkyl, (C₆-C₁₀)aryl(C₁-C₆)alkoxy(C₁-C₆)alkyl, amino, (C₁-C₆)alkylamino, ((C₁-C₆)alkyl)₂amino, (C₆-C₁₀)arylamino, (C₆-C₁₀)aryl(C₁-C₆)alkylamino, amino(C₁-C₆)alkyl, (C₁-C₆)alkylamino(C₁-C₆)alkyl, ((C₁-C₆)alkyl)₂amino(C₁-C₆)alkyl, hydroxy(C₁-C₆)alkylamino(C₁-C₆)alkyl, (C₆-C₁₀)arylamino(C₁-C₆)alkyl, (C₆-C₁₀)aryl (C₁-C₆)alkylamino(C₁-C₆)alkyl, (C₁-C₆)alkylcarbonylamino, ((C₁-C₆)alkylcarbonyl)((C₁-C₆)alkyl)amino, (C₁-C₆)alkylcarbonylamino(C₁-C₆)alkyl, ((C₁-C₆)alkylcarbonyl)((C₁-C₆)alkyl)amino(C₁-C₆)alkyl, (C₁-C₆)alkoxycarbonylamino, (C₁-C₆)alkoxycarbonyl)(C₁-C₆)alkylamino, (C₁-C₆)alkoxycarbonylamino(C₁-C₆)alkyl, (C₁-C₆)alkoxycarbnony)((C₁-C₆)alkyl)amino(C₁-Cr)alkyl, carboxy, (C₁-C₆)alkoxycarbonyl, (C₆-C₁₀)aryl(C₁-C₆)alkoxycarbonyl, (C₁-C₆)alkylcarbonyl, (C₁-C₆)alkylcarbonyl(C₁-C₆)alkyl, (C₆-C₁₀)arylcarbonyl, (C₆-C₁₀)arylcarbonyl(C₁-C₆)alkyl, (C₆-C₁₀)aryl(C₁-C₆)alkylcarbonyl, (C₆-C₁₀)aryl(C₁-C₆)alkycarbonyl(C₁-C₆)alkyl, carboxy(C₁-C₆)alkyl, (C₁-C₆)alkoxycarbonyl(C₁-C₆)alkyl, (C₆-C₁₀)aryl(C₁-C₆)alkoxycarbonyl(C₁-C₆)alkyl, (C₁-C₆)alkoxy(C₁-C₆)alkylcarbonyloxy(C₁-C₆)alkyl, aminocarbonyl, (C₁-C₆)alkylaminocarbonyl, ((C₁-C₆)alkyl)₂aminocarbonyl, (C₆-C₁₀)arylaminocarbonyl, (C₆-C₁₀)aryl(C₁-C₆)alkylaminocarbonyl, aminocarbonyl(C₁-C₆)alkyl, (C₁-C₆)alkylaminocarbonyl(C₁-C₆)alkyl, ((C₁-C₆)alkyl)₂aminocarbonyl(C₁-C₆)alkyl, (C₆-C₁₀)arylaminocarbonyl(C₁-C₆)alkyl, (C₁-C₆)alkylaminocarbonyl(C₁-C₆)alkyl, amidino, guanidino, ureido, (C₁-C₆)alkylureido, ((C₁-C₆)alkyl)₂ureido, ureido(C₁-C₆)alkyl, (C₁-C₆)alkylureido(C₁-C₆)alkyl, ((C₁-C₆)alkyl)₂ureido(C₁-C₆)alkyl, (C₂-C₉)heterocycloalkyl, (C₂-C₉)heteroaryl, (C₂-C₉)heterocycloalkyl(C₁-C₆)alkyl and (C₂-C₉)heteroaryl(C₁-C₆)alkyl;

R⁴ is (R⁵Q_{q})_{f}(C₆-C₁₀)aryl, (R⁵Q_{q})_{f}(C₃-C₁₀)cycloalkyl, (R⁵Q_{q})_{f}(C₂-C₉)heteroaryl, (R⁵Q_{q})_{f}(C₂-C₉)heterocycloalkyl,

wherein f is 0, 1, 2, 3, 4 or 5;

Q is (C₁-C₆)alkyl;

q is 0 or 1;

R⁵ is independently selected from (C₂-C₉)heterocycloalkylcarbonyl, (C₂-C₉)heteroarylcarbonyl, (C₂-C₉)heteroaryl(C₁-C₆)alkylaminocarbonyl, (C₂-C₉)heteroarylaminocarbonyl, (C₂-C₉)heterocycloalkyl(C₁-C₆)alkylaminocarbonyl, (C₁-C₆)alkylsulfonylaminocarbonyl, (C₁-C₆)alkylsuffonylamino(C₁-C₆)alkylaminocarbonyl, ureido(C₁-C₆)alkylaminocarbonyl, (C₁-C₆)alkylureido(C₁-C₆)alkylaminocarbonyl, ((C₁-C₆)alkyl)₂ureido(C₁-C₆)alkylaminocarbonyl, halo(C₁-C₆)alkylaminocarbonyl, (C₁-C₆)alkylcarbonylamino(C₁-C₆)alkylaminocarbonyl, hydroxy(C₁-C₆)alkylaminocarbonyl, aminosulfonyl(C₁-C₆)alkylaminocarbonyl, carboxy(C₁-C₆)alkylaminocarbonyl, (C₁-C₆)alkylaminosulfonyl(C₁-C₆)alkylaminocarbonyl, amino(C₁-C₆)alkylcarbonylamino, (C₁-C₆)alkylamino(C₁-C₅)alkylcarbonylamino, carboxy(C₁-C₆)alkylcarbonylamino, carboxy(C₁-C₆)alkoxycarbonylamino, ((C₁-C₆)alkyl)₂amino(C₁-C₆)alkylcarbonylamino, acetylamino(C₁-C₆)alkylcarbonylamino, (acetyl)((C₁-C₆)alkyl)amino(C₁-C₆)alkylcarbonylamino, (C₁-C₆)alkylsulfonylamino(C₁-C₆)alkylcarbonylamino, cyanoguan idino(C₁-C₆)alkylcarbonylamino, (C₁-C₆)alkylcyanoguanidino(C₁-C₆)alkylcarbonylamino, ((C₁-C₆)alkyl)₂cyanoguanidino(C₁-C₆)alkylcarbonylamino, aminocarbonyl(C₁-C₆)alkylcarbonylamino, aminocarbonylamino(C₁-C₆)alkylcarbonylamino, (C₁-C₆)alkylaminocarbonylamino(C₁-C₆)alkylcarbonylamino, ((C₁-C₆)alkyl)₂aminocarbonylamino(C₁-C₆)alkylcarbonylamino, (C₂-C₉)heteroaryl(C₁-C₆)alkylcarbonylamino, (C₂-C₉)heterocycloalkyl(C₁-C₆)alkylcarbonylamino, aminosulfonyl(C₁-C₆)alkylcarbonylamino, hydroxy(C₁-C₆)alkylureido, amino(C₁-C₆)alkylureido, (C₁-C₆)alkylamino(C₁-C₆)alkylureido, ((C₁-C₆)alkyl)₂amino(C₁-C₆)alkylureido, (C₂-C₉)heterocycloalkyl(C₁-C₆)alkylureido, (C₂-C₉)heteroarylureido, (C₂-C₉)heteroaryl(C₁-C₆)alkylureido, (C₁-Cr)alkylsulfonylureido, aminosulfonyl(C₁-C₆)alkylureido, aminocarbonyl(C₁-C₆)alkylureido, (C₁-C₆)alkylaminocarbonyl(C₁-C₆)alkylureido, ((C₁-C₆)alkyl)₂aminocarbonyl(C₁-C₆)alkylureido, acetylamino(C₁-C₆)alkylureido, (acetyl)((C₁-C₆)alkyl)amino(C₁-C₆)alkylureido, carboxy(C₁-C₆)alkylureido, halo(C₁-C₆)alkylsulfonylamino, amino(C₁-C₆)alkylsulfonylamino, (C₁-C₆)alkylamino(C₁-C₆)alkylsulfonylamino, ((C₁-C₆)alkyl)₂amino(C₁-C₆)alkylsulfonylamino, acetylamino(C₁-C₆)alkylsulfonylamino (acetyl)((C₁-C₆)alkyl)amino(C₁-C₆)alkylsulfonylamino, ureido(C₁-C₆)alkylsulfonylamino, (C₁-C₆)alkylureido(C₁-C₆)alkylsulfonylamino, ((C₁-.C₆)alkyl)₂ureido(C₁-C₆)alkylsulfonylamino, (C₁-C₆)alkylsulfonylamino(C₁-C₆)alkylsulfonylamino, cyanoguanidino(C₁-C₆)alkylsulfonylamino, carboxy(C₁-C₆)alkylsulfonylamino, (C₁-C₆)alkylcyanoguanidino(C₁-C₆)alkylsulfonylamino, ((C₁-C₆)alkyl)₂cyanoguanidino(C₁-C₆)alkylsulfonylamino, aminocarbonyl(C₁-C₆)alkylsulfonylamino, (C₁-C₆)alkoxycarbonylamino(C₁-C₆)alkylsulfonylamino, aminosulfonylaminocarbonyl, (C₁-C₆)alkylaminosulfonylaminocarbonyl, ((C₁-C₆)alkyl)₂aminosulfonylaminocarbonyl, (C₆-C₁₀)arylsulfonyl, (C₁-C₆)alkylaminosulfonylamino, ((C₁-C₆)alkyl)₂aminosulfonylamino, aminocarbonyl(C₁-C₆)alkylamino(C₁-C₆)alkylsulfonylamino, (C₂-C₉)heterocycloalkyloxycarbonylamino(C₁-C₆)alkylsulfonylamino, (C₂-C₉)heteroaryloxycarbonylamino(C₁-C₆)alkylsulfonylamino, cyanoguanidino, (C₁-C₆)alkylcyanoguanidino, ((C₁-C₆)alkyl)₂cyanoguanidino, (C₂-C₉)heterocycloalkylcyanoguanidino, (C₂-C₉)heterocycloalkyl(C₁-C₆)alkylcyanoguanidino, (C₂-C₉)heteroaryl(C₁-C)alkylcyanoguanidino, amino(C₁-C₆)alkylcyanoguanidino, (C₁-C₆)alkylamino(C₁-C₆)alkylcyanoguanidino, ((C₁-C₆)alkyl)₂amino(C₁-C₆)alkylcyanoguanidino, aminocarbonyl(C₁-C₆)alkylcyanoguanidino, carboxy(C₁-C₆)alkylcyanoguanidino; (C₁-C₆)alkylaminocarbonyl(C₁-C₆)alkylcyanoguanidino, ((C₁-C₆)alkyl)₂aminocarbonyl(C₁-C₆)alkylcyanoguanidino, hydroxy(C₁-C₆)alkylamino, aminocarbonyl(C₁-C₆)alkylamino, carboxy(C₁-C₆)alkylamino, (C₁-C₆)alkylsulfonylamino(C₁-C₆)alkylamino, (C₁-C₆)alkoxycarbonylamino(C₁-C₆)alkylamino, aminosulfonyl(C₁-C₆)alkylamino, (C₂-C₉)heteroaryl(C₁-C₆)alkylamino, acetylamino(C₁-C₆)alkylamino, (acetyl)((C₁-C₆)alkyl)amino(C₁-C₆)alkylamino, (C₂-C₉)heterocycloalkyl(C₁-C₆)alkylamino, ((C₁-C₆)alkyl)₂amino(C₁-C₆)alkylamino, (C₁-C₆)alkylamino(C₁-C₆)alkylamino,(C₁-C₆)alkoxy(C₁-C₆)alkylamino, (C₁-C₆)alkoxycarbonyl(C₁-C₆)alkylamino, cyano(C₁-C₆)alkylamino, (C₂-C₉)heterocycloalkyloxycarbonylamino(C₁-C₆)alkylamino, (C₂-C₉)heteroaryloxycarbonylamino(C₁-C₆)alkylamino, cyanoguanidino(C₁-C₆)alkylamino, (C₁-C₆)alkylcyanoguanidino(C₁-C₆)alkylamino, ((C₁-C₆)alkyl)₂cyanoguanidino(C₁-C₆)alkylamino, ureido(C₁-C₆)alkylamino, (C₁-C₆)alkylureido(C₁-C₆)alkylamino, ((C₁-C₆)alkyl)₂ureido(C₁-C₆)alkylamino, aminocarbonyloxy(C₁-C₆)alkylamino, hydroxy(C₁-C₆)alkylcarbonylamino, (C₁-Cr)alkylaminocarbonyl(C₁-C₆)alkylcarbonylamino, ((C₁-C₆)alkyl)₂aminocarbonyl(C₁-C₆)alkylcarbonylamino, (C₁-C₆)alkoxycarbonylamino(C₁-C₆)alkylcarbonylamino, aminosulfonyl(C₁-C₆)alkylcarbonylamino, hydroxy(C₁-C₆)alkylamino(C₁-C₆)alkylcarbonylamino, ((C₁-C₆)alkyl)₂amino(C₁-C₆)alkylamino(C₁-C₆)alkylcarbonylamino (C₁-C₆)alkylamino(C₁-C₆)alkylamino(C₁-C₆)alkylcarbonylamino, amino(C₁-C₆)alkylamino(C₁-C₆)alkylcarbonylamino, (C₁-C₆)alkoxy(C₁-C₆)alkylamino(C₁-C₆)alkylcarbonylamino, (C₂-C₉)heterocycloalkyloxycarbonylamino, (C₂-C₉)heteroarylcarbonylamino(C₁-C₆)alkylcarbonylamino, (C₂-C₉)heteroarylcarbonylamino, (C₂-C₉)heterocycloalkylcarbonylamino, (C₂-C₉)heteroaryl(C₁-C₆)alkylcarbonylamino, (C₂-C₉)heterocycloalkyl(C₁-C₆)alkylcarbonylamino, (C₂-C₉)heterocycloalkylcarbonylamino(C₁-C₆)alkylcarbonylamino, cyano(C₁-C₆)alkylcarbonylamino, (C₁-C₆)alkylsulfonylamino(C₁-C₆)alkylaminocarbonylamino, (C₁-C₆)alkoxycarbonylamino(C₁-C₆)alkylaminocarbonylamino, (C₂-C₉)heterocycloalkyloxycarbonylamino(C₁-C₆)alkylaminocarbonylamino, (C₂-C₉)heteroaryloxycarbonylamino(C₁-C₆)alkylaminocarbonylaminol, ureido(C₁-C₆)alkylureido, (C₁-C₆)alkylureido(C₁-C₆)alkylureido, ((C₁-C₆)alkyl)₂ureido(C₁-C₆)alkylureido, cyanoguanidino(C₁-C₆)alkylureido, (C₂-C₉)heteroaryl(cyanoguanidino), aminosulfonyl, amino(C₁-C₆)alkylsulfonyl, (C₁-C₆)alkylamino(C₁-C₆)alkylsulfonyl, ((C₁-C₆)alkyl)₂amino(C₁-C₆)alkylsulfonyl, (C₁-C₆)alkylaminosulfonyl, ((C₁-C₆)alkyl)₂aminosulfonyl, (C₂-C₉)heterocycloalkylsulfonyl, amino(C₁-C₆)alkylaminosulfonyl, (C₁-C₆)alkylamino(C₁-C₆)alkylaminosulfonyl, ((C₁-C₆)alkyl)₂amino(C₁-C₆)alkylaminosulfonyl, (C₂-C₉)heteroarylaminosulfonyl, hydroxy(C₁-C₆)alkylaminosulfonyl, (C₁-C₆)alkoxy(C₁-C₆)alkylaminosulfonyl, ureido(C₁-C₆)alkylaminosulfonyl, (C₁-C₆)alkylureido(C₁-C₆)alkylaminosulfonyl, ((C₁-C₆)alkyl)₂ureido(C₁-C₆)alkylaminosulfonyl, (C₁-Cc)alkylsulfonylamino(C₁-C₆)alkylaminosulfonyl, (C₁-C₆)alkoxycarbonylamino(C₁-C₆)alkylaminosulfonyl, (C₂-C₉)heterocycloalkyloxycarbonylamino(C₁-C₆)alkylaminosulfonyl, (C₂-C₉)heteroaryloxycarbonylamino(C₁-C₆)alkylaminosuffonyl, aminocarbonyl(C₁-C₆)alkylaminosulfonyl, cyanoguanidino(C₁-C₆)alkylaminosulfonyl, (C₂-C₉)heteroarylaminosulfonyl, (C₂-C₉)heteroaryl(C₁-C₆)alkylaminosulfonyl, (C₂-C₉)heterocycloalkylaminosulfonyl, (C₁-C₆)alkylcarbonylaminosulfonyl, halo(C₁-C₆)alkylcarbonylaminosulfonyl, (C₁-C₆)alkoxycarbonylaminosulfonyl, ureidosulfonyl, (C₁-C₆)alkylureidosulfonyl, ((C₁-C₆)alkyl)₂ureidosulfonyl, hydrogen, hydroxy, hydroxysulfonyl, halo, mercapto, (C₁-C₆)alkylthio, (C₁-C₆)alkylsulfinyl, (C₁-C₆)alkylsulfonyl, carboxy(C₁-C₆)alkylsulfonyl, (C₆-C₁₀)arylsulfonyl, (C₂-C₉)heteroarylsulfonyl, (C₁-C₆)alkoxy, hydroxy(C₁-C₆)alkoxy, (C₆-C₁₀)aryloxy, trifluoro(C₁-C₆)alkyl, formyl, nitro, nitroso, cyano, halo(C₁-C₆)alkoxy, trifluoro(C₁-C₆)alkoxy, amino(C₁-C₆)alkoxy, (C₃-C₁₀)cycloalkylhydroxy(C₃-C₁₀)cycloalkyl (C₃-C₁₀)cycloalkylamino(C₂-C₆)alkenyl, (C₂-C₆)alkynyl, (C₆-C₁₀)aryl, (C₆-C₁₀)aryl(C₂-C₆)alkenyl, hydroxy(C₆-C₁o)aryl, ((C₁-C₆)alkylamino)(C₆-C₁₀)aryl, hydroxy(C₁-C₆)alkylthio, hydroxy(C₂-C₆)alkenyl, hydroxy(C₂-Ca)alkynyl, (C₁-C₆)alkoxy(C₆-C₁₀)aryl, (C₆-C₁₀)aryl(C₁-C₆)alkoxy, amino, (C₁-C₆)alkylamino, ((C₁-C₆)alkyl)₂amino, (C₆-C₁₀)arylamino, (C₆-C₁₀)aryl(C₁-C₆)alkylamino, amino(C₁-C₆)alkylamino, (C₂-C₉)heterocycloalkylamino, (C₂-C₉)heteroarylamino, (C₂-C₉)heteroaryl(C₁-C₆)alkylamino, (C₂-C₉)heterocycloalkyl(C₁-C₆)alkylamino, (C₃-C₁₀)cycloalkyl(C₁-C₆)alkyl)amino, (C₁-C₆)alkylcarbonylamino, (C₁-C₆)alkoxycarbonylamino, (C₂-C₆)alkenylcarbonylamino, (C₃-C₁₀)cycloalkylcarbonylamino, (C₆-C₁₀)arylcarbonylamino, (C₂-C₉)heterocycloalkylcarbonylamino, (C₂-C₉)heteroaryloxycarbonylamino, (C₂-C₉)heterocycloalkoxycarbonylamino, halo(C₁-C₆)alkylcarbonylamino, (C₁-C₆)alkoxy(C₁-C₆)alkylcarbonylamino, (C₁-C₆)alkoxycarbonyl(C₁-C₆)alkylcarbonylamino, ((C₁-C₆)alkylcarbonyl)((C₁-C₆)alkyl)amino, ((C₁-C₆)alkoxycarbonyl)((C₁-C₆)alkyl)amino, (C₁-C₆)alkylsulfonylamino, ((C₁-C₆)alkylcarbonyl)((C₁-C₆)alkyl)amino, (C₃-C₁₀)cycloalkyl(C₁-C₆)alkyl)amino, ((C₁-C₆)alkylsulfonyl)((C₁-C₆)alkyl)amino, (C₂-C₉)heteroarylsulfonylamino, (C₆-C₁₀)arylsulfonylamino, ((C₆-C₁₀)arylsulfonyl)((C₁-C₆)alkyl)amino, carboxy, (C₁-C₆)alkoxycarbonyl, (C₆-C₁₀)aryl(C₁-C₆)alkoxycarbonyl, (C₁-C₆)alkylcarbonyl, carboxy(C₁-C₆)alkylcarbonyl, amino(C₁-C₆)alkylcarbonyl, (C₁-C₆)alkylamino(C₁-C₆)alkylcarbonyl, ((C₁-C₆)alkyl)₂amino(C₁-C₆)alkylcarbonyl, (C₆-C₁₀)arylcarbonyl, (C₂-C₉)heteroaryl(C₁-C₆)alkylcarbonyl, (C₆-C₁₀)aryl(C₁-C₆)alkylcarbonyl, hydroxy(C₁-C₆)alkoxycarbonyl, (C₁-C₆)alkoxy(C₁-C₆)alkylcarbonyloxy, ((C₁-C₆)alkyl)₂aminocarbonyloxyaminocarbonyl, hydroxyaminocarbonyl, (C₁-C₆)alkylaminocarbonyl, ((C₁-C₆)alkyl)₂aminocarbonyl, (C₆-C₁₀)arylaminocarbonyl, (C₆-C₁₀)aryl(C₁-C₆)alkylaminocarbonyl, (aminocarbonyl(C₁-C₆)alkylaminocarbonyl, ((C₁-C₆)alkylaminocarbonyl(C₁-C₆)alkylaminocarbonyl, (carboxy(C₁-C₆)alkyl)aminocarbonyl, ((C₁-C₆)alkoxycarbonyl(C₁-C₆)alkylaminocarbonyl, (amino(C₁-C₆)alkyl)aminocarbonyl, (hydroxy(C₁-C₆)alkylaminocarbonyiamidino, hydroxyamidino, guanidino, ureido, (C₁-C₆)alkylureido, (C₆-C₁₀)arylureido, ((C₆-C₁₀)aryl)₂ureido, (C₆-C₁₀)aryl(C₁-C₆)alkylureido, halo(C₁-C₆)alkylureido, ((C₁-C₆)alkyl)((C₆-C₁₀)aryl)ureido, ((C₁-C₆)alkyl)₂ureido, halo(C₁-C₆)alkylcarbonylureido, (halo(C₁-C₆)alkyl)((C₁-C₆)alkyl)ureido, ((C₁-C₆)alkoxycarbonyl(C₁-C₆)alkyl)ureido, glycinamido, (C₁-C₆)alkylglycinamido, aminocarbonylglycinamido, (C₁-C₆)alkoxy(C₁-C₆)alkylcarbonylglycinamido, (aminocarbonyl)((C₁-C₆)alkyl)glycinamido, ((C₁-C₆)alkoxycarbonyl(C₁-C₆)alkylcarbonyl)((C₁-C₆)alkyl)glycinamido, ((C₁-C₆)alkoxycarbonylamino(C₁-C₆)alkylcarbonyl)glycinamido, (C₆-C₁₀)arylcarbonylglycinamido, ((C₆-C₁₀)arylcarbonyl)((C₁-C₆)alkyl)glycinamido, ((C₆-C₁₀)aryl(C₁-C₆)alkylaminocarbonyl)glycinamido, (C₆-C₁o)aryl(C₁-C₆)alkylaminocarbonyl)((C₁-C₆)alkyl)glycinamido, (C₆-C₁₀)arylaminocarbonylglycinamido, ((C₆-C₁₀)arylaminocarbonyl)((C₁-C₆)alkyl)glycinamido, alaninamido, (C₁-C₆)alkylalaninamido, (C₂-C₉)heteroaryl, amino(C₂-C₉)heteroaryl, (C₁-C₆)alkylamino(C₂-C₉)heteroaryl, ((C₁-C₆)alkyl)₂amino(C₂-C₉)heteroaryl, (C₂-C₉)heteroaryloxy, (C₂-C₉)heterocycloalkyl, carboxy(C₁-C₆)alkoxy, (C₁-C₆)alkylsulfonylaminocarbonyl(C₁-C₆)alkoxy, (C₁-C₆)alkylsulfonylamino(C₁-C₆)alkoxy, (C₂-C₉)heteroaryl(C₁-C₆)alkoxy, carboxy(C₁-C₆)alkylamino(C₂-C₆)alkoxy, amino(C₂-C₆)alkoxy, (aminocarbonyl)(hydroxy)amino, (C₁-C₆)alkylamino(C₂-C₆)alkoxy, ((C₁-C₆)alkyl)₂amino(C₂-Ca)alkoxy, (C₁-C₆)alkylcarbonylamino(C₂-C₆)alkoxy, aminocarbonylamino(C₂-C₆)alkoxy, (C₁-C₆)alkylaminocarbonylamino(C₂-C₆)alkoxy, ((C₁-C₆)alkyl)₂aminocarbonylamino(C₂-C₆)alkoxy, amino(C₂-C₆)alkoxycarbonylamino, (C₁-C₆)alkylamino(C₂-C₆)alkoxycarbonylamino, ((C₁-C₆)alkyl)₂amino(C₂-C₆)alkoxycarbonylamino, (C₂-C₉)heteroarylamino(C₂-C₆)alkoxy, barbituryl, (C₁-C₆)alkylcarbonylamino(C₁-C₆)alkylaminocarbonyl, amino(C₁-C₆)alkylcarbonylamino where the (C₁-C₆)alkyl is optionally substituted with one or two groups selected from but not limited to hydrogen, amino, hydroxyl, (C₁-C₆)alkoxy, carboxy, further substituted (C₂-C₉)heteroaryl, (C₆-C₁₀)aryl, (C₂-C₉)heterocycloalkyl, and cycloalkyl, or the two groups together make up a carbocycle; and R¹⁹carbonylamino where R¹⁹ is a nitrogen containing (C₂-C₉)heterocycloalkyl which is optionally substituted further with one or two groups selected from but not limited to (C₁-C,)alkyl, (C₂-C₆)alkoxy and hydroxy;

R⁹ is selected from the group consisting of hydrogen, (C₁-C₆)alkyl, (C₆-C₁₀)aryl, (C₆-C₁₀)aryl(C₁-C₆)alkyl, (C₁-C₆)alkylcarbonyl, (C₁-C₆)alkylcarbonyl(C₁-C₆)alkyl, (C₆-C₁₀)aryl(C₁-C₆)alkylcarbonyl, (C₆-C₁₀)aryl(C₁-C₆)alkylcarbonyl(C₁-C₆)alkyl, aminocarbonyl, (C₁-C₆)alkylaminocarbonyl, ((C₁-C₆)alkyl)₂aminocarbonyl and (C₁-C₆)alkoxycarbonyl;

R¹¹ and R¹² are each independently selected from the group consisting of hydrogen, (C₁-C₆)alkyl, (C₆-C₁₀)aryl, (C₆-C₁₀)aryl(C₁-C₆)alkyl, hydroxy, (C₁-C₆)alkoxy, hydroxy(C₁-C₆)alkyl, (C₁-C₆)alkoxy(C₁-C₆)alkyl, amino, (C₁-C₆)alkylamino, ((C₁-C₆)alkyl)₂amino, (C₁-C₆)alkylcarbonylamino, (C₃-C₈)cycloalkylcarbonylamino, (C₃-C₈)cycloalkyl(C₁-C₆)alkylcarbonylamino, (C₁-C₆)alkoxycarbonylamino, (C₁-C₆)alkylsulfonylamino, (C₆-C₁₀)arylcarbonylamino, (C₁-C₆)alkoxycarbonyl(C₁-C₆)alkylcarbonylamino, (C₆-C₁₀)aryl(C₁-C₆)alkylcarbonylamino, ((C₆-Cl o)aryl(C₁-C₆)alkylcarbonyl)((C₁-C₆)alkyl)amino, (C₁-C₆)alkylcarbonylamino(C₁-C₆)alkyl, (C₃-C₈)cycloalkylcarbonylamino(C₁-C₆)alkyl, (C₁-C₆)alkoxycarbonylamino(C₁-C₆)alkyl, (C₂-C₉)heterocycloalkylcarbonylamino(C₁-C₆)alkyl, (C₆-C₁₀)aryl (C₁-C₆)alkylcarbonylamino(C₁-C₆)alkyl, (C₂-C₉)heteroarylcarbonylamino(C₁-C₆)alkyl, (C₆-C₁₀)arylsulfonylamino, (C₁-C₆)alkylsulfonylamino(C₁-C₆)alkyl, aminocarbonylamino, (C₁-C₆)alkylaminocarbonylamino, halo(C₁-C₆)alkylaminocarbonylamino, ((C₁-C₆)alkyl)₂aminocarbonylamino, aminocarbonylamino(C₁-C₆)alkyl, (C₁-C₆)alkylaminocarbonylamino(C₁-C₆)alkyl, ((C₁-CE)alkyl)₂aminocarbonylamino(C₁-C₆)alkyl, halo(C₁-C₆)alkylaminocarbonylamino(C₁-C₆)alkyl, amino(C₁-C₆)alkyl, (C₁-C₆)alkylamino(C₁-C₆)alkyl, ((C₁-C₆)alkyl)₂amino(C₁-C₆)alkyl, carboxy(C₁-C₆)alkyl, (C₁-C₆)alkoxycarbonyl(C₁-C₆)alkyl, aminocarbonyl(C₁-C₆)alkyl and (C₁-C₆)alkylaminocarbonyl(C₁-C₆)alkyl.

Preferred compounds of formula I include those wherein R¹ is hydrogen, halo, cyano, nitro, trifluoromethyl, trifluoromethoxy, (C₁-C₆)alkyl, hydroxy or (C₁-C₆)alkylcarbonyloxy.

Other preferred compounds of formula I include those wherein c is 1; X is C(O); d is 1; and Z is oxygen.

Other preferred compounds of formula I include those wherein c is 1; X is C(O); d is 1; and Z is NR⁹ wherein R⁹ is hydrogen or (C₁-C₆)alkyl.

Other preferred compounds of formula I include those wherein c is 1; X is CH₂; d is 1; and Z is oxygen.

Other preferred compounds of formula I include those wherein c is 1; X is CH₂; d is 1; and Z is NR⁹ wherein R⁹ is hydrogen or (C₁-C₆) alkyl.

Other preferred compounds of formula I include those wherein c is 1; X is C(O); d is 1; and Z is CR¹¹R¹².

Other preferred compounds of formula I include those wherein c is 1; X is CH₂; d is 1; and Z is CR¹¹R¹²

Other preferred compounds of formula I include those wherein R⁴ is (R⁵)KC₆-C₁₀)aryl or (R⁵)KC₂-C₉)heteroaryl wherein f is 1 or 2.

Other preferred compounds of formula I include those wherein c is 1; X is C(O); d is 1; Z is oxygen, l and m are zero, k is 2, and W is CH.

Other preferred compounds of formula I include those wherein c is 1; X is C(O); d is 1; Z is oxygen, l and m are zero, k is 2, and W is nitrogen.

Other preferred compounds of formula I include those wherein c is 1; X is C(O); d is 1; Z is oxygen, l and m are zero, k is 3, and W is CH.

Other preferred compounds of formula I include those wherein c is 1; X is C(O); d is 1; Z is oxygen, l and m are zero, k is 3, and W is nitrogen.

Other preferred compounds of formula I include those wherein c is 1; X is C(O); d is 1; Z is NR⁹ wherein R⁹ is hydrogen or (C₁-C₆)alkyl, l and m are zero, k is 2, and W is CH.

Other preferred compounds of formula I include those wherein c is 1; X is C(O); d is 1; Z is NR⁹ wherein R⁹ is hydrogen or (C₁-C₆)alkyl, l and m are zero, k is 2, and W is nitrogen.

Other preferred compounds of formula I include those wherein c is 1; X is C(O); d is 1; Z is NR⁹ wherein R⁹ is hydrogen or (C₁-C₆)alkyl, l and m are zero, k is 3, and W is CH.

Other preferred compounds of formula I include those wherein c is 1; X is C(O); d is 1; Z is NR⁹ wherein R⁹ is hydrogen or (C₁-C₆)alkyl, l and m are zero, k is 3, and W is nitrogen.

Other preferred compounds of formula I include those wherein c is 1; X is C(O); d is 1; Z is oxygen, k and I are zero, m is 2, and W is CH.

Other preferred compounds of formula I include those wherein c is 1; X is C(O); d is 1; Z is oxygen, k and I are zero, m is 2, and W is nitrogen.

Other preferred compounds of formula I include those wherein c is 1; X is C(O); d is 1; Z is oxygen, k and I are zero, m is 3, and W is CH.

Other preferred compounds of formula I include those wherein c is 1; X is C(O); d is 1; Z is oxygen, k and I are zero, m is 3, and W is nitrogen.

Other preferred compounds of formula I include those wherein c is 1; X is C(O); d is 1; Z is NR⁹ wherein R⁹ is hydrogen or (C₁-C₆)alkyl, k and I are zero, m is 2, and W is CH.

Other preferred compounds of formula I include those wherein c is 1; X is C(O); d is 1; Z is NR⁹ wherein R⁹ is hydrogen or (C₁-C₆)alkyl, k and I are zero, m is 2, and W is nitrogen.

Other preferred compounds of formula I include those wherein c is 1; X is C(O); d is 1; Z is NR⁹ wherein R⁹ is hydrogen or (C₁-C₆)alkyl, k and I are zero, m is 3, and W is CH.

Other preferred compounds of formula I include those wherein c is 1; X is C(O); d is 1; Z is NR⁹ wherein R⁹ is hydrogen or (C₁-C₆)alkyl, k and I are zero, m is 3, and W is nitrogen.

Other preferred compounds of formula I include those wherein R⁴ is phenyl, q is 0 or 5 1, Q is (C₁-C₆)alkyl, and at least one R is selected from the following list of functional groups: (C₂-C₉)heteroarylaminocarbonyl, (C₂-C₉)heteroarylcarbonylamino, (C₁-C₆)alkylsulfonylaminocarbonyl, aminosulfonylaminocarbonyl, carboxy(C₁-C₆)alkylcyanoguanidino, carboxy, (C₂-C₉)heteroarylamino, (C₂-C₉)heteroarylsulfonyl, (C₂-C₉)heteroaryl (C₂-C₉)heteroaryloxy, (C₂-C₉)heteroarylcarbonyl, (C₂-C₉)heteroaryl(C₁-C₆)alkylcarbonyl, carboxy(C₁-C₆)alkylaminocarbonylamino, (C₂-C₉)heteroarylaminocarbonylamino, carboxy(C₁-C₆)alkylcarbonylamino, (C₂-C₉)heteroaryl(C₁-C₆)alkylamino, carboxy(C₁-C₆)alkylaminocarbonyl, carboxy(C₁-C₆)alkylsulfonylamino, (C₂-C₉)heteroarylaminosulfonyl, carboxy(C₁-C₆)alkylsulfonyl, carboxy(C₁-C₆)alkylamino, carboxy(C₁-C₆)alkylcarbonyl, carboxy(C₁-C₆)alkoxy, carboxy(C₁-C₆)alkoxycarbonylamino, hydroxyaminocarbonyl, (C₁-C₆)alkylsulfonylaminocarbonyl(C₁-C₆)alkoxy, (C₂-C₉)heteroaryl(C₁-C₆)alkoxy, carboxy(C₁-C₆)alkylamino(C₂-C₆)alkoxy, (C₂-C₉)heteroarylamino(C₂-C₆)alkoxy.

Other preferred compounds of formula I include those wherein R⁴ is phenyl, q is 0 or 1, Q is (C₁-C₆)alkyl, and at least one R⁵ is selected from the following list of functional groups: amino(C₁-C₆)alkylcarbonyl, (C₁-C₆)alkylamino(C₁-C₆)alkylcarbonyl, ((C₁-C₆)alkyl)₂amino(C₁-C₆)alkylcarbonyl, amino(C₁-C₆)alkylcarbonylamino, (C₁-C₆)alkylamino(C₁-C₆)alkylcarbonylamino, ((C₁-C₆)alkyl)₂amino(C₁-C₆)alkylcarbonylamino, amino(C₁-C₆)alkylureido, (C₁-C₆)alkylamino(C₁-C₆)alkylureido, ((C₁-C₆)alkyl)₂amino(C₁-C₆)alkylureido, amino(C₁-C₆)alkylsulfonylamino, (C₁-C₆)alkylamino(C₁-C₆)alkylsulfonylamino, ((C₁-C₆)alkyl)₂amino(C₁-C₆)alkylsulfonylamino, amino(C₁-C₆)alkylsulfonyl, (C₁-C₆)alkylamino(C₁-C₆)alkylsulfonyl, ((C₁-C₆)alkyl)₂amino(C₁-C₆)alkylsulfonyl, amino(C₁-C₆)alkylcyanoguanidino, (C₁-C₆)alkylamino(C₁-C₆)alkylcyanoguanidino, ((C₁-C₆)alkyl)₂amino(C₁-C₆)alkylcyanoguanidino, amino(C₁-C₆)alkylaminosulfonyl, (C₁-C₆)alkylamino(C₁-C₆)alkylaminosulfonyl, ((C₁-C₆)alkyl)₂amino(C₁-C₆)alkylaminosulfonyl, ((C₁-C₆)alkylamino)(C₆-C₁₀)aryl(C₁-C₆)alkyl, amino, amino(C₁-C₆)alkoxy, amino(C₁-C₆)alkoxycarbonylamino, (C₁-C₆)alkylamino, ((C₁-C₆)alkyl)₂amino, (C₆-C₁₀)arylamino, (C₆-C₁₀)aryl(C₁-C₆)alkylamino, amino(C₁-C₆)alkylamino, (C₂-C₉)heterocycloalkylamino, (C₂-C₉)heteroarylamino, (C₃-C₁₀)cycloalkyl(C₁-C₆)alkyl)amino, (amino(C₁-C₆)alkyl)aminocarbonyl, glycinamido, (C₁-C₆)alkylglycinamido, alaninamido, (C₁-C₆)alkylalaninamido, ((C₁-C₆)alkyl)2 amino(C₁-C₆)alkylcarbonylamino.

Other preferred compounds of formula I include those wherein R⁴ is phenyl, Q is (C₁-C₆)alkyl, q is 0 or 1, and at least one R⁵ is halo, (C₁-C₆)alkoxy, (C₁-C₆)alkyl, halo(C₁-C₆)alkyl.

Other preferred compounds of formula I include those wherein R⁴ is phenyl, q is 0 or 1, Q is (C₁-C₆)alkyl, and at least one R⁵ is selected from the following list of functional groups: aminocarbonyl(C₁-C₆)alkylureido, (C₁-C₆)alkylcarbonyl, (C₁-C₆)alkylsulfonylamino, (C₁-C₆)alkylsulfonylamino(C₁-C₆)alkylaminocarbonyl, aminosulfonyl, aminocarbonyl, ureido(C₁-C₆)alkylaminocarbonyl, aminocarbonyl(C₁-C₆)alkylaminocarbonyl, aminocarbonyl(C₁-C₆)alkylcarbonylamino, ureido(C₁-C₆)alkylcarbonylamino, (C₁-C₆)alkylcarbonylamino(C₁-Cr)alkylcarbonylamino, (C₁-C₆)alkylcarbonylamino(C₁-C₆)alkylaminocarbonylamino, ureido(C₁-C₆)alkylcarbonylamino, ureido, halo(C₁-C₆)alkylsulfonylamino, (C₁-C₆)alkylcarbonylamino(C₁-C₆)alkylaminocarbonyl.

Other preferred compounds of formula I include those wherein R⁴ is pyridyl, q is 0 or 1, Q is (C₁-C₆)alkyl, and at least one R⁵ is selected from the following list of functional groups: (C₂-C₉)heteroarylaminocarbonyl, (C₂-C₉)heteroarylcarbonylamino, (C₁-C₆)alkylsulfonylaminocarbonyl, aminosulfonylaminocarbonyl, carboxy(C₁-C₆)alkylcyanoguanidino, carboxy, (C₂-C₉)heteroarylamino, (C₂-C₉)heteroarylsulfonyl, (C₂-C₉)heteroaryl (C₂-C₉)heteroaryloxy, (C₂-C₉)heteroarylcarbonyl, (C₂-C₉)heteroaryl(C₁-C₆)alkylcarbonyl, carboxy(C₁-C₆)alkylaminocarbonylamino, (C₂-C₉)heteroarylaminocarbonylamino, carboxy(C₁-C₆)alkylcarbonylamino, (C₂-C₉)heteroaryl(C₁-C₆)alkylamino, carboxy(C₁-C₆)alkylaminocarbonyl, carboxy(C₁-C₆)alkylsulfonylamino, (C₂-C₉)heteroarylaminosulfonyl, carboxy(C₁-C₆)alkylsulfonyl, carboxy(C₁-C₆)alkylamino, carboxy(C₁-C₆)alkylcarbonyl, carboxy(C₁-C₆)alkoxy, carboxy(C₁-C₆)alkoxycarbonylamino, hydroxyaminocarbonyl, (C₁-C₆)alkylsulfonylaminocarbonyl(C₁-C₈)alkoxy, (C₂-C₉)heteroaryl(C₁-C₆)alkoxy, carboxy(C₁-C₆)alkylamino(C₂-C₆)alkoxy, (C₂-C₉)heteroarylamino(C₂-C₆)alkoxy.

Other preferred compounds of formula I include those wherein R⁴ is pyridyl, q is 0 or 1, Q is (C₁-C₆)alkyl, and at least one R is selected from the following list of functional groups: amino(C₁-C₆)alkylcarbonyl, (C₁-C₆)alkylamino(C₁-C₆)alkylcarbonyl, ((C₁-C₆)alkyl)₂amino(C₁-C₆)alkylcarbonyl, amino(C₁-C₆)alkylcarbonylamino, (C₁-C₆)alkylamino(C₁-CG)alkylcarbonylamino, ((C₁-C₆)alkyl)2amino(C₁-C₆)alkylcarbonylamino, amino(C₁-C₆)alkylureido, (C₁-C₆)alkylamino(C₁-C₆)alkylureido, ((C₁-C₆)alkyl)₂amino(C₁-C₆)alkylureido, amino(C₁-C₆)alkylsulfonylamino, (C₁-C₆)alkylamino(C₁-C₆)alkylsulfonylamino, ((C₁-C₆)alkyl)₂amino(C₁-C₆)alkylsulfonylamino, amino(C₁-C₆)alkylsulfonyl, (C₁-C₆)alkylamino(C₁-C₆)alkylsulfonyl, ((C₁-C₆)alkyl)₂amino(C₁-C₆)alkylsulfonyl, amino(C₁-C₆)alkylcyanoguanidino, (C₁-C₆)alkylamino(C₁-C₆)alkylcyanoguanidino, ((C₁-C₆)alkyl)₂amino(C₁-C₆)alkylcyanoguanidino, amino(C₁-C₆)alkylaminosulfonyl, (C₁-C₆)alkylamino(C₁-C₆)alkylaminosulfonyl, ((C₁-C₆)alkyl)₂amino(C₁-C₆)alkylaminosulfonyl, ((C₁-C₆)alkylamino)(C₆-C₁₀)aryl(C₁-C₆)alkyl, amino, amino(C₁-C₆)alkoxy, amino(C₁-C₆)alkoxycarbonylamino, (C₁-C₆)alkylamino, ((C₁-C₆)alkyl)₂amino, (C₆-C₁₀)arylamino, (C₆-C₁₀)aryl(C₁-C₆)alkylamino, amino(C₁-C₆)alkylamino, (C₂-C₉)heterocycloalkylamino, (C₂-C₉)heteroarylamino, (C₃-C₁₀)cycloalkyl(C₁-C₆)alkyl)amino, (amino(C₁-C₆)alkyl)aminocarbonyl, glycinamido, (C₁-C₆)alkylglycinamido, alaninamido, (C₁-C₆)alkylalaninamido, ((C₁-C₆)alkyl)₂ amino(C₁-C₆)alkylcarbonylamino.

Other preferred compounds of formula I include those wherein R⁴ is pyridyl, Q is (C₁-C₆)alkyl, q is 0 or 1, and at least one R⁵ is halo, (C₁-C₆)alkoxy, (C₁-C₆)alkyl, halo(C₁-C₆)alkyl.

Other preferred compounds of formula I include those wherein R⁴ is pyridyl, Q is (C₁-C₆)alkyl, q is 0 or 1, and at least one R⁵ is selected from: aminocarbonyl(C₁-C₆)alkylureido, (C₁-C₆)alkylcarbonyl, (C₁-C₆)alkylsulfonylamino, (C₁-C₆)alkylsulfonylamino(C₁-C₆)alkylaminocarbonyl, aminosulfonyl, aminocarbonyl, ureido(C₁-C₆)alkylaminocarbonyl, aminocarbonyl(C₁-C₆)alkyaminocarbonyl, aminocarbonyl (C₁-C₆)alkylcarbonylamino, ureido(C₁-C₆)alkylcarbonylamino, (C₁-C₆)alkylcarbonylamino(C₁-C₆)alkylcarbonylamino, (C₁-C₆)alkylcarbonylamino(C₁-C₆)alkylaminocarbonylamino, ureido(C₁-C₆)alkylcarbonylamino, ureido, halo(C₁-C₆)alkylsulfonylamino, (C₁-C₆)alkylcarbonylamino(C₁-C₆)alkylaminocarbonyl.

The present invention also relates to the pharmaceutically acceptable acid addition salts of compounds of the formula 1. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [1,1′-methylene-bis-(2-hydroxy-3- naphthoate)]salts.

The invention also relates to base addition salts of formula 1. The chemical bases that may be used as reagents to prepare pharmaceutically acceptable base salts of those compounds of formula I that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. Also included are pharmaceutically acceptable salts of basic compounds including hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate).

The compounds of this invention may contain olefin-like double bonds. When such bonds are present, the compounds of the invention exist as cis and trans configurations and as mixtures thereof.

Unless otherwise indicated, the alkyl, alkenyl and alkynyl groups referred to herein, as well as the alkyl moieties of other groups referred to herein (e.g., alkoxy), may be linear or branched, and they may also be cyclic (e.g., cyclopropyl, cyclobutyl, cyclopentyl, cyclohexyl or cycloheptyl) or be linear or branched and contain cyclic moieties. Unless otherwise indicated, halogen includes fluorine, chlorine, bromine, and iodine.

(C₃-C₁₀)Cycloalkyl when used herein refers to cycloalkyl groups containing zero to two levels of unsaturation such as cyclopropyl, cyclobutyl, cyclopentyl, cyclopentenyl, cyclohexyl, cyclohexenyl, 1,3-cyclohexadiene, cycloheptyl, cycloheptenyl, bicyclo[3.2.1]octane, norbornanyl etc.

(C₂-C₉)Heterocycloalkyl when used herein refers to, including but not limited to, pyrrolidinyl, tetrahydrofuranyl, dihydrofuranyl, tetrahydropyranyl, pyranyl, thiopyranyl, aziridinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, th iomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, chromanyl, etc, wherein the heterocycloalkyl group is optionally substituted by a group, including but not limited to, (C₁-C₆)alkyl, (C₁-C₆)alkoxy, halo, trifluoromethyl, trifluoromethoxy, or (C₁-C₆)alkylamino.

(C₂-C₉)Heteroaryl when used herein refers to, including but not limited to, furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-[1]pyrindinyl, benzo[b]thiophenyl, 5, 6, 7, 8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, quinolonyl, phthalazinyl, quinoxalinyl, quinazolinyl, benzoxazinyl; etc, wherein the heteroaryl group is optionally substituted by a group, including but not limited to, (C₁-C₆)alkyl, (C₁-C₆)alkoxy, halo, trifluoromethyl, trifluoromethoxy, or (C₁-C₅)alkylamino.

Aryl when used herein refers to phenyl or naphthyl.

The term “ureido”, as used herein, refers to an “amino-carbonyl-amino” moiety.

The term “acetyl”, as used herein, refers to an “alkyl-carbonyl” moiety wherein alkyl is defined as above.

The term “cyanoguanidino”, as used herein, refers to a functional group having the following formula:

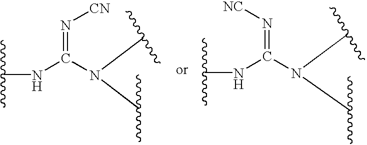

The term “(C₂-C₉)heterocycloalkyl(C═N—CN)amino”, as used herein refers to a functional group having the following formula:

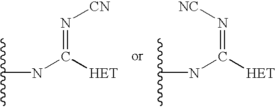

wherein “HET” refers to a (C₂-C₉)heterocyloalkyl or (C₂-C₉)heteroaryl group wherein the nitrogen of said group is the place of attachment.

The term “barbituryl”, as used herein refers to a functional group having the following formula

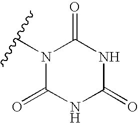

The term “mercapto”, as used herein, refers to a “HS-” moeity.

The term “alkoxy” refers to a radical of the formula ORₐ where Rₐ is an alkyl radical as define above, e.g., methoxy, ethoxyl.

The term “carboxy” refers to a radical of the formula —COOH.

The term “glycinamido” refers to a radical of the formula —NH—C(O)—CH₂—NH₂.

The term “cyano” refers to a radical of the formula —CN.

The term “nitro” refers to a radical of the formula —NO₂.

The term “nitroso” refers to a radical of the formula —NO.

The term “amidino” refers to a radical of the formula —C(NH)—NH₂.

The term “sulfonyl” refers to a radical of the formula —SO₂—.

The term “sulfinyl” refers to a radical of the formula —S(O)—.

The term “thio” refers to a radical of the formula —S—.

The term “oxo” refers to a radical of the formula ═O.

The term “formyl” refers to a radical of the formula —CHO.

The term “guanidino” refers to a radical of the formula —N(H)—C(NH)—NH₂.

The term “alaninamido” refers to a radical of the formula —NH—C(O)—CH(CH₃)—NH₂.

The compounds of this invention include all conformational isomers (e.g., cis and trans isomers) and all optical isomers of compounds of the formula I (e.g., enantiomers and diastereomers), as well as racemic, diastereomeric and other mixtures of such isomers. Compounds containing isotopic substitutions of atoms, such as deterium substitution of hydrogen, are also included in the scope of the present invention.

The present invention also relates to a pharmaceutical composition for treating or preventing autoimmune diseases (such as rheumatoid arthritis, type I diabetes (recent onset), lupus, inflammatory bowel disease, optic neuritis, psoriasis, multiple sclerosis, polymyalgia rheumatica, uveitis, and vasculitis), acute and chronic inflammatory conditions (such as osteoarthritis, adult Respiratory Distress Syndrome, Respiratory Distress Syndrome of infancy, ischemia reperfusion injury, glomerulonephritis, and chronic obstructive pulmonary disease (COPD)), allergic conditions (such as asthma and atopic dermatitis), inflammation associated with infection (such as viral inflammation (including influenza, hepatitis and Guillian-Barre), chronic bronchitis, tissue, cell, and chronic & acute solid organ transplant rejection (including xeno-transplantation), atherosclerosis, restenosis, HIV infectivity (co-receptor usage), and granulomatous diseases (including sarcoidosis, leprosy and tuberculosis) and sequelae associated with certain cancers such as multiple myeloma, comprising an amount of a compound of the formula I, a pharmaceutically acceptable salt or pro-drug thereof effective in treating or preventing such disorder or condition and a pharmaceutically acceptable carrier.

The compositions of the present invention may also limit the production of cytokines at inflammatory sites, including but not limited to TNF and IL-1, as a consequence of decreasing cell infiltration, providing benefit for diseases linked to TNF and IL-1 including congestive heart failure, pulmonary emphysema or dyspnea associated therewith, emphysema; HIV-1, HIV-2, HIV-3; cytomegalovirus (CMV), adenoviruses, Herpes viruses (Herpes zoster and Herpes simplex). They may also provide benefit for the sequelae associated with infection where such infection induces production of detrimental inflammatory cytokines such as TNF, e.g, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease, septic shock, cancer, trauma, and malaria, etc. The present invention also relates to a pharmaceutical composition for treating or preventing a disorder or condition that can be treated or prevented by inhibiting chemokine binding to the receptor CCR1 in a mammal, preferably a human, comprising an amount of a compound of the formula I, a pharmaceutically acceptable salt or pro-drug thereof, effective in treating or preventing such disorder or condition and a pharmaceutically acceptable carrier. Examples of such disorders and conditions are those enumerated in the preceding paragraph.

The present invention also relates to a method for treating or preventing a disorder or condition selected from autoimmune diseases (such as rheumatoid arthritis, type I diabetes (recent onset), lupus, inflammatory bowel disease, optic neuritis, psoriasis, multiple sclerosis, polymyalgia rheumatica, uveitis, and vasculitis), acute and chronic inflammatory conditions (such as osteoarthritis, adult Respiratory Distress Syndrome, Respiratory Distress Syndrome of infancy, ischemia reperfusion injury, glomerulonephritis, and chronic obstructive pulmonary disease (COPD)), allergic conditions (such as asthma and atopic dermatitis), inflammation associated with infection (such as viral inflammation (including influenza, hepatitis and Guillian-Barre), chronic bronchitis, tissue, cell, and chronic and acute solid organ transplant rejection (including xeno-transplantation), atherosclerosis, restenosis, HIV infectivity (co-receptor usage), and granulomatous diseases (including sarcoidosis, leprosy and tuberculosis) and sequelae associated with certain cancers such as multiple myeloma, comprising administering to a mammal in need of such treatment or prevention an amount of a compound of the formula I, or a pharmaceutically acceptable salt thereof, that is effective in treating or preventing such disorder or condition, including limiting the production of cytokines at inflammatory sites, including but not limited to TNF and IL-1, as a consequence of decreasing cell infiltration, thereof providing benefit for diseases linked to TNF and IL-1 including congestive heart failure, pulmonary emphysema or dyspnea associated therewith, emphysema; HIV-1, HIV-2, HIV-3; cytomegalovirus (CMV), adenoviruses, Herpes viruses (Herpes zoster and Herpes simplex). They may also provide benefit for the sequelae associated with infection where such infection induces production of detrimental inflammatory cytokines such as TNF e.g, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease, septic shock, cancer, trauma, and malaria, etc. The present invention also relates to a method for treating or preventing a disorder or condition that can be treated or prevented by antagonizing the CCR1 receptor in a mammal, preferably a human, comprising administering to a mammal in need of such treatment or prevention an amount of a compound of the formula I, a pharmaceutically acceptable salt or pro-drug thereof, that is effective in treating or preventing such disorder or condition.

The present invention also relates to a pharmaceutical composition for treating or preventing a disorder or condition selected from autoimmune diseases (such as rheumatoid arthritis, type I diabetes (recent onset), lupus, inflammatory bowel disease, optic neuritis, psoriasis, multiple sclerosis, polymyalgia rheumatica, uveitis, and vasculitis), acute and chronic inflammatory conditions (such as osteoarthritis, adult Respiratory Distress Syndrome, Respiratory Distress Syndrome of infancy, ischemia reperfusion injury, glomerulonephritis, and chronic obstructive pulmonary disease (COPD)), allergic conditions (such as asthma and atopic dermatitis), inflammation associated with infection (such as viral inflammation (including influenza, hepatitis and Guillian-Barre), chronic bronchitis, tissue, cell, and solid organ transplant rejection (including xeno-transplantation), atherosclerosis, restenosis, HIV infectivity (co-receptor usage), and granulomatous diseases (including sarcoidosis, leprosy and tuberculosis) and sequelae associated with certain cancers such as multiple myeloma, in a mammal, preferably a human, comprising a CCR1 receptor antagonizing effective amount of a compound of the formula I, or a pharmaceutically acceptable salt thereof, and a pharmaceutically acceptable carrier. Also included are using the pharmaceutical compositions to limit the production of cytokines at inflammatory sites, including but not limited to TNF and IL-1, as a consequence of decreasing cell infiltration, providing benefit for diseases linked to TNF and IL-1, including congestive heart failure, pulmonary emphysema or dyspnea associated therewith, emphysema; HIV-1, HIV-2, HIV-3; cytomegalovirus (CMV), adenoviruses, Herpes viruses (Herpes zoster and Herpes simplex). They may also provide benefit for the sequelae associated with infection where such infection induces production of detrimental inflammatory cytokines such as TNF e.g, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease; septic shock, cancer, trauma, and malaria, etc. The present invention also relates to a pharmaceutical composition for treating or preventing a disorder or condition that can be treated or prevented by antagonizing the CCR1 receptor in a mammal, preferably a human, comprising a CCR1 receptor antagonizing effective amount of a compound of the formula I, a pharmaceutically acceptable salt or pro-drug thereof, and a pharmaceutically acceptable carrier.

The present invention also relates to a method for treating or preventing a disorder or condition selected from autoimmune diseases (such as rheumatoid arthritis, type I diabetes (recent onset), lupus, inflammatory bowel disease, optic neuritis, psoriasis, multiple sclerosis, polymyalgia rheumatica, uveitis, and vasculitis), acute and chronic inflammatory conditions (such as osteoarthritis, adult Respiratory Distress Syndrome, Respiratory Distress Syndrome of infancy, ischemia reperfusion injury, glomerulonephritis, and chronic obstructive pulmonary disease (COPD)), allergic conditions (such as asthma and atopic dermatitis), inflammation associated with infection (such as viral inflammation (including influenza, hepatitis and Guillian-Barre), chronic bronchitis, tissue, cell, and solid organ transplant rejection (including xeno-transplantation) (chronic and acute), atherosclerosis, restenosis, HIV infectivity (co-receptor usage), and granulomatous diseases (including sarcoidosis, leprosy and tuberculosis) and sequelae associated with certain cancers such as multiple myeloma, The method of treatment of the present inventions also includes limiting the production of cytokines at inflammatory sites, including but not limited to TNF and IL-1, as a consequence of decreasing cell infiltration, providing benefit for diseases limited to TNF and IL-1 including congestive heart failure, pulmonary emphysema or dyspnea associated therewith, emphysema; HIV-1, HIV-2, HIV-3; cytomegalovirus (CMV), adenoviruses, Herpes viruses (Herpes zoster and Herpes simplex). They may also provide benefit for the sequelae associated with infection where such infection induces production of detrimental inflammatory cytokines such as TNF e.g, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease, septic shock, cancer, trauma, and malaria, etc., in a mammal, preferably a human, comprising administering to a mammal in need of such treatment or prevention a CCR1 receptor antagonizing effective amount of a compound of formula I, a pharmaceutically acceptable salt or pro-drug thereof.

### DETAILED DESCRIPTION OF THE INVENTION

The following reaction Schemes illustrate the preparation of the compounds of the present invention. Unless otherwise indicated a, c, d, f, k, l, m, W, X, Y, Z, R¹, and R⁴ in the reaction Schemes and the discussion that follow are defined as above.

R¹⁶ refers to an amino radical that can be unsubstituted, monosubstituted, disubstituted, cyclic or acyclic.

The reactions in Preparation D and Schemes 1, 2, 3, 4, 5, 6, and 7 are described in commonly assigned co-pending provisional application Ser. No. 60/193789, filed Mar. 31, 2000, the disclosure of which is incorporated herein by reference thereto.

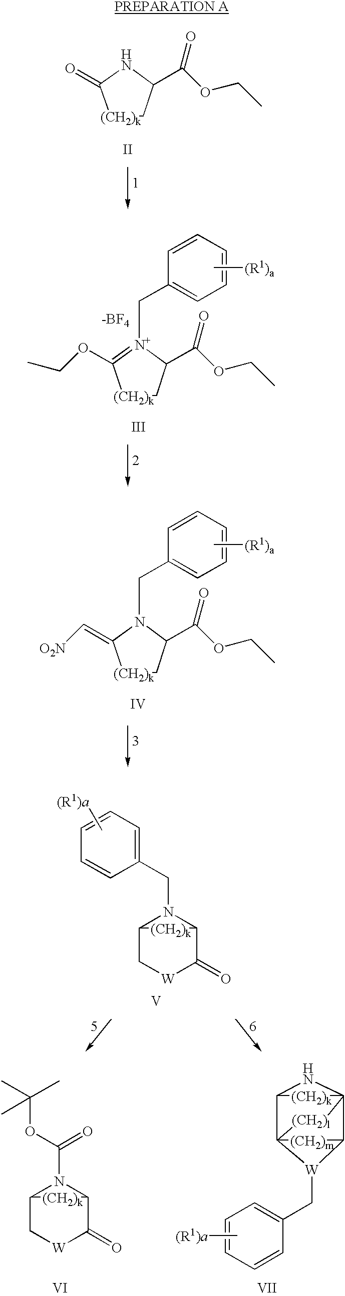

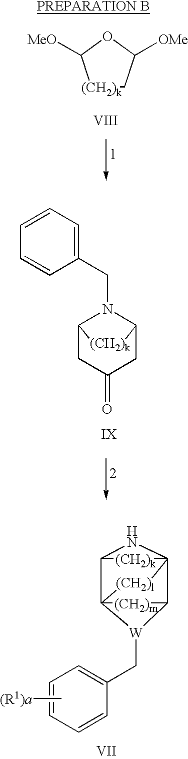

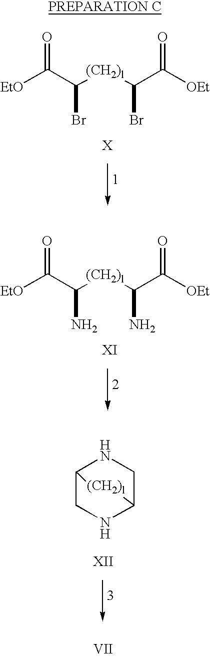

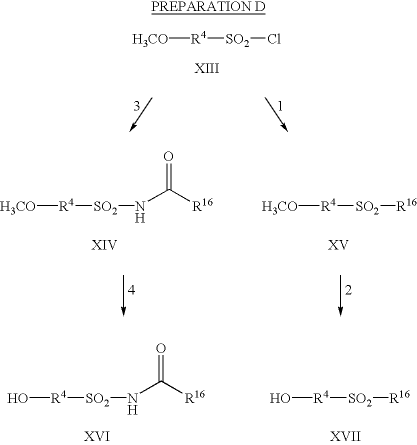

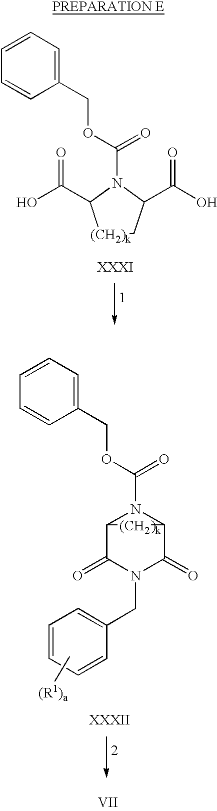

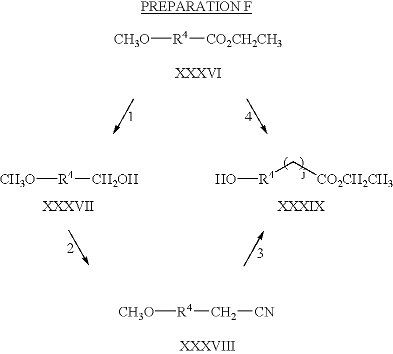

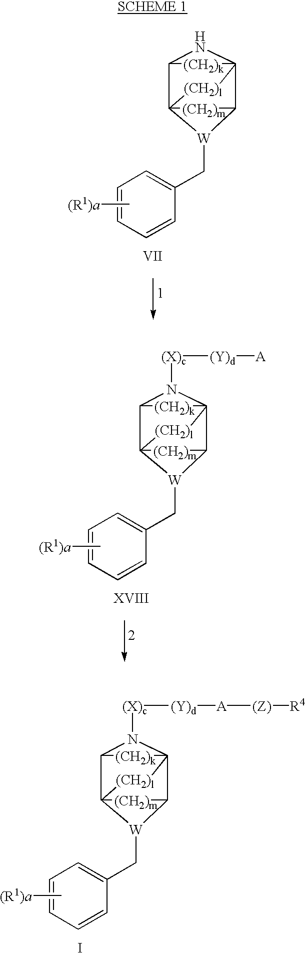

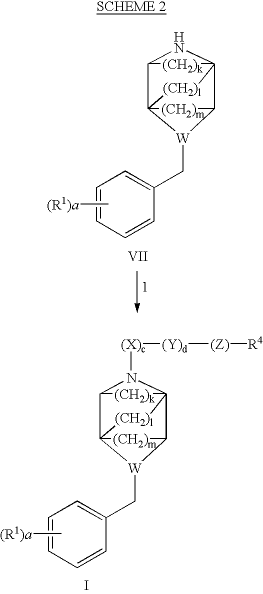

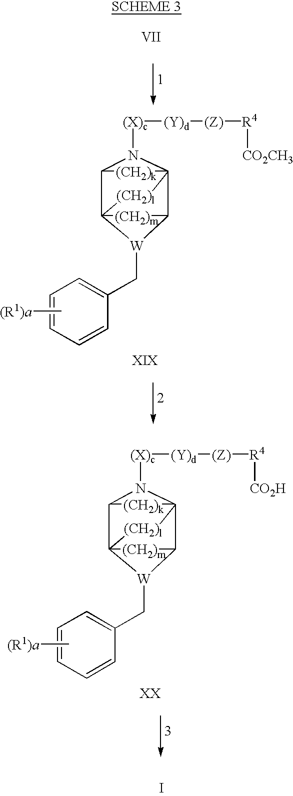

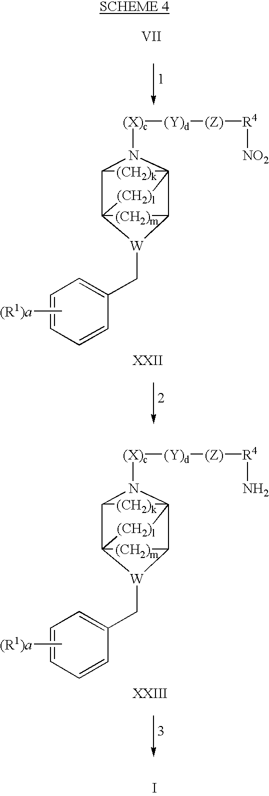

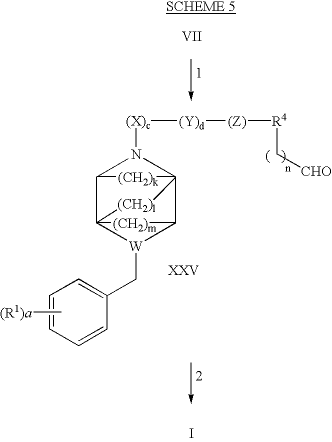

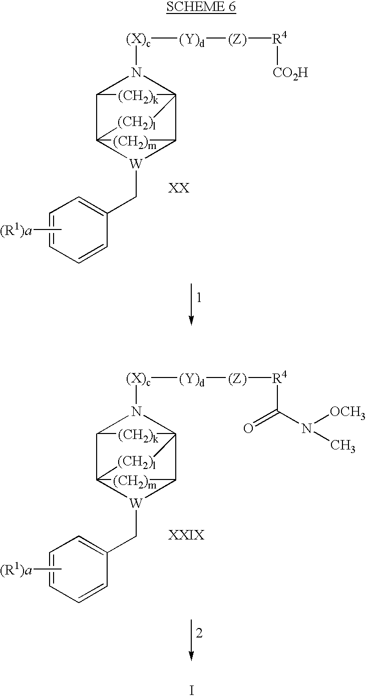

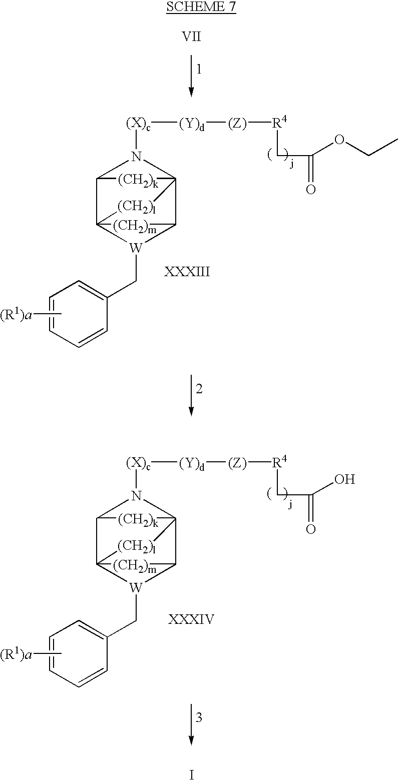

In reaction 1 of Preparation A, the compound of formula II, wherein k is 1, 2, 3, or 4, is treated with a base, such as sodium hydride, and an electrophile, such an optionally substituted benzyl bromide, in the presence of an aprotic solvent, such as tetrahydrofuran. The reaction mixture is stirred at ambient temperature for a time period between about 1 hour to about 12 hours, preferably about 10 hours. The resulting lactam is then converted to the corresponding compound of formula III by reacting with triethyloxonium tetrafluoroborate, in the presence of an aprotic solvent, such as dichloromethane. The reaction mixture is stirred at ambient temperature for a time period between about 1 hour to about 12 hours, preferably about 10 hours.

In reaction 2 of Preparation A the compound of formula III wherein k is 1, 2, 3, or 4, is converted to the corresponding compound of formula IV, by condensing III with nitromethane in the presence of a base, such as triethylamine, in the presence of an aprotic solvent, such as dichloromethane. The reaction mixture is stirred at ambient temperature for a time period between about 1 hour to about 16 hours, preferably about 10 hours.

In reaction 3 of Preparation A, the compound of formula IV wherein k is 1, 2, 3, or 4, is converted to the corresponding compound of formula V, wherein k is 1, 2, 3, or 4, and W is nitrogen, by first treating IV with a catalyst, such as palladium on carbon, in the presence of a protic solvent, such as methanol. The reaction mixture is shaken under a positive pressure of hydrogen gas for a time period between about 4 hours and about 16 hours, preferably about 12 hours. The resulting amino ester is then treated with a base, such as sodium methoxide, in an anhydrous protic solvent, such as methanol. The reaction mixture is stirred at ambient temperature for a time period between about 4 hours and about 16 hours, preferably about 10 hours.

In reaction 4 of Preparation A, the compound of formula V, wherein k is 1, 2, 3, or 4, and W is nitrogen, is converted to the corresponding compound of formula VII, wherein m is 1, 2, 3, or 4, k and I are 0, and W is nitrogen, by reducing V with a reducing agent, such as lithium aluminum hydride. The reaction is refluxed for a time period between about 2 hours and about 12 hours, preferable about 10 hours.

In reaction 5 of Preparation A, the compound of formula V, wherein k is 1, 2, 3, or 4, and W is nitrogen is converted to the corresponding compound of formula VI wherein k is 1, 2, 3, or 4, and W is nitrogen, by reacting V with an acylating agent, such as di-tert-butyl-dicarbonate, in the presence of a catalyst, such as 20% palladium hydroxide on carbon, and a protic solvent, such as methanol. The reaction is shaken under a positive pressure of hydrogen at a temperature between about ambient temperature and about 80° C., preferably about 60° C., for a time period between about 3 hours and about 13 hours, preferably about 10 hours.

In reaction 6 of Preparation A, the compound of formula VI, wherein k is 1, 2, 3, or 4, and W is nitrogen, is first reacted with an alkylating agent, such as an optionally substituted benzyl bromide, in the presence of a base, such as sodium hydride, and an aprotic solvent, such as tetrahydrofuran. The reaction is stirred for a time period between about 2 hours and about 12 hours, preferably about 10 hours. The resulting carbamate is then deprotected by treatment with an acid, such as trifluoroacetic acid, in the presence of an aprotic solvent, such as dichloromethane. The reaction is stirred at ambient temperature for a time period between about 1 hour and about 4 hours, preferably about 2 hours. The resulting amide is converted to the corresponding compound of formula VII, wherein k is 1, 2, 3, or 4, m and I are 0 and W is nitrogen, by reducing with a reducing agent, such as lithium aluminum hydride, in the presence of an aprotic solvent, such as tetrahydrofuran. The reaction is refluxed for a time period between about 2 hours and about 12 hours, preferable about 10 hours.

In reaction 1 of Preparation B, the compound of formula VII, wherein k is 1, 2, 3, or 4, is converted to the corresponding compound of formula IX by reacting with an amine, such as benzyl amine, and 3-oxo-pentanedioic acid in the presence of an acid such as 0.25 M aqueous hydrochloric acid. The reaction is stirred at ambient temperature for a period of time between about 30 minutes to about 2 hours, preferably 1.5 hours, and then heated to 50° C. for a period of time between about 1 hour and about 4 hours, preferably 2 hours.

In reaction 2 of Preparation B the compound of formula IX, where I is 1,2, 3, or 4, is converted to the corresponding compound of formula VII, wherein k is 1, 2, 3, or 4, l and m are 0 and W is CH, by first reacting with a phosphonium ylide of the formula

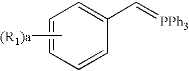

The reaction is refluxed for a period of time between about 4 hours and about 16 hours, preferably about 10 hours. The resulting olefin is then reduced by shaking under a positive pressure of hydrogen gas in the presence of a catalyst, such as 20% palladium hydroxide on carbon in the presence of a protic solvent, such as ethanol.

In reaction 1 Preparation C the compound of formula X wherein l is 1,2, 3, or 4, is converted to the corresponding compound of formula XI by first reacting with sodium azide in the presence of a protic solvent such as ethanol. The reaction is refluxed for a period of time between about 3 hours and about 12 hours, preferably about 10 hours. The resulting diazide is then reduced in the presence of platinum oxide and a polar solvent such as ethanol. The reaction is shaken under a positive pressure of hydrogen for a period of time between about 3 hours and about 12 hours, preferably about 10 hours.

In reaction 2 of Preparation C the compound of formula XI, wherein l is 1, 2, 3, or 4, is converted to the corresponding compound of formula XII by first treating compound XI with a base, such as sodium methoxide, in the presence of a protic solvent such as methanol. The reaction is refluxed for a period of time between about 3 hours and about 12 hours, preferably about 10 hours. The resulting piperazine-dione is converted to the corresponding compound of the formula XII by treating with a reducing agent, such as lithium aluminum hydride, in an aprotic solvent, such as tetrahydrofuran. The reaction is refluxed for a period of time between about 3 hours and about 12 hours, preferably about 10 hours.

In reaction 3 of Preparation C the compound of the formula XII, wherein l is 1,2, 3, or 4, is converted to the corresponding compound of formula VII, wherein I is 1, 2, 3, or 4, k and m are 0, and W is nitrogen, by reacting with an optionally substituted benzaldehyde compound of the formula

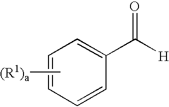

in the presence of a base, such as triethylamine, and a reducing agent, such as sodium triacetoxyborohydride, in an aprotic solvent, such as 1,2-dichloroethane. The reaction mixture is stirred at ambient temperature for a time period between about 1 hour to about 12 hours, preferably about 10 hours.

In reaction 1 of the Preparation D, the compound of formula XII is converted to the corresponding compound of formula XV by reacting XII with an appropriate amine of the formula, HR¹⁶ in the presence of a polar aprotic solvent, such as methylene chloride. The reaction mixture is stirred, at ambient temperature, for a time period between about 1 hour to about 24 hours, preferably about 12 hours.

In reaction 2 of Preparation D, the compound of formula XV is converted to the corresponding compound of formula XVII by reacting XV with thiophenol in the presence of a base, such as sodium hydride, and a polar aprotic solvent, such as dimethylformamide. The reaction is heated to reflux for a time period between about 1 hour to about 10 hours, preferably about 4 hours.

In reaction 3 of Preparation D, the compound of formula XII is converted to the corresponding compound of formula XIV by reacting XII with sodium cyanate in the presence of pyridine and a polar aprotic solvent, such as acetonitrile. The reaction is stirred, at ambient temperature, for a time period between about 2 hours to about 18 hours, preferably about 10 hours. An appropriate amine of the formula HR¹⁶, is then added and the reaction mixture so formed is stirred, at ambient temperature, for a time period between about 2 hours to about 24 hours, preferably about 8 hours.

In reaction 4 of Preparation D, the compound of formula XIV is converted to the corresponding compound of formula XVI according to the procedure described above in reaction 2 of Preparation D.

In reaction 1 of Preparation E the compound of formula XXXI wherein k is 1, 2, 3, or 4, is treated with an anhydride, such as acetic anhydride. The reaction mixture is heated to 70° C. for a time period between 8 and 15 hours, preferably about 12 hours. The resulting mixture is then concentrated and the anhydride is treated with an optionally substituted benzyl amine in the presence of an aprotic solvent such as toluene. The reaction mixture is stirred at ambient temperature for a time period between 1 and 16 hours, preferably about 10 hours, and then treated with an anhydride, such as acetic anhydride, and heated to reflux for a time period between 1 and 20 hours, preferably about 16 hours.

In reaction 2 of Preparation E the compound of formula XXXII, wherein k is 1, 2, 3, or 4, is converted to the corresponding compound of formula VII, wherein k is 1, 2, 3, or 4, 1 and m are 0, and W is nitrogen, by first treating XXXII with a catalyst, such as palladium on carbon, in the presence of a hydrogen source, such as cyclohexadiene, and a protic solvent, such as ethanol. The reaction mixture is stirred at ambient temperature for a time period between 1 hour and 4 hours, preferably about 1.5 hours. The resulting compound is then treated with a reducing agent, such as Red-Al in an aprotic solvent such as toluene. The reaction is heated to 60° C. for time period between 2 hours and 6 hours, preferably about 4 hours.

In reaction 1 of Preparation F the compound of formula XXXVI is converted to the corresponding compound of the formula XXXVII by treating with a reducing agent, such as lithium aluminum hydride, in an aprotic solvent, such as tetrahydrofuran. The reaction mixture is heated to reflux for a time period between 1 hour and 6 hours, preferably about 2 hours.

In reaction 2 of Preparation F the compound of formula XXXVII is converted to the corresponding compound of the formula XXXVIII by first treating with an activating agent such as sulfonyl chloride, in the presence of an aprotic solvent, such as chloroform. The reaction is heated to reflux, for a time period between about 1 hour to about 10 hours, preferably about 3 hours. The resulting alkyl chloride is then treated with a cyanide source, such as potassium cyanide, in the presence of an aprotic solvent, such as acetonitrile. The reaction mixture is stirred at ambient temperature for a time period between about 1 hour to about 10 hours, preferably about 3 hours.

In reaction 3 of Preparation F the compound of formula XXXVIII is converted to the compound of formula XXXIX, wherein j is 1, by first treating the cyanide with base, such as potassium hydroxide in water. The reaction mixture is heated to reflux for a time period between about 1 hour to about 10 hours, preferably about 6 hours. The resulting methyl ether is deprotected by treatment with acid, such as 47% aqueous hydrogen bromide. The reaction mixture is heated to reflux for a time period between about 10 hours to about 30 hours, preferably about 24 hours. The deprotected phenol acid is finally converted to the corresponding compound of formula XXXIX, wherein j is 1, by refluxing in ethanol in the presence of an acid, such as sulfuric acid, for a time period between about 8 hours to about 16 hours, preferably about 12 hours.

In reaction 4 of Preparation F the compound of formula XXXVI is converted to the corresponding compound of formula XXXIX, where in j is 2 or 3, by first treating the ester with a reducing agent, such as diisobutylaluminum hydride, in the presence of a aprotic solvent, such as toluene. The resulting aldehyde is treated with a phosphonium ylide derived from the phosphonium salt of the formula

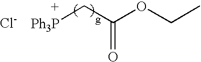

wherein g is 1 or 2, in the presence of an aprotic solvent, such as tetrahydrofuran. The reaction is refluxed for a time period between about 4 hours to about 16 hours, preferably about 10 hours. The resulting olefin is then reduced by shaking under a positive pressure of hydrogen in the presence of a catalyst, such as 20% palladium hydroxide on carbon, in the presence of a protic solvent such as ethanol. The methyl ether is deprotected according to the procedure described for reaction 2 of Scheme D.

In reaction 1 of Scheme 1, the compound of formula VII is converted to the corresponding compound of formula XVIII by reacting VII with a compound of the formula, A-(X)_{c}-(Y)_{d}-A, wherein A is chloro or bromo, in the presence of a base, such as triethylamine, and a polar aprotic solvent, such as methylene chloride. The reaction is stirred at a temperature between about −10° C. to about 10° C., for a time period between about 15 minutes to about 90 minutes, preferably about 30 minutes.

In reaction 2 of Scheme 1, the compound of formula XVIII is converted to the corresponding compound of formula I by reacting XVIII with a compound of the formula, H-(Z)-R⁴ wherein d Z is oxygen, which is either commercially available or is prepared as in Preparations D and F, in the presence of potassium carbonate, potassium iodide and an aprotic solvent, such as butanone. The reaction is heated to reflux for a time period between about 4 hours to about 8 hours, preferably about 6 hours.

In reaction 1 of Scheme 2, the compound of formula VII is converted to the corresponding compound of formula I by reacting VII with a compound of the formula, A-(X)_{c}-(Y)_{d}-(Z)-R⁴, wherein A is chloro or bromo, in the presence of a base, such as triethylamine, and a polar aprotic solvent, such as methylene chloride. The reaction is stirred at a temperature between about −10° C. to about 10° C., for a time period between about 15 minutes to about 90 minutes, preferably about 30 minutes.

In reaction 1 of Scheme 3, the compound of formula VII is converted to the corresponding compound of formula XIX according to the procedure described above in reaction 2 of Scheme 1.

In reaction 2 of Scheme 3, the compound of formula XIX is converted to the corresponding compound of formula XX by reacting XIX with lithium hydroxide monohydrate in the presence of methanol, tetrahydrofuran and water. The reaction mixture is stirred overnight at ambient temperature.

In reaction 3 of Scheme 3, the compound of formula XX is converted to the corresponding amide or acylsulfonamide of formula I, by reacting XX with an appropriate amine or sulfonamide in the presence of 4-dimethylaminopyridine, 1-(3-dimethylaminopropyl)-3-ethylcarbodiimine and a polar aprotic solvent, such as methylene chloride. The resulting reaction mixture is stirred overnight at ambient temperature.

In reaction 1 of Scheme 4, the compound of formula VII is converted to the corresponding compound of formula XXII according to the procedure described above in reaction 2 of Scheme 1.

In reaction 2 of Scheme 4, the compound of formula XXII is converted to the corresponding compound of formula XXIII by hydrogenating XXII in the presence of a catalyst, such as platinum on carbon, and a polar protic solvent, such as ethanol. The reaction is carried out under a pressure between about 30 psi to about 40 psi, preferably about 35 psi, for a time period between about 15 minutes to about 1 hour, preferably 30 minutes.

In reaction 3 of Scheme 4, for urea formation, the compound of formula XXIII is converted to the corresponding urea of formula I, by first reacting XXIII with 4-nitrophenyl chloroformate in the presence of a base, such as pyridine, and a polar aprotic solvent, such as methlyene chloride, followed by reacting the intermediate so formed with an appropriate amine. The reaction mixture, so formed, is allowed to stir overnight at ambient temperature. The compound of formula XXIII is reacted with an appropriate sulfonyl chloride to form the sulfonamides of formula I, in the presence of a base, such as triethylamine, and a polar aprotic solvent, such as methylene chloride. The reaction is stirred overnight at ambient temperature. For cyanoguanidine formation, the compound of formula I is first treated with sodium hydride in an aprotic solvent, such as tetrahydrofuran, followed by reacting, the intermediate so formed with dimethyl-N-cyanodithio iminocarbonate. The resulting reaction mixture is heated to reflux overnight. The N-cyano-S-methyl-isothiourea intermediate is then reacted with an appropriate amine in the presence of a polar protic solvent, such as methanol, to form the cyanoguanidine of formula I . For amide formation, the compound of formula XXIII is reacted with an acid, such as 3-tert-butoxycarbonylaminopropionic acid in the presence of N-methylmorpholine, O-benzotriazole-1-yl-N,N,N, N -tetramethyluronium hexafluorophosphate and a polar aprotic solvent, such as methylene chloride, to form the amide of formula I. For secondary amine formation the compound of formula XXIIII is reacted with an appropriate aldehyde to form the amine of formula I according to the procedure described above in reaction 1 of Preparation B.

In reaction 1 of Scheme 5, the compound of formula VII is converted to the corresponding compound of formula XXV, wherein n is 0, 1, 2, 3 or 4, according to the procedure described above in reaction 2 of Scheme 1.

In reaction 2 of Scheme 5, the compound of formula XXV is converted to the corresponding amine of formula I by reacting XXV with an appropriate amine in the presence of a 10:1 ratio solution of dichloroethane/acetic acid. The reaction mixture is stirred, at ambient temperature, for a time period between about 30 minutes to about 2 hours, preferably about 1 hour. A reducing agent, such as sodium cyanoborohydride is than added to the mixture and the reaction is allowed to stir overnight at ambient temperature. If the amine thus formed is secondary, the compound of formula I may further be reacted according to the procedure described above in reaction 3 of Scheme 4, to provide ureas, sulfonamides, cyanoguanidinos, or amides.

In reaction 1 of Scheme 6, the acid compound of formula XX is converted to the corresponding compound of formula XXIX by treating XX with thionyl chloride neat or in an aprotic solvent, at ambient temperature, for a time period between about 1 hour to about 24 hours, preferably 1 hour. The acid chloride so formed is dissolved in a polar aprotic solvent with a compound of the formula, (H₃CO)(H₃C)NH·HCl, in the presence of an amine base, such as triethylamine. The reaction mixture is stirred, at ambient temperature, for a time period between about 1 hour to about 48 hours, preferably about 12 hours.

In reaction 2 of Scheme 6, the amide compound of formula XXIX is converted to the corresponding compound of formula I by reacting XXIX with a (C₂-C₉)heteroaryl lithium reagent in the presence of a polar aprotic solvent at a temperature between about −100° C. to ambient temperature, preferably about −78° C. The resulting reaction mixture is stirred for a time period between about 1 hour to about 24 hours, preferably about 12 hours, at a temperature between about −78° C. to about 50° C., preferably about 20° C.

In reaction I of Scheme 7, the compound of formula VII is converted to the corresponding compound of formula XXXIII, wherein j is 1, 2, or 3, according to the procedure described above in reaction 2 of Scheme 1.

In reaction 2 of Scheme 7, the compound of formula XXXIII, wherein j is 1, 2, or 3, is converted to the corresponding compound of formula XXXIV, wherein j is 1, 2, or 3, according to the procedure described above in reaction 2 of Scheme 3.

In reaction 3 of Scheme 7 the compound of formula XXXIV, wherein j is 1, 2, or 3, is converted to the corresponding amide or acylsulfonamide of the formula I, wherein j is 1, 2, or 3, by treating with an appropriate amine or sulfonamide according to the procedure described above in reaction 3 of Scheme 3. The compound of formula XXXIV, wherein j is 1, 2, or 3, is converted to the corresponding compound of formula I according to the procedures described above for Scheme 6.

Unless indicated otherwise, the pressure of each of the above reactions is not critical. Generally, the reactions will be conducted at a pressure of about one to about three atmospheres, preferably at ambient pressure (about one atmosphere).

The compounds of the formula I which are basic in nature are capable of forming a wide variety of different salts with various inorganic and organic acids. Although such salts must be pharmaceutically acceptable for administration to animals, it is often desirable in practice to initially isolate a compound of the formula I from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, a solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [1,1′-methylene-bis-(2-hydroxy-3-naphthoate)] salts.

Those compounds of the formula I which are also acidic in nature, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the herein described acidic compounds of formula I. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

Compounds of the formula I and their pharmaceutically acceptable salts (hereinafter also referred to, collectively, as “the active compounds”) are potent antagonists of the CCR1 receptor. The active compounds are useful in the treatment or prevention of autoimmune diseases (such as rheumatoid arthritis, type I diabetes (recent onset), lupus, inflammatory bowel disease, optic neuritis, psoriasis, multiple sclerosis, polymyalgia rheumatica, uveitis, and vasculitis), acute and chronic inflammatory conditions (such as osteoarthritis, adult Respiratory Distress Syndrome, Respiratory Distress Syndrome of infancy, ischemia reperfusion injury, glomerulonephritis, and chronic obstructive pulmonary disease (COPD)), allergic conditions (such as asthma and atopic dermatitis), inflammation associated with infection (such as viral inflammation (including influenza, hepatitis and Guillian-Barre), chronic bronchitis, chronic and acute tissue, cell, and solid organ transplant rejection (including xeno-transplantation), atherosclerosis, restenosis, HIV infectivity (co-receptor usage), and granulomatous diseases (including sarcoidosis, leprosy and tuberculosis) and sequelae associated with certain cancers such as multiple myeloma. Compounds in this series may also limit the production of cytokines at inflammatory sites, including but not limited to TNF and IL-1, as a consequence of decreasing cell infiltration, providing benefit for diseases linked to TNF and IL-1 including congestive heart failure, pulmonary emphysema or dyspnea associated therewith, emphysema; HIV-1, HIV-2, HIV-3; cytomegalovirus (CMV), adenoviruses, Herpes viruses (Herpes zoster and Herpes simplex). They may also provide benefit for the sequelae associated with infection where such infection induces production of detrimental inflammatory cytokines such as TNF e.g, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease, septic shock, cancer, trauma, and malaria, etc.

The activity of the compounds of the invention can be assessed according to procedures know to those of ordinary skill in the art. Examples of recognized methods for determining CCR1 induced migration can be found in Coligan, J. E., Kruisbeek, A. M., Margulies, D. H., Shevach, E. M., Strober, W. editors: Current Protocols In Immunology, 6.12.1-6.12.3. (John Wiley and Sons, NY, 1991). One specific example of how to determine the activity of a compound for inhibiting migration is described in detail below.

### Chemotaxis Assay

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-1α (Peprotech, Inc., P.O. Box 275, Rocky Hill N.J.) or other test agonists, are placed into the lower chambers of the Boyden chamber. A polycarbonate filter is then applied and the upper chamber fastened. The amount of agonist chosen is that determined to give the maximal amount of chemotaxis in this system (e.g., 1 nM for MIP-1α should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber.

After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotant is the % inhibition for the compound which can then be plotted using standard graphics techniques against the concentration of compound used. The 50% inhibition point is then determined using a line fit analysis for all concentrations tested. The line fit for all data points must have an coefficient of correlation (R squared) of >90% to be considered a valid assay.

All of the compounds of the invention illustrated in the following examples had IC₅₀ of less than 10 μM, in the Chemotaxis assay.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation. The active compounds of the invention may also be formulated for sustained delivery.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents can be formulated for sustained delivery according to methods well known to those of ordinary skill in the art. Examples of such formulations can be found in U.S. Pat. Nos. 3,538,214, 4,060,598, 4,173,626, 3,119,742, and 3,492,397.

The compounds of the invention can also be utilized in combination therapy with immunosuppressant agents including but not limited to rapamycin, cyclosporin A, FK-506, Cellcept®; azathioprine, and IL-2R inhibitory antibodies or with classical anti-inflammatory agents (e.g. cyclooxygenase/lipoxygenase inhibitors) such as but not limited to, tenidap, aspirin, acetaminophen, naproxen and piroxicam or with cytokine inhibitory agents including but not limited to ENBREL.

The following Examples illustrate, but are not limited to, the preparation of the compounds of the present invention.

### EXAMPLE 1

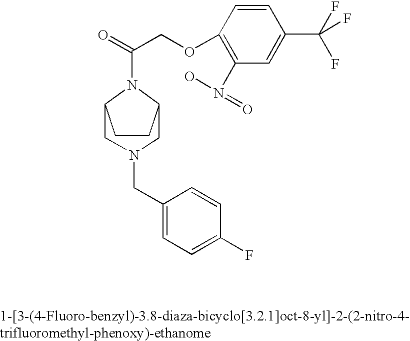

### 1-(4-Fluoro-benzyl )-5-oxo-pyrrolidine-2-carboxylic Acid Ethyl Ester

To a 0° C. solution of 5-oxo-pyrrolidine-2-carboxylic acid ethyl ester (15 g, 95.0 mmol) and 4-fluorobenzylbromide (19.7 g, 104.0 mmol) in tetrahydrofuran (800 ml) is added sodium hydride (60% dispersion, 5.7 g, 142.0 mmol) in four portions. The reaction mixture is held at 0° C. for thirty minutes and then warmed to ambient temperature for 3 hours. The resulting mixture is diluted with diethyl ether and extracted with saturated aqueous ammonium chloride. The combined organics are dried over magnesium sulfate, filtered and concentrated in vacuo. Silica gel chromatography gave the title compound (18.11 g, 72%)

### 1-(4-Fluoro-benzyl )-5-nitromethylene-pyrrolidine-2-carboxylicacid Ethyl Ester

To a solution of triethyloxonium tetrafluoroborate (5.52 g, 29.0 mmol) and molecular seives (3 angstroms, 35 g) in dry dichloromethane (30 ml) is added 1-(4-fluoro-benzyl)-5-oxo-pyrrolidine-2-carboxylic acid ethyl ester (7.0 g, 26.4 mmol) dropwise over fifteen minutes. The reaction mixture is stirred at ambient temperature overnight, then filtered through a glass frit and washed with dichloromethane. The collected precipitate is dried in vacuo overnight in the presence of phophorous pentoxide to give the iminium tetrafluoroborate (9.10 g, 95%).

To a solution of the iminium tetrafluoroborate (9.10 g, 25.1 mmol) in dry dichloromethane (35 ml) is added triethylamine (3.8 ml, 27.6 mmol). The reaction mixture is stirred at ambient temperature for ten minutes, and then nitromethane (6.8 ml, 125.0 mmol) is added. The resulting mixture is stirred at ambient temperature overnight, then concentrated in vacuo. The residue is diluted with chloroform, washed with 10% aqueous hydrochloric acid, water, and brine. The organics are dried over magnesium sulfate, filtered, and concentrated in vacuo. Silica gel chromatography gave the title compound (4.19 g, 54%) in addition to recoverd 1-(4-fluoro-benzyl)-5-oxo-pyrrolidine-2-carboxylic acid ethyl ester (7.92 g, 32%).

### 5-Aminomethyl-1-(4-fluoro-benzyl)-pyrrolidine-2-carboxylicacid ethyl ester

To a solution of 1-(4-fluoro-benzyl)-5-nitromethylene-pyrrolidine-2-carboxylic acid ethyl ester (5.24 g, 17.0 mmol) in methanol (50 ml) in a par bottle is added palladium on carbon (10%, 2.5 g). The resulting suspension is subjected to hydrogen gas (40 psi) overnight, filtered through a pad of celite, the filter cake is washed with methanol. The filtrate is concentrated in vacuo to give a mixture of the title compound and 8-(4-fluoro-benzyl)-3,8-diaza-bicyclo[3.2.1]octan-2-one (3.56 g).

### 8-(4-Fluoro-benzyl)-3,8-diaza-bicyclo[3.2.1]octan-2-one

To a solution of a mixture of 5-aminomethyl-1-(4-fluoro-benzyl)-pyrrolidine-2-carboxylic acid ethyl ester (2.19 g, 7.82 mmol) in dry methanol (40 ml) is added sodium methoxide (0.84 g, 15.6 mmol). The resulting mixture is stirred at ambient temperature overnight, diluted with ethyl acetate, and washed with saturated aqueous sodium hydrogen carbonate. The combined organics are dried over magnesium sulfate, filtered and concentrated in vacuo to give the title compound (1.34 g, 73%).

### 2-Oxo-3,8-diaza-bicyclo[3.2.1]octane-8-carboxylic acid tert-butyl ester

To a solution 8-(4-fluoro-benzyl)-3,8-diaza-bicyclo[3.2.1]octan-2-one (1.50 g, 6.40 mmol) and di-tert-butyl-dicarbonate (1.67 g, 7.6 mmol) in ethanol (50 ml) in a par bottle is added palladium hydroxide on carbon (20%, 1.0 g). The resulting suspension is subjected to hydrogen gas (40 psi) at 60° C. overnight, filtered through a pad of celite, the filter cake is washed with ethyl acetate. The filtrate is concentrated in vacuo to give the title compound (1.22 g, 84%).

### 3-(4-Fluoro-benzyl )-2-oxo-3.8-diaza-bicyclo[3.2.1]octane-8-carboxylicacid tert-but l ester

To a 0° C. solution of 2-oxo-3,8-diaza-bicyclo[3.2.1]octane-8-carboxylic acid tert-butyl ester (0.35 g, 1.54 mmol) in tetrahydrofuran (8.0 ml) is added 4-fluorobenzylbromide (0.21 ml, 1.70 mmol) followed by sodium hydride (60% dispersion, 0.092 g, 2.3 mmol). The resulting mixture is stirred at 0° C. for 30 minutes and then warmed to ambient temperature overnight. The reaction mixture is diluted with diethyl ether and washed with saturated aqueous ammonium chloride. The combined organics are dried over magnesium sulfate, filtered and concentrated in vacuo to give the title compound, which is taken on crude.

### 3-(4-Fluoro-benzyl)-3,8-diaza-bicyclo[3.2.1]octan-2-one

To a solution of 3-(4-fluoro-benzyl)-2-oxo-3,8-diaza-bicyclo[3.2.1]octane-8-carboxylic acid tert-butyl ester in dichloromethane (15.0 ml) is added trifluoroacetic acid (3.0 ml). The resulting mixture is stirred at ambient temperature for three hours, then basified with 1N aqueous sodium hydroxide and extracted with dichloromethane. The combined organics are dried over magnesium sulfate, filtered and concentrated in vacuo to give the title compound (0.297 g, 82% over 2 steps).

### 3-(4-Fluoro-benzyl)-3,8-diaza-bicyclo[3.2.1]octane

To a 0° C. solution of 3-(4-fluoro-benzyl)-3,8-diaza-bicyclo[3.2.1]octan-2-one (0.284 g, 1.21 mmol) in tetrahydrofuran (6.0 ml) is added lithium aluminum hydride (1.0 M in tetrahydrofuran, 6.1 ml, 6.1 mmol) dropwise. The reaction mixture is slowly warmed to ambient temperature and then refluxed over night. The resulting mixture is cooled to 0° C. and slowly quenched with water and IN aqueous sodium hydroxide, then filtered through a pad of celite, the filter cake is washed with ethyl acetate. The filtrate is concentrated in vacuo to give the title compound (0.25 g, 94%).

### 2-chloro-1-[3-(4-fluoro-benzyl)-3,8-diaza-bicyclo[3.2.1]oct-8-yl]-ethanone

To a solution of 3-(4-fluoro-benzyl)-3,8-diaza-bicyclo[3.2.1]octane (0.71g, 3.22 mmol) in dry dichloromethane (30 ml) at 0° C. is added triethylamine (0.45 ml, 3.22 mmol) followed by choroacetyl chloride (0.27 ml, 3.54 mmol). The resulting reaction mixture is stirred for two hours and concentrated in vacuo. Silica gel chromatography gave the title compound (0.66 g, 69%).

### 1-[3-(4-Fluoro-benzyl)-3,8-diaza-bicyclo[3.2.1]oct-8-yl]-2-(2-nitro-4-trifluoromethyl-phenoxy)-ethanone

To a solution of 2-chloro-1-[3-(4-fluoro-benzyl)-3,8-diaza-bicyclo[3.2.1]oct-8-yl]-ethanone (0.119, 0.37 mmol) in butanone (4 ml) is added 2-nitro-4-trifluoromethyl-phenol (0.300 g, 0.41 mmol), potassium carbonate (0.15 g, 1.09 mmol) and potassium iodide (0.18 g, 1.09 mmol). The resulting mixture is stirred at reflux for 7 hours. The reaction is then cooled, diluted with ethyl acetate and washed with brine. The organic layers are dried over magnesium sulfate, filtered and concentrated in vacuo. Silica gel chromatography gave the title compound (0.10 g, 58%).

The title compounds for Examples 2-6 are prepared by a method analogous to that described in Example 1.

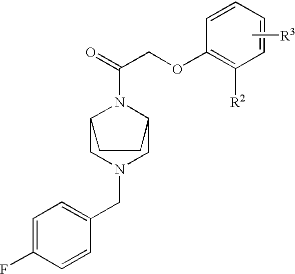

| Example | R³ | R² |
|---|---|---|
| 2 | 4-Cl | CO₂NH₂ |
| 3 | 3-Cl | CO₂Et |
| 4 | 4-Cl | COCH₃ |
| 5 | 4-Cl | SO₂NH₂ |
| 6 | 4-CF₃ | NO₂ |
| 7 | 4-Cl | CH₂CO₂CH₂CH₃ |

### EXAMPLE 8

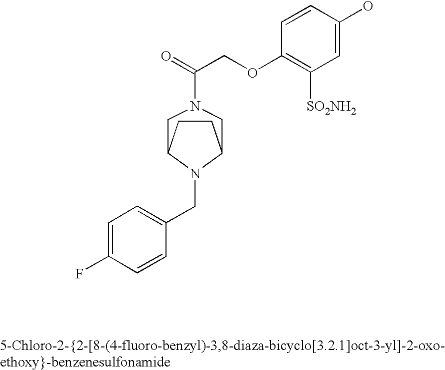

### 8-(4-Fluoro-benzyl)-3,8-diaza-bicyclo[3.2.1]octane

To a 0° C. solution of 8-(4-fluoro-benzyl)-3,8-diaza-bicyclo[3.2.1]octan-2-one (1.34 g, 5.72 mmol) in tetrahydrofuran (20 ml) is added lithium aluminum hydride (1.0 M in tetrahydrofuran, 11.4 ml, 11.4 mmol) dropwise. The reaction mixture is slowly warmed to ambient temperature and then refluxed for 3 hours. The resulting mixture is cooled to ambient temperature, quenched slowly with water and then filtered through a pad of celite. The filter cake is washed with ethyl acetate, and the combined organics are washed with saturated aqueous sodium hydrogen carbonate, dried over sodium sulfate, filtered and concentrated in vacuo to give the title compound (0.781 g, 62%).

The title compound for example 9-12 are prepared by a method analogous to that described in Example 8.

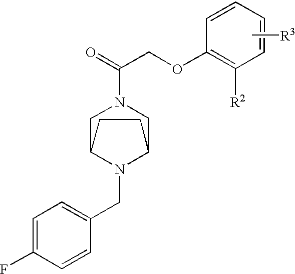

| Example | R³ | R² |
|---|---|---|
| 9 | 4-Cl | SO₂NH₂ |
| 10 | 4-Cl | CONH₂ |
| 11 | 3-OCH₃ | CONH₂ |
| 12 | 4-Cl | NO₂ |

### EXAMPLE 13

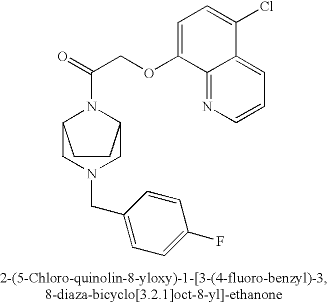

The title compound for Example 13 is prepared by a method analogous to that described in Example 1

### EXAMPLE 14

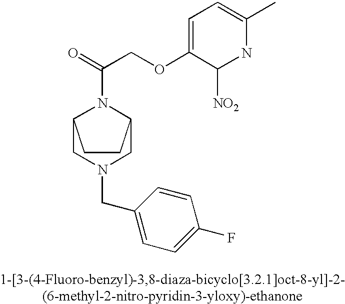

The title compound for Example 14 is prepared by a method analogous to that described in Example 1

### EXAMPLE 15

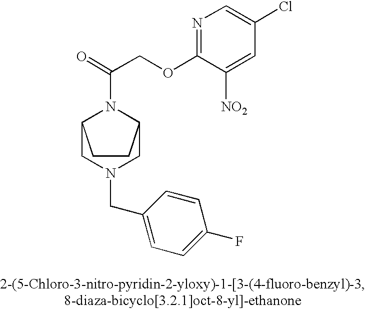

### Acetic Acid 2-[3-(4-fluoro-benzyl)-3,8-diaza-bicyclo[3.2.1]oct-8-yl]-2-oxo-ethyl Ester

To a solution of 3-(4-fluoro-benzyl)-3,8-diaza-bicyclo[3.2.1]octane (0.46g, 2.1 mmol) in dry dichloromethane (10 ml) at 0° C. is added triethylamine (0.32 ml, 2.3 mmol) followed by acetic acid chlorocarbonylmethyl ester (0.245 ml, 2.3 mmol). The resulting reaction mixture is stirred for 1 hour, then diluted with dichloromethane and washed with saturated aqueous sodium hydrogen carbonate. The aqueous layer is extracted three times with dichloromethane. The combined organics are dried over magnesium sulfate and concentrated in vacuo to give the title compound (0.67 g, 100%).

### 1-[3-(4-Fluoro-benzyl)-3,8-diaza-bicyclo[3.2.1]oct-8-yl]-2-hydroxy-ethanone

To a solution of acetic acid 2-[3-(4-fluoro-benzyl)-3,8-diaza-bicyclo[3.2.1]oct-8-yl]-2-oxo-ethyl ester (0.67 g, 2.1 mmol) in tetrahydrofuran (8 ml), methanol (1 ml), and water (1 ml) is added lithium hydroxide monohydrate (0.18 g, 4.2 mmol). The reaction mixture is stirred at ambient temperature overnight and then diluted with ethyl acetate and washed with saturated aqueous sodium hydrogen carbonate. The aqueous layer is extracted three times with ethyl acetate. The combined organics are dried over magnesium sulfate and concentrated in vacuo to give the title compound (0.48 g, 82%).

### 2-(5-Chloro-3-nitro-pyridin-2-yloxy)-1-[3-(4-fluoro-benzyl)-3,8-diaza-bicyclo[3.2.1]oct-8-yl]-ethanone

To a 0° C. solution of 1-[3-(4-fluoro-benzyl)-3,8-diaza-bicyclo[3.2.1]oct-8-yl]-2-hydroxy-ethanone (0.456 g, 1.64 mmol) in toluene (8.0 ml) is added sodium hydride (0.072 g. 1.80 mmol). The reaction mixture is stirred at 0° C. for 30 minutes. To the resulting reaction mixture is added 2,5-dichloro-3-nitro-pyridine (0.35 g. 1.80 mmol). The reaction mixture is heated to reflux for 2 hours, then cooled to ambient temperature, washed with saturated aqueous sodium hydrogen carbonate and brine. The aqueous layers are then extracted three times with ethyl acetate. The combined organics are dried over magnesium sulfate and concentrated in vacuo to give the title compound (0.76 g, 64%).

The title compounds for Examples 16-17 are prepared by a method analogous to that described in Example 15.

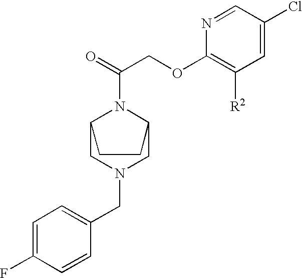

| Example | R² |
|---|---|
| 16 | CONH₂ |
| 17 | CO₂CH₃ |

### EXAMPLE 18

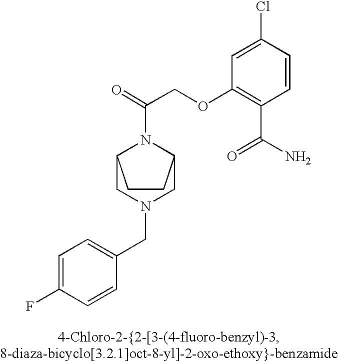

Ammonia gas is bubbled through a solution of 4-chloro-2-{2-[3-(4-fluoro-benzyl)-8-aza-bicyclo[3.2.1]oct-8-yl]-2-oxo-ethoxy}-benzoic acid methyl ester (0.030 g, 0.067 mmol) in dry methanol (2.0 ml). The reaction mixture is then capped and stirred at ambient temperature for two days, and then concentrated in vacuo. Silica gel chromatography gave the title compound (0.031 mg, 100%).

### EXAMPLE 19

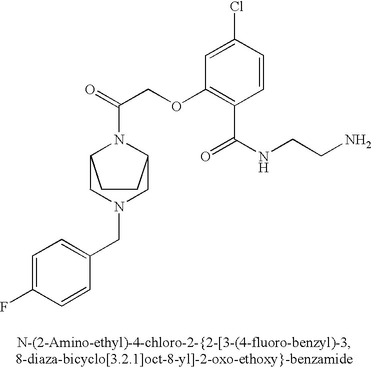

A solution of 4-chloro-2-{2-[3-(4-fluoro-benzyl)-3,8-diaza-bicyclo[3.2.1]oct-8-yl]-2-oxo-ethoxy}-benzoic acid methyl ester (0.160 g, 0.36 mmol) in ethane-1,2-diamine (12.0 ml) is heated to 450C overnight. The reaction mixture is then cooled to ambient temperature and concentrated in vacuo. Silica gel chromatography gave the title compound (0.13 g, 76%).

### EXAMPLE 20

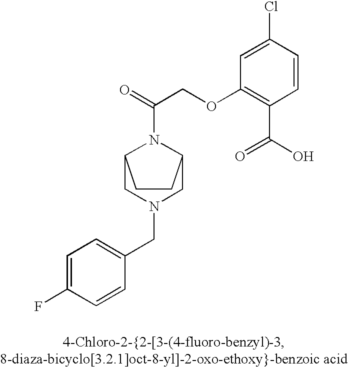

To a solution of of 4-chloro-2-{2-[3-(4-fluoro-benzyl)-3,8-diaza-bicyclo[3.2.1]oct-8-yl]-2-oxo-ethoxy}-benzoic acid methyl ester (0.080 g, 0.18 mmol) in a mixture of tetrahydrofuran (2 ml), methanol (0.2 ml) and water (0.4 ml) is added lithium hydroxide monohydrate (0.020 g, 0.36 mmol). The reaction is stirred at ambient temperature for 2 hours and then loaded on a silica gel column with methylene chloride. Silica gel chromatography gave the title compound (0.066 g, 78%).

The title compounds for Examples 21-25 are prepared by a method analogous to that described in Example 20.

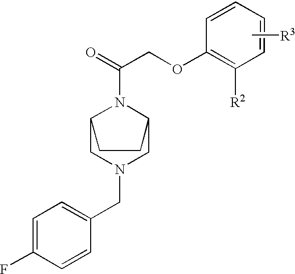

| Example | R³ | R² |
|---|---|---|
| 20 | 4-Cl | CO₂H |
| 21 | 4-CH₃ | CO₂H |
| 22 | 4-OCH₃ | CO₂H |
| 23 | 4-I | CO₂H |
| 24 | 4-Br | CO₂H |
| 25 | 4-Cl | CH₂CO₂H |

### EXAMPLE 26

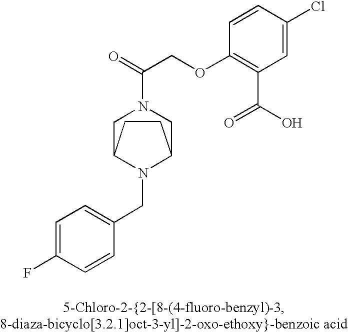

The title compound for Example 26 is prepared by a method analogous to that described in Example 20.

### EXAMPLE 27

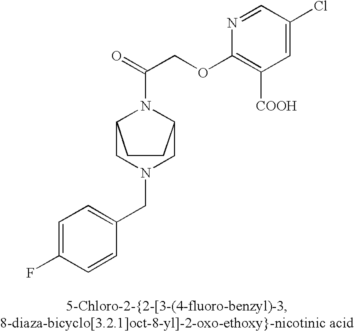

The title compound for Example 27 is prepared by a method analogous to that described in Example 20.

### EXAMPLE 28

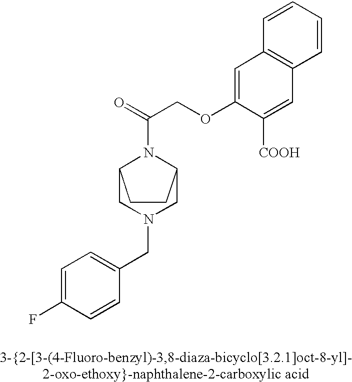

The title compound for Example 28 is prepared by a method analogous to that described in Example 20.

### EXAMPLE 29

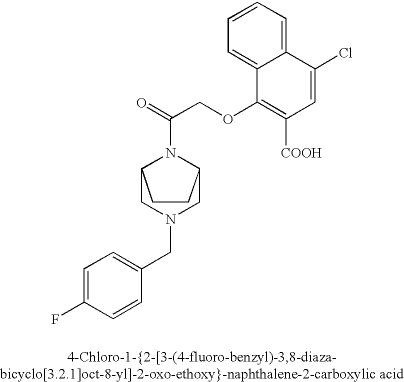

The title compound for Example 29 is prepared by a method analogous to that described in Example 20.

### EXAMPLE 30

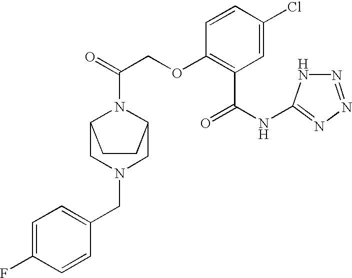

To a solution of 5-chloro-2-{2-[3-(4-fluoro-benzyl)-3,8-diaza-bicyclo[3.2.1]oct-8-yl]-2-oxo-ethoxy}-benzoic acid (0.090 g, 0.21 mmol) in tetrahydrofuran (2.0 ml) is added carbonyl diimidazole (0.037 g, 0.23 mmol). The reaction mixture is refluxed for 3 hours and then cooled to ambient temperature. To the resulting reaction mixture is added 1H-tetrazol-5-ylamine (0.0195 g, 0.23 mmol). The resulting mixture is heated to reflux for 12 hours. The reaction is then cooled, diluted with dichloromethane and washed with saturated aqueous sodium hydrogen carbonate. The organic layers are dried over magnesium sulfate, filtered and concentrated in vacuo. Silica gel chromatography gave the title compound (0.10 g, 95%).

The title compounds for Examples 31-32 are prepared by a method analogous to that described in Example 30.

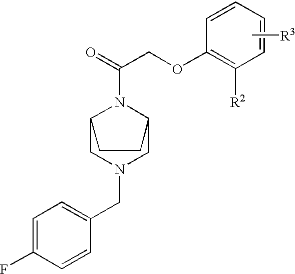

| Example | R³ | R² |
|---|---|---|
| 31 | 4-Cl | CONHCH₂CO₂H |
| 32 | 4-Cl | CONHSO₂CH₃ |

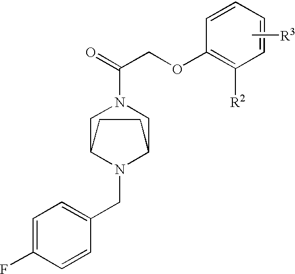

| Example | R³ | R² |
|---|---|---|
| 33 | 4-Cl | CONHTet |
| 34 | 4-Cl | CONHCH₂CONH₂ |

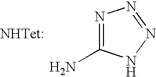

### EXAMPLE 35

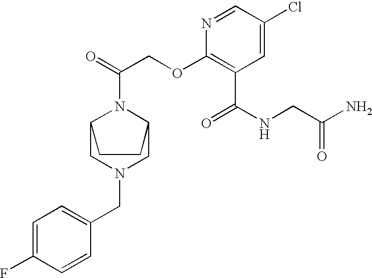 N-Carbamoylmethyl-5-chloro-2-{2-[3-(4-fluoro-benzyl)-
3,8-diaza-bicyclo[3.2.1]oct-8-
yl]-2-oxo-ethoxy}-nicotinamide

The title compound for Examples 35 is prepared by a method analogous to that described in Example 30.

### EXAMPLE 36

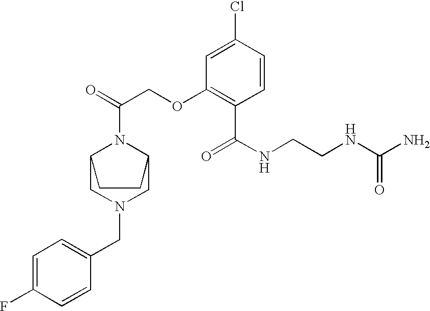 4-Chloro-2-{2-[3-(4-fluoro-benzyl)-3,8-diaza-
bicyclo[3.2.1]oct-8-yl]-2-oxo-ethoxy}-N-
(2-ureido-ethyl)-benzamide

To a solution of N-(2-amino-ethyl)-4-chloro-2-{2-[3-(4-fluoro-benzyl)-3,8-diaza-bicyclo[3.2.1]oct-8-yl]-2-oxo-ethoxy}-benzamide (0.065 g, 0.14 mmol) in dichloromethane (1.5 ml) is added pyridine (0.023 ml, 0.28 mmol) and 4-nitrophenyl chloroformate (0.028 g, 0.14 mmol). The reaction is stirred at ambient temperature for 1 hour and then concentrated in vacuo. The resulting residue is dissolved in methanol, treated with ammonia gas, and the solution is stirred under an atmosphere of ammonia over night. The reaction mixture is diluted with ethyl acetate, washed with saturated aqueous sodium hydrogen carbonate and 1M aqueous sodium hydroxide until the organic layer is colorless. Silica gel chromatography gave the title compound (0.057 mg, 79%).

The title compounds for Examples 37-39 are prepared by a method analogous to that described in Example 36.

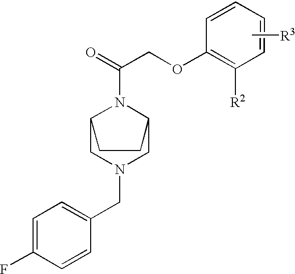

| Example | R³ | R² |
|---|---|---|
| 37 | 3-Cl | NHCONHCH₂CONH₂ |
| 38 | 4-Cl | NHCONH₂ |
| 39 | 4-Cl | NHCONHCH₂CH₂CO₂H |

### EXAMPLE 40

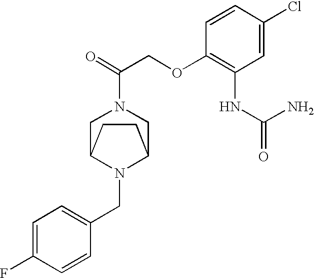

The title compound for Example 40 is prepared by a method analogous to that described in Example 36.

### EXAMPLE 41

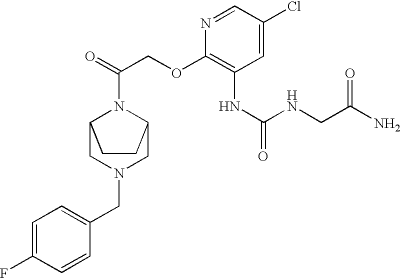

The title compound for Example 41 is prepared by a method analogous to that described in Example 36.

### EXAMPLE 42

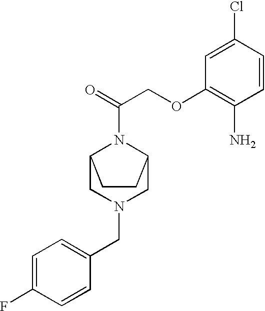

To a solution of 2-(5-chloro-2-nitro-phenoxy)-1-[3-(4-fluoro-benzyl)-3,8-diaza-bicyclo[3.2.1]oct-8-yl]-ethanone (0.059 g, 0.136 mmol) in ethanol (15 ml) in a par bottle is added platinum on carbon (5%, 0.100 g). The resulting suspension is subjected to hydrogen gas (20 psi) for 10 minutes, filtered through a pad of celite, the filter cake is washed with ethyl acetate. The filtrate is concentrated in vacuo to give the title compound (0.040 g, 74%).

The title compounds for Examples 43-44 are prepared by a method analogous to that described in Example 42.

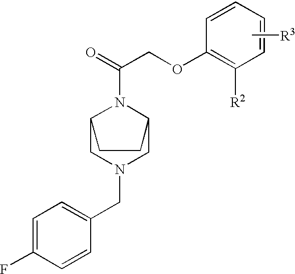

| Example | R³ | R² |
|---|---|---|
| 43 | 4-CF₃ | NH₂ |
| 44 | 4-Cl | NH₂ |

### EXAMPLE 45

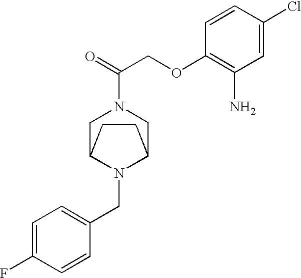

The title compound for Examples 45 is prepared by a method analogous to that described in Example 42.

### EXAMPLE 46

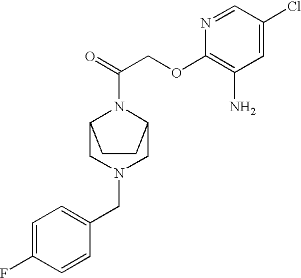

The title compound for Examples 46 is prepared by a method analogous to that described in Example 42.

### EXAMPLE 47

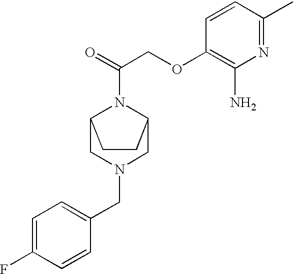

The title compound for Examples 47 is prepared by a method analogous to that described in Example 42.

### EXAMPLE 48

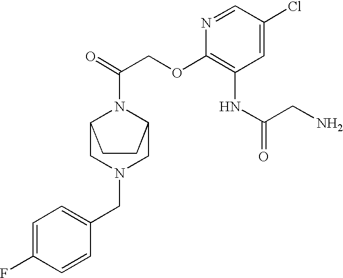

To a solution of tert-butoxycarbonylamino-acetic acid (0.061 g, 0.35 mmol) in tetrahydrofuran (2.0 ml) at 0 C. is added N-methyl morpholine (0.038 ml, 0.35 mmol) and isobutylchloroformate (0.045 ml, 0.35 mmol). The reaction mixture is stirred for 20 minutes, warmed to ambient temperature for 2 hours, and then treated with 2-(3-amino-5-chloro-pyridin-2-yloxy)-1-[3-(4-fluoro-benzyl)-3,8-diaza-bicyclo[3.2.1]oct-8-yl]-ethanone (0.109 g, 0.26 mmol) in tetrahydrofuran (1.0 ml). The resulting mixture is stirred over night at ambient temperature and then filtered through a pad of celite, which is washed with tetrahydrofuran. The filtrate is diluted with ethyl acetate and washed with brine. The organic layer is dried over magnesium sulfate, filtered and concentrated in vacuo. Silica gel chromatography gave the BOC-protected title compound (0.092 g, 63%), which is then treated with trifluoroacetic acid (1.0 ml) in dichloromethane (10 ml). The reaction mixture is stirred at ambient temperature overnight, diluted with dichloromethane, washed with aqueous sodium hydroxide (1N, 10.0 ml), dried over magnesium sulfate, filtered and concentrated in vacuo to give the title compound (0.054 g, 73%).

### EXAMPLE 49

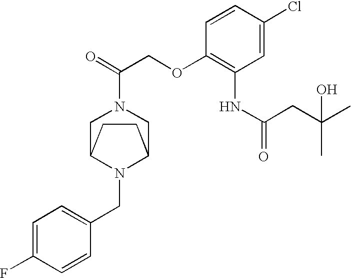

The title compound for Example 49 is prepared by a method analogous to that described in Example 48.

### EXAMPLE 50

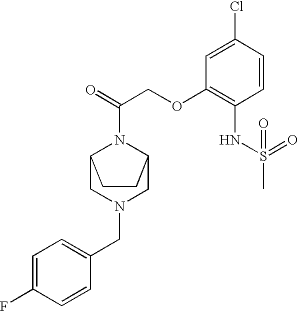

To a 0° C. solution of 2-(2-amino-5-chloro-phenoxy)-1-[3-(4fluoro-benzyl)-3,8-diaza-bicyclo[3.2.1]oct-8-yl]-ethanone (0.040 g, 0.10 mmol) in dichloromethane (1.0 ml) is added triethylamine (0.028 ml, 0.20 mmol) and methanesulfonyl chloride (0.010 ml, 0.012 mmol). The reaction mixture is stirred at 0° C. for 30 minutes and then concentrated in vacuo. Silica gel chromatography gave the title compound (0.025 mg, 52%)

The title compounds for Examples 51-53 are prepared by a method analogous to that described in Example 50

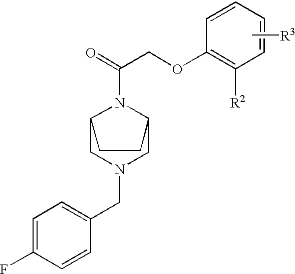

| Example | R³ | R² |
|---|---|---|
| 51 | 4-CF₃ | NHSO₂CH₃ |
| 52 | 4-Cl | NHSO₂CH₃ |
| 53 | 3-Cl | CONHCH₂CH₂NHSO₂CH₃ |

### EXAMPLE 54

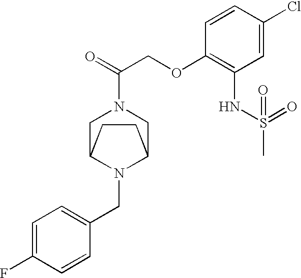

The title compound for Example 54 is prepared by a method analogous to that described in Example 50.

### EXAMPLE 55

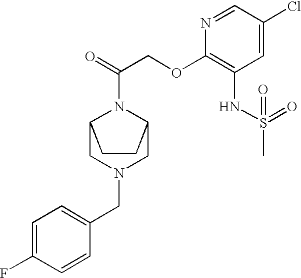

The title compound for Examples 55 is prepared by a method analogous to that described in Example 50.

### EXAMPLE 56

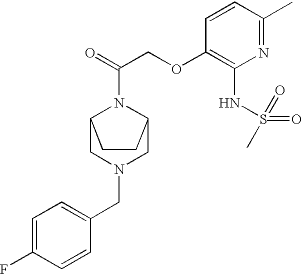

The title compound for Examples 56 is prepared by a method analogous to that described in Example 50.

### EXAMPLE 57

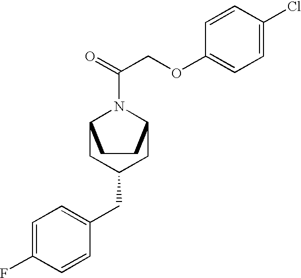

A solution of 2,5-dimethoxy-tetrahydrofuran (30 g, 0.23 mmol) in aqueous hydrochloric acid (0.25 M, 100 ml) is stirred at 0° C. overnight. To the resulting reaction mixture is then added benzylamine hydrochloride salt (39.9 g, 0.27 mmol), 3-oxo-pentanedioic acid (33.6 g, 0.23 mmol) and aqueous sodium acetate (2.75 M, 200 ml, 0.55 mmol). The resulting reaction mixture is stirred at ambient temperature for 90 minutes, and then heated to 50° C. for 2 hours. The reaction mixture is then cooled to 0° C. and basified with 6N aqueous sodium hydroxide (50 ml) to pH =10. The reaction is extracted with ethyl acetate, the organic layers are dried over magnesium sulfate, filtered and concentrated in vacuo. Silica gel chromatography gave the title compound (37.54 g, 75%).

### 3-Oxo-8-aza-bicyclo[3.2.1]octane-8-carboxylic Acid Tert-Butyl Ester

To a solution of 8-benzyl-8-aza-bicyclo[3.2.1]octan-3-one (33 g, 0.153 mmol) in ethyl acetate (100 ml) in a par bottle is added di-tert-butyl-dicarbonate (40.15 g, 0.184 mmol) and palladium hydroxide on carbon (20%, 20 g). The reaction mixture is subjected to hydrogen gas (50 psi) at ambient temperature for 5 hours, filtered through a pad of celite, and the filter cake is washed with ethyl acetate. The filtrate is concentrated in vacuo and silica gel chromatography gave the title compound (29.37 g, 85%).

### 3-(4-Fluoro-benzylidene)-8-aza-bicyclo[3.2.1]octane-8-carboxylic Acid Tert-Butyl Ester

To a solution of (4-fluoro-benzyl)-triphenyl-phosphonium chloride (27.0 g, 66.5 mmol) in toluene (500 ml) is added sodium hydride (60% dispersion, 2.66 g, 66.5 mmol). The resulting suspension is stirred at ambient temperature for 90 minutes. To the reaction mixture is then added 3-oxo-8-aza-bicyclo[3.2.1]octane-8-carboxylic acid tert-butyl ester (13.6 g, 60.5 mmol). The resulting reaction mixture is refluxed overnight, and then cooled to ambient temperature, diluted with water and extracted with diethyl ether. The organic layers are dried over magnesium sulfate, filtered and concentrated in vacuo. Silica gel chromatography gave the title compound (17.61 g, 92%).

### 3-(4-Fluoro-benzyl)-8-aza-bicyclo[3.2.1]octane-8-carboxylic Acid Tert-Butyl Ester

To a solution 3-(4-fluoro-benzylidene)-8-aza-bicyclo[3.2.1]octane-8-carboxylic acid tert-butyl ester (18.72 g, 59.0 mmol) in ethanol (500 ml) in a par bottle is added palladium on carbon (10%, 10.0 g). The reaction mixture is subjected to hydrogen gas (40 psi) for 2 hours. The reaction mixture is filtered through a pad of celite, and the filter cake is washed with ethanol. The filtrate is concentrated in vacuo to give the title compound (18.12 g, 96%, 2:1 mixture of diastereomers by 1 H NMR). Chiral HPLC separation gave the exo diastereomer (4.24 g, 23%) along with the endo diastereomer (11.32 g, 60%).

### 3-(4-Fluoro-benzyl)-8-aza-bicycelo[3.2.1]octane

To a solution of 3-(4-fluoro-benzyl)-8-aza-bicyclo[3.2.1]octane-8-carboxylic acid tert-butyl ester (4.24 g, 13.3 mmol) in dichloromethane (50 ml) is added trifluoroacetic acid (10 ml). The resulting reaction mixture is stirred at ambient temperature for 3 hours, then washed with 1N aqueous sodium hydroxide, and extracted with dichloromethane three times. The combined organics are dried over magnesium sulfate, filtered and concentrated in vacuo to give the title compound (2.91 g,100%).

### 2-(4-Chloro-phenoxy)-1-r3-(4-fluoro-benzyl)-8-aza-bicyclo[3.2.1]oct-8-yl]-ethanone

To a solution of 3-(4-fluoro-benzyl)-8-aza-bicyclo[3.2.1]octane (0.038 g, 0.173 mmol) in dichloromethane (1 ml) at 0° C. is added (4-chloro-phenoxy)-acetyl chloride (0.042 g, 0.208 mmol) and triethylamine (0.072 ml, 0.520 mmol). The reaction mixture is slowly warmed to ambient temperature, and then diluted with dichloromethane and washed with water. The organics are dried over magnesium sulfate, filtered and concentrated in vacuo. Silica gel chromatography gave the title compound (0.042 g, 63%).

### EXAMPLE 58

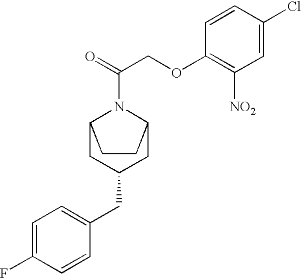

### 2-chloro-1-[3-(4-fluoro-benzyl )-8-aza-bicyclo[3.2.1]octane]-ethanone

To a solution of 3-(4-fluoro-benzyl)-8-aza-bicyclo[3.2.1]octane (2.91 g, 13.28 mmol) in dry dichloromethane (30 ml) at 0° C. is added triethylamine (2.10 ml, 14.60 mmol) followed by chloroacetyl chloride (1.10 ml, 14.60 mmol). The resulting reaction mixture is stirred for 60 minutes, diluted with dichloromethane and washed with saturated aqueous sodium hydrogen carbonate. The organics are dried over magnesium sulfate, filtered and concentrated in vacuo. Silica gel chromatography gave the title compound (3.58 g, 91%).

### 2-(4-Chloro-2-nitro-phenoxy)-1-[3-(4-fluoro-benzyl)-8-aza-bicyclo[3.2.1]oct-8-yl]-ethanone

To a solution of 2-chloro-1-[3-(4-fluoro-benzyl)-8-aza-bicyclo[3.2.1]octane]-ethanone (1.0 g, 3.40 mmol) in butanone (7 ml) is added 2-nitro-4-trifluoromethyl-phenol (0.65 g, 3.74 mmol), potassium carbonate (0.93 g, 6.8 mmol) and potassium iodide (0.56 g, 3.40 mmol). The resulting mixture is stirred at 60° C. for 24 hours. The reaction is then cooled, diluted with ethyl acetate and washed with brine. The organic layers are dried over magnesium sulfate, filtered and concentrated in vacuo. Silica gel chromatography gave the title compound (1.3 g, 88%)

The title compounds for Examples 59-65 are prepared by a method analogous to that described in Example 58

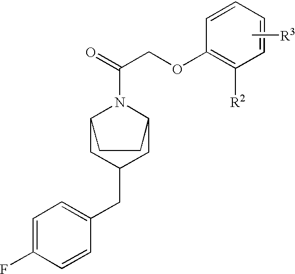

| Example | R³ | R² |
|---|---|---|
| 59 | 4-Cl | CO₂CH₃ |
| 60 | 4-Cl | COCH₃ |
| 61 | 4-Cl | SO₂NH₂ |
| 62 | 4-Cl | CH₂NPhth |
| 63 | 4-CF₃ | NO₂ |
| 64 | 3-Cl | CO₂CH₃ |
| 65 | 3-CH₃ | NHCOCH₃ |

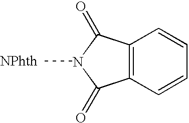

### EXAMPLE 66

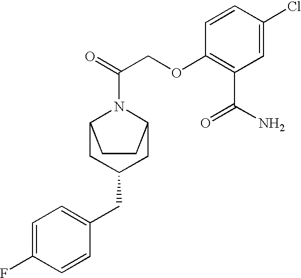

Ammonia gas is bubbled through a solution of 5-chloro-2-{2-[3-(4-fluoro-benzyl)-8-aza-bicyclo[3.2.1]oct-8-yl]-2-oxo-ethoxy}-benzoic acid methyl ester (0.015 g, 0.034 mmol) in dry methanol (3.0 ml). The reaction mixture is then capped and stirred at ambient temperature for two days, and then concentrated in vacuo. Silica gel chromatography gave the title compound (0.0008 mg, 55%).

### EXAMPLE 67

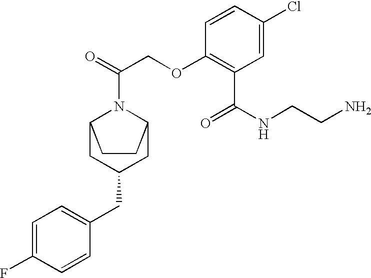

A solution of 5-chloro-2-{2-[3-(4-fluoro-benzyl)-8-aza-bicyclo[3.2.1]oct-8-yl]-2-oxo-ethoxy}-benzoic acid methyl ester (0.015 g, 0.034 mmol) in ethane-1,2-diamine (2.0 ml) is heated to 45° C. overnight. The reaction mixture is then cooled to ambient temperature and concentrated in vacuo. Silica gel chromatography gave the title compound (0.011 g, 69%).

### EXAMPLE 68

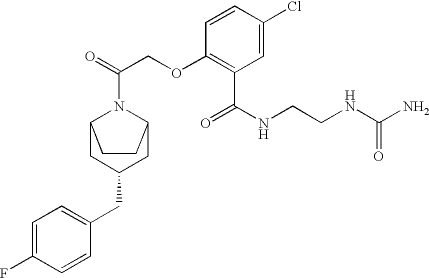

To a solution of N-(2-Amino-ethyl)-5-chloro-2-{2-[3-(4-fluoro-benzyl)-8-aza-bicyclo[3.2.1]oct-8-yl]-2-oxo-ethoxy}-benzamide (0.104 g, 0.22 mmol) in dichloromethane (2 ml) is added pyridine (0.035 ml, 0.44 mmol) and 4-nitrophenyl chloroformate (0.048 g, 0.24 mmol). The reaction is stirred at ambient temperature for 1 hour and then concentrated in vacuo. The resulting residue is dissolved in methanol, ammonia gas is bubbled through the reaction mixture, and the solution is stirred under an atmosphere of ammonia overnight. The reaction mixture is concentrated in vacuo and silica gel chromatography gave the title compound (0.049 mg, 43%).

The title compounds for Examples 69-71 are prepared by a method analogous to that described in Example 68.

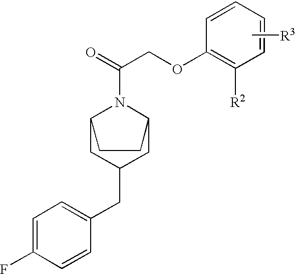

| Example | R³ | R² |
|---|---|---|
| 69 | 4-Cl | NHCONH₂ |
| 70 | 4-CF₃ | NHCONH₂ |
| 71 | 3-Cl | NHCONH₂ |

Example 72
2-(2-Amino-4-chloro-phenoxy)-1-[3-(4-fluoro-benzyl)-8-aza-bicyclo[3.2.1]oct-8-yl]-ethanone

To a solution of 2-(4-chloro-2-nitro-phenoxy)-1-[3-(4-fluoro-benzyl)-8-aza-bicyclo[3.2.1]oct-8-yl]-ethanone (1.23 g, 2.84 mmol) in ethanol (50 ml) in a par bottle is added platinum on carbon (5%, 0.500 g). The resulting suspension is subjected to hydrogen gas (35 psi) for 3 hours, filtered through a pad of celite, the filter cake is washed with ethyl acetate. The filtrate is concentrated in vacuo to give the title compound (0.95 g, 83%).

The title compounds for Examples 73-74 are prepared by a method analogous to that described in Example 72.

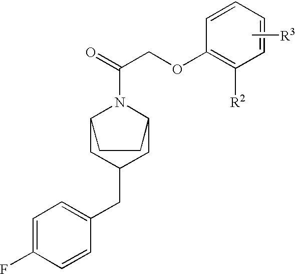

| Example | R³ | R² |
|---|---|---|
| 73 | 4-CF₃ | NH₂ |
| 74 | 3-Cl | NH₂ |

### EXAMPLE 75

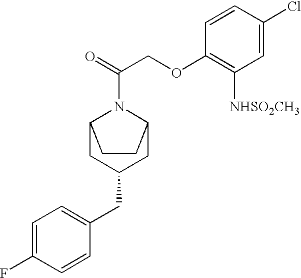

To a solution of 2-(2-amino-4-chloro-phenoxy)-1-[3-(4-fluoro-benzyl)-8-aza-bicyclo[3.2.1]oct-8-yl]-ethanone (0.050 g, 0.13 mmol) in dichloromethane (1.0 ml) is added triethylamine (0.036 ml, 0.26 mmol) and methanesulfonyl chloride (0.011 ml, 0.014 mmol). The reaction mixture is stirred at ambient temperature for 1 hour and then concentrated in vacuo. Silica gel chromatography gave the title compound (0.034 mg, 54%)

The title compounds for Examples 76-81 are prepared by a method analogous to that described in Example 75.

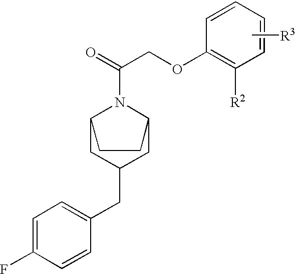

| Example | R³ | R² |
|---|---|---|
| 76 | 4-CF₃ | NHSO₂CH₃ |
| 77 | 3-Cl | NHSO₂CH₃ |
| 78 | 3-Cl | N(SO₂CH₃)₂ |
| 79 | 3-Cl | NHCH₂CH₂NHSO₂CH₃ |
| 80 | 4-Cl | NHCH₂CH₂NHSO₂CH₃ |
| 81 | 4-Cl | NHSO₂CF₃ |

### EXAMPLE 82

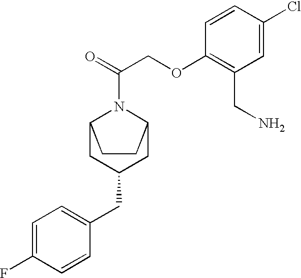

To a solution of 2-(5-chloro-2-{2-[3-(4-fluoro-benzyl)-8-aza-bicyclo[3.2.1]oct-8-yl]-2-oxo-ethoxy}-benzyl)-isoindole-1,3-dione (1.46 g, 2.67 mmol) in ethanol (30 ml) is added hydrazine (35%, 5.0 ml, 54 mmol). The reaction mixture is stirred at ambient temperature overnight. The reaction mixture is then filtered through a glass frit, the white precipitate is washed with ethanol, and the combined filtrates are concentrated in vacuo. The resulting residue is triterated with dichloromethane and filtered through a glass frit. The filtrate is concentrated in vacuo to give the title compound (1.06 g, 95.4%).

### EXAMPLE 83

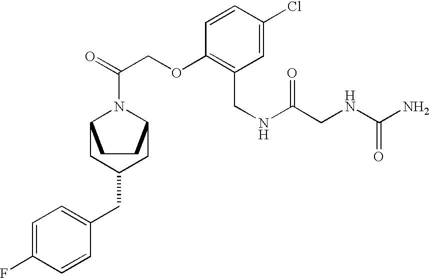

To a solution of 2-(2-aminomethyl-4-chloro-phenoxy)-2-[3-(4-fluoro-benzyl)-8-aza-bicyclo[3.2.1]oct-8-yl]-ethanone (0.060 g, 0.14 mmol) in dichloromethane (1.5 ml) is added triethylamine (0.036 mg, 0.36 mmol), tert-butyl 1-piperazinecarboxylate (0.021 g, 0.1a5 mmol), 1-(3-dimethylaminopropyl)-3-ethylcarbodiimide hydrochloride (0.034 g, 0.15 mmol) and ureido-acetic acid (0.017 g, 0.15 mmol). The resulting reaction mixture is stirred at ambient temperature overnight, then diluted with dichloromethane and washed with saturated aqueous sodium hydrogen carbonate. The organics are dried over magnesium sulfate, filtered and concentrated in vacuo. Silica gel chromatography gave the title compound (0.034 g, 47%).

### EXAMPLE 84

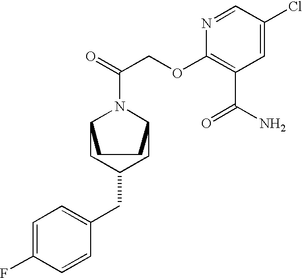 5-Chloro-2-{2-[3-(4-fluoro-benzyl)-8-aza-
bicyclo[3.2.1]oct-8-yl]-2-oxo-ethoxy}-
nicotinamide

To a solution of glycolic acid (0.157 g, 2.07 mmol), dimethylamino pyridine (catalytic), and pyridine (0.327g, 4.14 mmol), in dichloromethane (6 ml) is added dropwise chloro trimethylsilane (0.526 ml, 4.14 mmol). The reaction mixture is stirred at ambient temperature for 4 hours, and then catalytic dimethylformamide and oxalyl chloride (2 M in dichloromethane, 1.0 ml, 2.0 mmol) are added. The resulting reaction mixture is stirred at 0° C. for 1 hour, warmed to ambient temperature for 30 minutes, and then cooled to 0° C. To the mixture is added (4-fluoro-benzyl)-8-aza-bicyclo[3.2.1]octane (0.500 g, 2.2 mmol) in pyridine (0.474 g, 6.1 mmol). The reaction mixture is then slowly warmed to ambient temperature over 2 hours. Citric acid (0.422 g, 2.2 mmol) in methanol (6.0 ml) is added to the resulting reaction mixture, which is then stirred for 30 minutes at ambient temperature. The reaction mixture is diluted with ethyl acetate, washed with IN aqueous hydrochloric acid, then saturated aqueous sodium hydrogen carbonate and brine. The organics are dried over magnesium sulfate, filtered and concentrated in vacuo to give 1-[3-(4-fluoro-benzyl)-8-aza-bicyclo[3.2.1]oct-8-yl]-2-hydroxy-ethanone (0.480 g, 84%).

To a 0° C. solution of 1-[3-(4-fluoro-benzyl)-8-aza-bicyclo[3.2.1]oct-8-yl]-2-hydroxy-ethanone (0.075 g, 0.27 mmol) in toluene (2.0 ml) is added sodium hydride (0.012 g, 0.29 mmol), and the reaction mixture is stirred at 0° C. for 30 minutes. To the reaction mixture is then added 2,5-dichloro-nicotinamide (0.056 g, 0.29 mmol). The resulting mixture is refluxed for 2 hours, cooled to ambient temperature and concentrated in vacuo. Silica gel chromatography gave the title compound (0.070 g, 60%).

### EXAMPLE 85

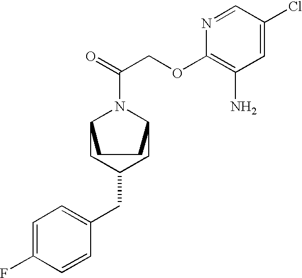 2-(3-Amino-5-chloro-pyridin-2-yloxy)-1-[3-(4-fluoro-
benzyl)-8-aza-bicyclo[3.2.1]oct-8-
yl]-ethanone

To a 0° C. solution of 1-[3-(4-fluoro-benzyl)-8-aza-bicyclo[3.2.1]oct-8-yl]-2-hydroxy-ethanone (0.253 g, 0.913 mmol) in toluene (2.0 ml) is added sodium hydride (0.042 g, 1.1 mmol), and the reaction mixture is stirred at 0° C. for 30 minutes. To the reaction mixture is then 2,5-dichloro-3-nitro-pyridine (0.185 g, 0.98 mmol). The resulting mixture is refluxed for 2 hours, cooled to ambient temperature and concentrated in vacuo. Silica gel chromatography gave 2-(3-nitro-5-chloro-pyridin-2-yloxy)-l-[3-(4-fluoro-benzyl)-8-aza-bicyclo[3.2.1]oct-8-yl]-ethanone (0.272 g, 68%).

To a solution of 2-(3-nitro-5-chloro-pyridin-2-yloxy)-1-[3-(4-fluoro-benzyl )-8-aza-bicyclo[3.2.1]oct-8-yl]-ethanone (0.269 g, 0.621 mmol) in ethanol (10 ml) in par bottle is added platinum dioxide (0.250 g). The reaction mixture is subjected to hydrogen gas (35 psi) for 20 minutes, then filtered through a pad of celite, the filter cake is washed with ethanol, and the filtrate is concentrated in vacuo. Silica gel chromatography gave the title compound (0.135 g, 54%).

### EXAMPLE 86

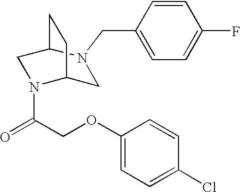

To a solution of 2,5-dibromo-hexanedioic acid diethyl ester (5.0 g, 0.013 mmol) in ethanol (16 ml) is added sodium azide (2.4 g, 0.036 mmol). The reaction mixture is refluxed overnight and then poured slowly into ice water. The product is extracted with diethyl ether three times; the organics are dried over magnesium sulfate, filtered and concentrated in vacuo to give crude product, which is dissolved in ethanol (75 ml) and concentrated aqueous hydrochloric acid (5.5 ml). To the reaction mixture is added platinum oxide (1.1 g). The reaction mixture is shaken under an atmosphere of hydrogen (30 psi) overnight, then filtered through a pad of celite, and the filter cake is washed with ethanol. The filtrate is concentrated in vacuo to give the title compound (5.6 g, 100%).

### 2,5-Diaza-bicyclo[2.2.2]octane Hydrochloride Salt

To a solution of 2,5-diamino-hexanedioic acid diethyl ester hydrochloride salt (5.6 g, 0.013 mmol) in methanol (400 ml) is added sodium methoxide (2.78 g, 0.051 mmol) giving a solution with pH=14. The reaction mixture is refluxed overnight, and then concentrated in vacuo. The resulting residue is washed twice with boiling ethanol. The filtrate is concentrated in vacuo to give the crude product, which is dissolved in tetrahydrofuran (100 ml) and treated with lithium aluminum hydride (1.0M in tetrahydrofuran, 80 ml, 0.080 mmol) at 0° C. The reaction mixture is slowly warmed to ambient temperature and then refluxed overnight, cooled to 0° C. and slowly quenched with water. The resulting mixture is filtered through a pad of celite, and the filter cake is washed with diethyl ether and dichloromethane. The filtrate is treated with hydrochloric acid and then concentrated in vacuo to give the title compound (0.30 g, 16% over 2 steps)

### 2-(4-Fluoro-benzyl)-2,5-diaza-bicyclo[2.2.2]octane

To a solution of 2,5-diaza-bicyclo[2.2.2]octane hydrochloride salt (0.30 g, 1.60 mmol) in 1,2-dichloroethane (3.2 ml) is added 4-fluoro-benzaldehyde (0.043 ml, 0.40 mmol), triethylamine (0.5 ml, 13.6 mmol) and acetic acid (0.3 ml). The reaction mixture is stirred for 2 hours, and then treated with sodium triacetoxy borohydride (0.14 g, 2.72 mmol) and stirred at ambient temperature overnight. The reaction mixture is treated with water and extracted with ethyl acetate. The organics are dried over magnesium sulfate, filtered and concentrated in vacuo to give the title compound contaminated with triethylammonium acetate (0.44g, 66% by NMR).

### 2-(4-Chloro-phenoxy)-1-[5-(4-fluoro-benzyl)-2,5-diaza-bicyclo[2.2.2]oct-2-yl]-ethanone

To a solution of 2-(4-fluoro-benzyl)-2,5-diaza-bicyclo[2.2.2]octane (0.026 mg, 0.12 mmol) in dichloroethane (1 ml) at 0° C. is added (4-chloro-phenoxy)-acetyl chloride (28 mg, 0.13 mmol). The reaction mixture is slowly warmed to ambient temperature, and then quenched with saturated aqueous sodium hydrogen carbonate. The product is extracted with ethyl acetate, the organics are washed with brine, dried over magnesium sulfate, filtered and concentrated in vacuo to give the title compound (0.023 mg, 76%).

### EXAMPLE 87

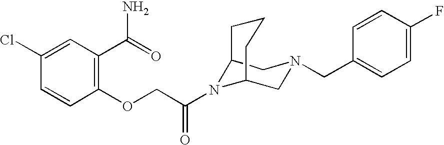

To a solution of pyridine-2,6-dicarboxylic acid (20.0 g, 0.119 mol) in 2M aqueous sodium hydroxide (150 ml) in a par bottle is added rhodium on aluminum (5%, 1.49 g). The resulting suspension is subjected to hydrogen gas (50 psi) for 72 hours, filtered through a pad of celite, the filter cake is washed with water. The filtrate is cooled to 0° C. and treated with benzyl chloro formate (24.2 g, 0.142 mol) in tetrahydrofuran (100 ml). The reaction mixture is stirred at ambient temperature for 5 hours. The reaction mixture is extracted with diethyl ether, the aqueous layer is acidified with 6N aqueous hydrochloric acid and then extracted with ethylacetate. The combined organics are dried over magnesium sulfate, filtered and concentrated in vacuo. Tritration with ethylacetate gave the title compound (18.0 g, 48%).

### 3-(4-Fluoro-benzyl)-2,4-dioxo-3.9-diaza-bicyclo[3.3.1]nonane-9-carboxylic Acid Benzyl Ester

A solution of piperidine-1,2,6-tricarboxylic acid 1-benzyl ester (18.0 g, 0.059 mol) in acetic anhydride (200 ml) is heated to 70° C. overnight. The reaction mixture is cooled to ambient temperature, concentrated in vacuo, and the residue is azeotroped with toluene. The resulting oil is dissolved in toluene (200 ml) and treated with 4-fluorobenzyl amine (7.3 g, 0.059 mol). The reaction mixture is stirred at ambient temperature for 18 hours and then treated with acetic anhydride (20 ml) and heated to reflux for 16 hours. The reaction mixture is cooled to 0° C., poored into a mixture of saturated aqueous sodium hydrogen carbonate and crushed ice, and extracted with ethyl acetate. The combined organics are dried over magnesium sulfate, filtered and concentrated in vacuo. Silica gel chromatography gave the title compound (19.72 g, 84%)

### 3-(4-Fluoro-benzyl)-3.9-diaza-bicyclo[3.3.1]nonane-2,4-dione

To a solution of 3-(4-fluoro-benzyl)-2,4-dioxo-3,9-diaza-bicyclo[3.3.1]nonane-9-carboxylic acid benzyl ester (9.37 g, 0.023 mol) in ethanol (100 ml) is added cyclohexadiene (18.9 g, 0.23 mol) and palladium on carbon (10%, 5.0 g). The reaction mixture is stirred at ambient temperature for 90 minutes, then filtered through a pad of celite, the filter cake is washed with ethanol. The filtrate is concentrated to give the title compound (5.69 g, 94%).

### 3-(4-Fluoro-benzyl)-3,9-diaza-bicyclo[3.3.1]nonane

To a solution 0° C. of 3-(4-fluoro-benzyl)-3,9-diaza-bicyclo[3.3.1]nonane-2,4-dione (5.69 g, 0.0217 mol) in toluene (70 ml) is added Red-Al (20 ml, 0.100 mol). The reaction mixture is warmed to 60° C. for 4 hours, and then cooled to 0° C. and treated with water (50 ml), 1N aqueous sodium hydroxide (50 ml) and saturated aqueous ammonium chloride. The resulting mixture is filtered through a pad of celite, extracted with ethylacetate, and the combined organics are dried over magnesium sulfate, filtered and concentrated in vacuo to give the title compound (4.51 g, 88%).

### 2-chloro-1-[3-(4-fluoro-benzyl)-3,9-diaza-bicyclo[3.9.1]non-8-yl]-ethanone

To a solution of 3-(4-fluoro-benzyl)-3,9-diaza-bicyclo[3.9.1]nonane (0.65g, 2.77 mmol) in dry dichloromethane (6 ml) at 0° C. is added triethylamine (0.43 ml, 3.10 mmol) followed by choroacetyl chloride (0.23 ml, 3.10 mmol). The resulting reaction mixture is stirred for two hours and concentrated in vacuo. Silica gel chromatography gave the title compound (0.53 g, 61%).

### 5-Chloro-2-{2-[3-(4-fluoro-benzyl)-3.9-diaza-bicyclo[3.3.1]non-9-yl]-2-oxo-ethoxy}-benzamide

To a solution of 2-chloro-1-[3-(4-fluoro-benzyl)-3,9-diaza-bicyclo[3.3.1]oct-8-yl]-ethanone (0.095g, 0.30 mmol) in butanone (4 ml) is added 2-hydroxy-5-chloro-benzyamide (0.0.057 g, 0.33 mmol), potassium carbonate (0.082 g, 0.60 mmol) and potassium iodide (0.0.049 g, 0.30 mmol). The resulting mixture is stirred at reflux for 7 hours. The reaction is then cooled, diluted with ethyl acetate and washed with brine. The organic layers are dried over magnesium sulfate, filtered and concentrated in vacuo. Silica gel chromatography gave the title compound (0.090 g, 67%).

The title compounds for Examples 88-92 are prepared by a method analogous to that described in Example 87.

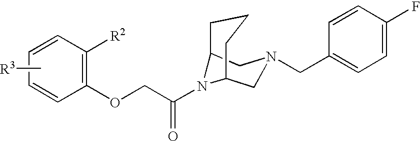

| Example | R³ | R² |
|---|---|---|
| 88 | 4-Cl | SO₂NH₂ |
| 89 | 4-Cl | CO₂CH₃ |
| 90 | 4-Cl | NHSO₂CH₃ |
| 91 | 3-Cl | NO₂ |
| 92 | 4-Cl | CH₂CO₂CH₂CH₃ |

### EXAMPLE 93

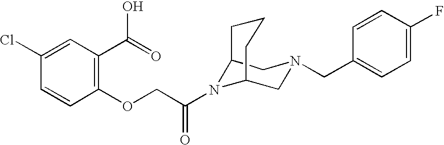

The title compound for Example 93 is prepared by a method analogous to that described in Example 20.

### EXAMPLE 94

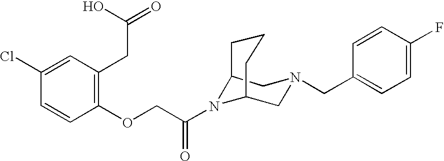

The title compound for Example 94 is prepared by a method analogous to that described in Example 20.

### EXAMPLE 95

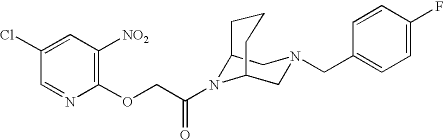

The title compound for Example 95 is prepared by a method analogous to that described in Example 15.

### EXAMPLE 96

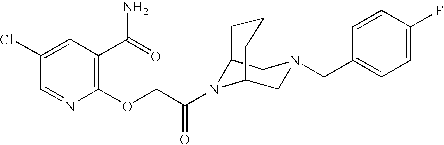

The title compound for Example 96 is prepared by a method analogous to that described in Example 15.

### EXAMPLE 97

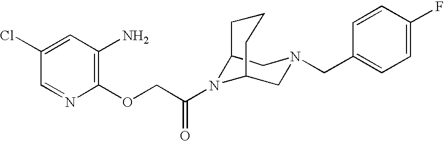

The title compound for Example 97 is prepared by a method analogous to that described in Example 42.

### EXAMPLE 98

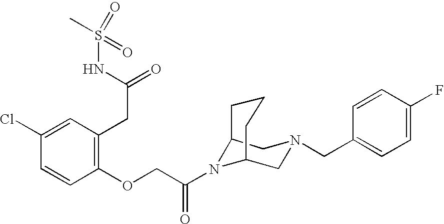

The title compound for Example 98 is prepared by a method analogous to that described in Example 30.

### EXAMPLE 99

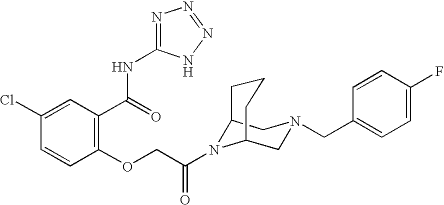

The title compound for Example 98 is prepared by a method analogous to that described in Example 30.

1. A compound of the formula 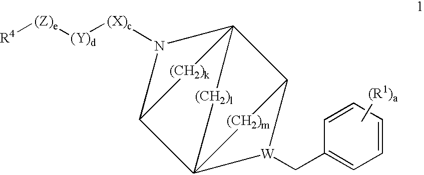
or the pharmaceutically acceptable salt and pro-drugs thereof; wherein a is 1, 2, 3, 4 or 5;
c is 0 or 1;
d is 1,2,3,4or 5;
k is 0, 1, 2, 3 or 4; l is 0, 1, 2, 3 or 4; m is 0, 1, 2, 3, or 4; k, l and m cannot all be 0 and if m and/or k are not 0, then I must be 0.;
W is CH or N;
X is C(O), C(S) or CH_{2;}
Y is CH_{2;}
Z is oxygen, NR⁹ or CR¹¹R¹²;
each R¹ is independently selected from hydrogen, hydroxy, hydroxysulfonyl, halo, (C₁-C₆)alkyl, mercapto, mercapto(C₁-C₆)alkyl, (C₁-C₆)alkylthio, (C₁-C₆)alkylsulfinyl, (C₁-C₆)alkylsufonyl, (C₁-C₆)alkylthio(C₁-C₆)alkyl, (C₁-C₆)alkylsulfinyl(C₁-C₆)alkyl, (C₁-C₆)alkylsulfonyl(C₁-C₆)alkyl, (C₁-C₆)alkoxy, (C₆-C₁₀)aryloxy, halo(C₁-C₆)alkyl, trifluoromethyl, formyl, formyl(C₁-C₆)alkyl, nitro, nitroso, cyano, (C₆-C₁₀)aryl(C₁-C₆)alkoxy, halo(C₁-C₆)alkoxy, trifluoromethoxy, (C₃-C₇)cycloalkyl, (C₃-C₇)cycloalkyl(C₁-C₆)alkyl, hydroxy(C₃-C₇)cycloalkyl(C₁-C₆)alkyl, (C₃-C₇)cycloalkylamino, (C₃-C₇)cycloalkylamino(C₁-C₆)alkyl, ((C₃-C₇)cycloalkyl)((C₁-C₆)alkyl)amino, ((C₃-C₇)cycloalkyl(C₁-C₆)alkyl)amino(C₁-C₆)alkyl, cyano(C₁-C₆)alkyl, (C₂-C₇)alkenyl, (C₂-C₇)alkynyl, (C₆-C₁₀)aryl, (C₆-C₁₀)aryl(C-C₆)alkyl, (C₆-C₁₀)aryl(C₂-C₆)alkenyl, hydroxy(C₁-C₆)alkyl, hydroxy(C₆-C₁₀)aryl (C₁-C₆)alkyl, hydroxy(C₁-C₆)alkylthio(C₁-C₆)alkyl, hydroxy(C₂-C₆)alkenyl, hydroxy(C₂-C₆)alkynyl, (C₁-C₆)alkoxy(C₁-C₆)alkyl, (C₁-C₆)alkoxy(C₆-C₁₀)aryl(C₁-C₆)alkyl, (C₆-C₁₀)aryloxy(C₁-C₆)alkyl, (C₆-C₁₀)aryl(C₁-C₆)alkoxy(C₁-C₆)alkyl, amino, (C₁-C₆)alkylamino, ((C₁-C₆)alkyl)₂amino, (C₆-C₁₀)arylamino, (C₆-C₁₀)aryl(C₁-C₆)alkylamino, amino(C₁-C₆)alkyl, (C₁-C₆)alkylamino(C₁-C₆)alkyl, ((C₁-C₆)alkyl)₂amino(C₁-C₆)alkyl, hydroxy(C₁-C₆)alkylamino(C₁-C₆)alkyl, (C₆-C₁₀)arylamino(C₁-C₆)alkyl, (C₆-C₁₀)aryl (C₁-C₆)alkylamino(C₁-C₆)alkyl, (C₁-C₆)alkylcarbonylamino, ((C₁-C₆)alkylcarbonyl)((C₁-C₆)alkyl)amino, (C₁-C₆)alkylcarbonylamino(C₁-C₆)alkyl, ((C₁-C₆)alkylcarbonyl)((C₁-C₆)alkyl)amino(C₁-C₆)alkyl, (C₁-C₆)alkoxycarbonylamino, (C₁-C₆)alkoxycarbonyl)(C₁-C₆)alkylamino, (C₁-C₆)alkoxycarbonylamino(C₁-C₆)alkyl, (C₁-C₆)alkoxycarbnony)((C₁-C₆)alkyl)amino(C₁-C₆)alkyl, carboxy, (C₁-C₆)alkoxycarbonyl, (C₆-C₁₀)aryl(C₁-C₆)alkoxycarbonyl, (C₁-C₆)alkylcarbonyl, (C₁-C₆)alkylcarbonyl(C₁-C₆)alkyl, (C₆-C₁₀)arylcarbonyl, (C₆-C₁₀)arylcarbonyl(C₁-C₆)alkyl, (C₆-C₁₀)aryl(C₁-C₆)alkylcarbonyl, (C₆-C₁₀)aryl(C₁-C₆)alkycarbonyl(C₁-C₆)alkyl, carboxy(C₁-C₆)alkyl, (C₁-C₆)alkoxycarbonyl(C₁-C₆)alkyl, (C₆-C₁₀)aryl(C₁-C₆)alkoxycarbonyl(C₁-C₆)alkyl, (C₁-C₆)alkoxy(C₁-C₆)alkylcarbonyloxy(C₁-C₆)alkyl, aminocarbonyl, (C₁-C₆)alkylaminocarbonyl, ((C₁-C₆)alkyl)₂aminocarbonyl, (C₆-C₁₀)arylaminocarbonyl, (C₆-C₁₀)aryl(C₁-C₆)alkylaminocarbonyl, aminocarbonyl(C₁-C₆)alkyl, (C₁-C₆)alkylaminocarbonyl(C₁-C₆)alkyl, ((C₁-C₆)alkyl)₂aminocarbonyl(C₁-C₆)alkyl, (C₆-C₁₀)arylaminocarbonyl(C₁-C₆)alkyl, (C₁-C₆)alkylaminocarbonyl(C₁-C₆)alkyl, amidino, guanidino, ureido, (C₁-C₆)alkylureido, ((C₁-C₆)alkyl)₂ureido, ureido(C₁-C₆)alkyl, (C₁-C₆)alkylureido(C₁-C₆)alkyl, ((C₁-C₆)alkyl)₂ureido(C₁-C₆)alkyl, (C₂-C₉)heterocycloalkyl, (C₂-C₉)heteroaryl, (C₂-C₉)heterocycloalkyl(C₁-C₆)alkyl and (C₂-C₉)heteroaryl(C₁-C₆)alkyl;
R⁴ is (R⁵Q_{q})_{f}(C₆-C₁₀)aryl, (R⁵Q_{q})_{f}(C₃-C₁₀)cycloalkyl, (R⁵Q_{q})KC₂-C₉)heteroaryl, (R⁵Q_{q})KC₂-C₉)heterocycloalkyl,
wherein f is 0, 1,2,3,4 or 5;
Q is (C₁-C₆)alkyl;
q is 0 or 1;
R⁵ is independently selected from: (C₂-C₉)heterocycloalkylcarbonyl, (C₂-C₉)heteroarylcarbonyl, (C₂-C₉)heteroaryl(C₁-C₆)alkylaminocarbonyl, (C₂-C₉)heteroarylaminocarbonyl, (C₂-C₉)heterocycloalkyl(C₁-C₆)alkylaminocarbonyl, (C₁-C₆)alkylsulfonylaminocarbonyl, (C₁-C₆)alkylsulfonylamino(C₁-C₆)alkylaminocarbonyl, ureido(C₁-C₆)alkylaminocarbonyl, (C₁-C₆)alkylureido(C₁-C₆)alkylaminocarbonyl, ((C₁-C₆)alkyl)₂ureido(C₁-C₆)alkylaminocarbonyl, halo(C₁-C₆)alkylaminocarbonyl, (C₁-C₆)alkylcarbonylamino(C₁-C₆)alkylaminocarbonyl, hydroxy(C₁-C₆)alkylaminocarbonyl, aminosulfonyl(C₁-C₆)alkylaminocarbonyl, carboxy(C₁-C₆)alkylaminocarbonyl, (C₁-C₆)alkylaminosulfonyl(C₁-C₆)alkylaminocarbonyl, amino(C₁-C₆)alkylcarbonylamino, (C₁-C₆)alkylamino(C₁-C₆)alkylcarbonylamino, carboxy(C₁-C₆)alkylcarbonylamino, carboxy(C₁-C₆)alkoxycarbonylamino, ((C₁-C₆)alkyl)₂amino(C₁-C₆)alkylcarbonylamino, acetylamino(C₁-*C₆)alkylcarbonylamino, (acetyl)((C₁-C₆)alkyl)amino(C₁-C₆)alkylcarbonylamino, (C₁-C₆)alkylsulfonylamino(C₁-C₆)alkylcarbonylamino, cyanoguanidino(C₁-C₆)alkylcarbonylamino, (C₁-C₆)alkylcyanoguanidino(C₁-C₆)alkylcarbonylamino, ((C₁-C₆)alkyl)₂cyanoguanidino(C₁-C₆)alkylcarbonylamino, aminocarbonyl(C₁-C₆)alkylcarbonylamino, aminocarbonylamino(C₁-C₆)alkylcarbonylamino, (C₁-C₆)alkylaminocarbonylamino(C₁-C₆)alkylcarbonylamino, ((C₁-C₆)alkyl)₂aminocarbonylamino(C₁-C₆)alkylcarbonylamino, (C₂-C₉)heteroaryl(C₁-C₆)alkylcarbonylamino, (C₂-C₉)heterocycloalkyl(C₁-C₆)alkylcarbonylamino, aminosulfonyl(C₁-C₆)alkylcarbonylamino, hydroxy(C₁-C₆)alkylureido, amino(C₁-C₆)alkylureido, (C₁-C₆)alkylamino(C₁-C₆)alkylureido, ((C₁-C₆)alkyl)₂amino(C₁-C₆)alkylureido, (C₂-C₉)heterocycloalkyl (C₁-C₆)alkylureido, (C₂-C₉)heteroarylureido, (C₂-C₉)heteroaryl(C₁-C₆)alkylureido, (C₁-C₆)alkylsulfonylureido, aminosulfonyl(C₁-C₆)alkylureido, aminocarbonyl(C₁-C₆)alkylureido, (C₁-C₆)alkylaminocarbonyl(C₁-C₆)alkylureido, ((C₁-C₆)alkyl)₂aminocarbonyl(C₁-C₆)alkylureido, acetylamino(C₁-C₆)alkylureido, (acetyl)((C₁-C₆)alkyl)amino(C₁-C₆)alkylureido, carboxy(C₁-C₆)alkylureido, halo(C₁-C₆)alkylsulfonylamino, amino(C₁-C₆)alkylsulfonylamino, (C₁-C₆)alkylamino(C₁-C₆)alkylsulfonylamino, ((C₁-C₆)alkyl)₂amino(C₁-C₆)alkylsulfonylamino, acetylamino(C₁-C₆)alkylsulfonylamino, (acetyl)((C₁-C₆)alkyl)amino(C₁-C₆)alkylsulfonylamino, ureido(C₁-C₆)alkylsulfonylamino, (C₁-C₆)alkylureido(C₁-C₆)alkylsulfonylamino, ((C₁-C₆)alkyl)₂ureido(C₁-C₆)alkylsulfonylamino, (C₁-C₆)alkylsulfonylamino(C₁-C₆)alkylsulfonylamino, cyanoguanidino(C₁-C₆)alkylsulfonylamino, carboxy(C₁-C₆)alkylsulfonylamino, (C₁-C₆)alkylcyanoguanidino(C₁-C₆)alkylsulfonylamino, ((C₁-C₆)alkyl)₂cyanoguanidino(C₁-C₆)alkylsulfonylamino, aminocarbonyl(C₁-C₆)alkylsulfonylamino, (C₁-C₆)alkoxycarbonylamino(C₁-C₆)alkylsulfonylamino, aminosulfonylaminocarbonyl(C₁-C₆)alkylaminosuffonylaminocarbonyl, ((C₁-C₆)alkyl)₂aminosulfonylaminocarbonyl, (C₆-C₁₀)arylsulfonyl, (C₁-C₆)alkylaminosulfonylamino, ((C₁-C₆)alkyl)₂aminosulfonylamino, aminocarbonyl(C₁-C₆)alkylamino(C₁-C₆)alkylsulfonylamino, (C₂-C₉)heterocycloalkyloxycarbonylamino(C₁-C₆)alkylsulfonylamino, (C₂-C₉)heteroaryloxycarbonylamino(C₁-C₆)alkylsulfonylamino, cyanoguanidino, (C₁-C₆)alkylcyanoguanidino, ((C₁-C₆)alkyl)₂cyanoguanidino, (C₂-C₉)heterocycloalkylcyanoguanidino, (C₂-C₉)heterocycloalkyl (C₁-C₆)alkylcyanoguanidino, (C₂-C₉)heteroaryl(C₁-C₆)alkylcyanoguanidino, amino(C₁-C₆)alkylcyanoguanidino, (C₁-C₆)alkylamino(C₁-C₆)alkylcyanoguanidino, ((C₁-C₆)alkyl)₂amino(C₁-C₆)alkylcyanoguanidino, aminocarbonyl(C₁-C₆)alkylcyanoguanidino, carboxy(C₁-C₆)alkylcyanoguanidino; (C₁-C₆)alkylaminocarbonyl(C₁-C₆)alkylcyanoguanidino, ((C₁-C₆)alkyl)₂aminocarbonyl(C₁-C₆)alkylcyanoguanidino, hydroxy(C₁-C₆)alkylamino, aminocarbonyl(C₁-C₆)alkylamino, carboxy(C₁-C₆)alkylamino, (C₁-C₆)alkylsulfonylamino(C₁-C₆)alkylamino, (C₁-C₆)alkoxycarbonylamino(C₁-C₆)alkylamino, aminosulfonyl(C₁-C₆)alkylamino, (C₂-C₉)heteroaryl(C₁-C₆)alkylamino, acetylamino(C₁-C₆)alkylamino, (acetyl)((C₁-C₆)alkyl)amino(C₁-C₆)alkylamino, (C₂-C₉)heterocycloalkyl (C₁-C₆)alkylamino, ((C₁-C₆)alkyl)₂amino(C₁-C₆)alkylamino, (C₁-C₆)alkylamino(C₁-C₆)alkylamino,(C₁-C₆)alkoxy(C₁-C₆)alkylamino, (C₁-C₆)alkoxycarbonyl(C₁-C₆)alkylamino, cyano(C₁-C₆)alkylamino, (C₂-C₉)heterocycloalkyloxycarbonylamino(C₁-C₆)alkylamino, (C₂-C₉)heteroaryloxycarbonylamino(C₁-C₆)alkylamino, cyanoguanidino(C₁-C₆)alkylamino, (C₁-C₆)alkylcyanoguanidino(C₁-C₆)alkylamino, ((C₁-C₆)alkyl)₂cyanoguanidino(C₁-C₆)alkylamino, ureido(C₁-C₆)alkylamino, (C₁-C₆)alkylureido(C₁-C₆)alkylamino, ((C₁-C₆)alkyl)₂ureido(C₁-C₆)alkylamino, aminocarbonyloxy(C₁-C₆)alkylamino, hydroxy(C₁-C₆)alkylcarbonylamino, (C₁-C₆)alkylaminocarbonyl(C₁-C₆)alkylcarbonylamino, ((C₁-C₆)alkyl)₂aminocarbonyl(C₁-C₆)alkylcarbonylamino, (C₁-C₆)alkoxycarbonylamino(C₁-C₆)alkylcarbonylamino, aminosulfonyl(C₁-C₆)alkylcarbonylamino, hydroxy(C₁-C₆)alkylamino(C₁-C₆)alkylcarbonylamino, ((C₁-C₆)alkyl)₂amino(C₁-C₆)alkylamino(C₁-C₆)alkylcarbonylamino (C₁-C₆)alkylamino(C₁-C₆)alkylamino(C₁-C₆)alkylcarbonylamino, amino(C₁-C₆)alkylamino(C₁-C₆)alkylcarbonylamino, (C₁-C₆)alkoxy(C₁-C₆)alkylamino(C₁-C₆)alkylcarbonylamino, (C₂-C₉)heterocycloalkyloxycarbonylamino, (C₂-C₉)heteroarylcarbonylamino(C₁-C₆)alkylcarbonylamino, (C₂-C₉)heteroarylcarbonylamino, (C₂-C₉)heterocycloalkylcarbonylamino, (C₂-C₉)heteroaryl(C₁-C₆)alkylcarbonylamino, (C₂-C₉)heterocycloalkyl(C₁-C₆)alkylcarbonylamino, (C₂-C₉)heterocycloalkylcarbonylamino(C₁-C₆)alkylcarbonylamino, cyano(C₁-C₆)alkylcarbonylamino, (C₁-C₆)alkylsulfonylamino(C₁-C₆)alkylaminocarbonylamino, (C₁-C₆)alkoxycarbonylamino(C₁-C₆)alkylaminocarbonylamino, (C₂-C₉)heterocycloalkyloxycarbonylamino(C₁-C₆)alkylaminocarbonylamino, (C₂-C₉)heteroaryloxycarbonylamino(C₁-C₆)alkylaminocarbonylaminol, ureido(C₁-C₆)alkylureido, (C₁-C₆)alkylureido(C₁-C₆)alkylureido, ((C₁-C₆)alkyl)₂ureido(C₁-C₆)alkylureido, cyanoguanidino(C₁-C₆)alkylureido, (C₂-C₉)heteroaryl(cyanoguanidino), aminosulfonyl, amino(C₁-C₆)alkylsulfonyl, (C₁-C₆)alkylamino(C₁-C₆)alkylsulfonyl, ((C₁-C₆)alkyl)₂amino(C₁-C₆)alkylsulfonyl, (C₁-C₆)alkylaminosulfonyl, ((C₁-C₆)alkyl)₂aminosulfonyl, (C₂-C₉)heterocycloalkylsulfonyl, amino(C₁-C₆)alkylaminosulfonyl, (C₁-C₆)alkylamino(C₁-C₆)alkylaminosulfonyl, ((C₁-C₆)alkyl)₂amino(C₁-C₆)alkylaminosulfonyl, (C₂C₉)heteroarylaminosulfonyl, hydroxy(C₁-C₆)alkylaminosulfonyl, (C₁-C₆)alkoxy(C₁-C₆)alkylaminosulfonyl, ureido(C₁-C₆)alkylaminosulfonyl, (C₁-C₆)alkylureido(C₁-C₆)alkylaminosulfonyl, ((C₁-C₆)alkyl)₂ureido(C₁-C₆)alkylaminosulfonyl, (C₁-C₆)alkylsulfonylamino(C₁-C₆)alkylaminosulfonyl, (C₁-C₆)alkoxycarbonylamino(C₁-C₆)alkylaminosulfonyl, (C₂-C₉)heterocycloalkyloxycarbonylamino(C₁-C₆)alkylaminosulfonyl, (C₂-C₉)heteroaryloxycarbonylamino(C₁-C₆)alkylaminosulfonyl, aminocarbonyl(C₁-C₆)alkylaminosulfonyl, cyanoguanidino(C₁-C₆)alkylaminosulfonyl, (C₂-C₉)heteroarylaminosulfonyl, (C₂-C₉)heteroaryl (C₁-C₆)alkylaminosulfonyl, (C₂-C₉)heterocycloalkylaminosulfonyl, (C₁-C₆)alkylcarbonylaminosulfonyl, halo(C₁-C₆)alkylcarbonylaminosulfonyl, (C₁-C₆)alkoxycarbonylaminosulfonyl, ureidosulfonyl, (C₁-C₆)alkylureidosulfonyl, ((C₁-C₆)alkyl)₂ureidosulfonyl, hydrogen, hydroxy, hydroxysulfonyl, halo, mercapto, (C₁-C₆)alkylthio, (C₁-C₆)alkylsulfinyl, (C₁-C₆)alkylsulfonyl, carboxy(C₁-C₆)alkylsulfonyl, (C₆-C₁₀)arylsulfonyl, (C₂-C₉)heteroarylsulfonyl, (C₁-C₆)alkoxy, hydroxy(C₁-C₆)alkoxy, (C₆-C₁₀)aryloxy, trifluoro(C₁-C₆)alkyl, formyl, nitro, nitroso, cyano halo(C₁-C₆)alkoxy, trifluoro(C₁-C₆)alkoxy, amino(C₁-C₆)alkoxy, (C₃-C₁₀)cycloalkylhydroxy(C₃-C₁₀)cycloalkyl (C₃-C₁₀)cycloalkylamino(C₂-C₆)alkenyl, (C₂-C₆)alkynyl, (C₆-C₁₀)aryl, (C₆-C₁₀)aryl(C₂-C₆)alkenyl, hydroxy(C₆-C₁₀)aryl, ((C₁-C₆)alkylamino)(C₆-C₁₀)aryl, hydroxy(C₁-C₆)alkylthio, hydroxy(C₂-C₆)alkenyl, hydroxy(C₂-C₆)alkynyl, (C₁-C₆)alkoxy(C₆-C₁₀)aryl, (C₆-C₁₀)aryl(C₁-C₆)alkoxy, amino, (C₁-C₆)alkylamino, ((C₁-C₆)alkyl)₂amino, (C₆-C₁₀)arylamino, (C₆-C₁₀)aryl(C₁-C₆)alkylamino, amino(C₁-C₆)alkylamino, (C₂-C₉)heterocycloalkylamino, (C₂-C₉)heteroarylamino, (C₂-C₉)heteroaryl(C₁-C₆)alkylamino, , (C₂-C₉)heterocycloalkyl(C₁-C₆)alkylamino, (C₃-C₁₀)cycloalkyl(C₁-C₆)alkyl)amino, (C₁-C₆)alkylcarbonylamino, (C₁-C₆)alkoxycarbonylamino, (C₂-C₆)alkenylcarbonylamino, (C₃-C₁₀)cycloalkylcarbonylamino, (C₆-C₁₀)arylcarbonylamino, (C₂-C₉)heterocycloalkylcarbonylamino, (C₂-C₉)heteroaryloxycarbonylamino, (C₂-C₉)heterocycloalkoxycarbonylamino, halo(C₁-C₆)alkylcarbonylamino, (C₁-C₆)alkoxy(C₁-C₆)alkylcarbonylamino, (C₁-C₆)alkoxycarbonyl(C₁-C₆)alkylcarbonylamino, ((C₁-C₆)alkylcarbonyl)((C₁-C₆)alkyl)amino, ((C₁-C₆)alkoxycarbonyl)((C₁-C₆)alkyl)amino, (C₁-C₆)alkylsulfonylamino, ((C₁-C₆)alkylcarbonyl)((C₁-C₆)alkyl)amino, (C₃-C₁o)cycloalkyl(C₁-C₆)alkyl)amino, ((C₁-C₆)alkylsulfonyl)((C₁-C₆)alkyl)amino, (C₂-C₉)heteroarylsulfonylamino, (C₆-C₁₀)arylsulfonylamino, ((C₆-C₁₀)arylsulfonyl)((C₁-C₆)alkyl)amino, carboxy, (C₁-C₆)alkoxycarbonyl, (C₆-C₁₀)aryl(C₁-C₆)alkoxycarbonyl, (C₁-C₆)alkylcarbonyl, carboxy(C₁-C₆)alkylcarbonyl, amino(C₁-C₆)alkylcarbonyl, (C₁-C₆)alkylamino(C₁-C₆)alkylcarbonyl, ((C₁-C₆)alkyl)₂amino(C₁-C₆)alkylcarbonyl, (C₆-C₁₀)arylcarbonyl, (C₂-C₉)heteroaryl(C₁-C₆)alkylcarbonyl, (C₆-C₁₀)aryl(C₁-C₆)alkylcarbonyl, hydroxy(C₁-C₆)alkoxycarbonyl, (C₁-C₆)alkoxy(C₁-C₆)alkylcarbonyloxy, ((C₁-C₆)alkyl)₂aminocarbonyloxyaminocarbonyl, hydroxyaminocarbonyl, (C₁-C₆)alkylaminocarbonyl, ((C₁-C₆)alkyl)₂aminocarbonyl, (C₆-C₁₀)arylaminocarbonyl, (C₆-C₁₀)aryl(C₁-C₆)alkylaminocarbonyl, (aminocarbonyl(C₁-C₆)alkylaminocarbonyl, ((C₁-C₆)alkylaminocarbonyl(C₁-C₆)alkylaminocarbonyl, (carboxy(C₁-C₆)alkyl)aminocarbonyl, ((C₁-C₆)alkoxycarbonyl(C₁-C₆)alkylaminocarbonyl, (amino(C₁-C₆)alkyl)aminocarbonyl, (hydroxy(C₁-C₆)alkylaminocarbonylamidino, hydroxyamidino, guanidino, ureido, (C₁-C₆)alkylureido, (C₆-C₁₀)arylureido, ((C₆-C₁₀)aryl)₂ureido, (C₆-C₁₀)aryl(C₁-C₆)alkylureido, halo(C₁-C₆)alkylureido, ((C₁-C₆)alkyl)((C₆-C₁₀)aryl)ureido, ((C₁-C₆)alkyl)₂ureido, halo(C₁-C₆)alkylcarbonylureido, (halo(C₁-C₆)alkyl)((C₁-C₆)alkyl)ureido, ((C₁-C₆)alkoxycarbonyl(C₁-C₆)alkyl)ureido, glycinamido, (C₁-C₆)alkylglycinamido, aminocarbonylglycinamido, (C₁-C₆)alkoxy(C₁-C₆)alkylcarbonylglycinamido, (aminocarbonyl)((C₁-C₆)alkyl)glycinamido, ((C₁-C₆)alkoxycarbonyl(C₁-C₆)alkylcarbonyl)((C₁-C₆)alkyl)glycinamido, ((C₁-C₆)alkoxycarbonylamino(C₁-C₆)alkylcarbonyl)glycinamido, (C₆-C₁₀)arylcarbonylglycinamido, ((C₆-C₁₀)arylcarbonyl)((C₁-C₆)alkyl)glycinamido, ((C₆-C₁₀)aryl(C₁-C₆)alkylaminocarbonyl)glycinamido, (C₆-C₁₀)aryl(C₁-C₆)alkylaminocarbonyl)((C₁-C₆)alkyl)glycinamido, (C₆-C₁₀)arylaminocarbonylglycinamido, ((C₆-C₁₀)arylaminocarbonyl)((C₁-C₆)alkyl)glycinamido, alaninamido, (C₁-C₆)alkylalaninamido, (C₂-C₉)heteroaryl, amino(C₂-C₉)heteroaryl, (C₁-C₆)alkylamino(C₂-C₉)heteroaryl, ((C₁-C₆)alkyl)₂amino(C₂-C₉)heteroaryl, (C₂-C₉)heteroaryloxy, (C₂-C₉)heterocycloalkyl, carboxy(C₁-C₆)alkoxy, (C₁-C₆)alkylsulfonylaminocarbonyl(C₁-C₆)alkoxy, (C₁-C₆)alkylsulfonylamino(C₁-C₆)alkoxy, (C₂-C₉)heteroaryl(C₁-C₆)alkoxy, carboxy(C₁-C₆)alkylamino(C₂-C₆)alkoxy, amino(C₂-C₆)alkoxy, (aminocarbonyl)(hydroxy)amino, (C₁-C₆)alkylamino(C₂-C₆)alkoxy, ((C₁-C₆)alkyl)₂amino(C₂-C₆)alkoxy, (C₁-C₆)alkylcarbonylamino(C₂-C₆)alkoxy, aminocarbonylamino(C₂-C₆)alkoxy, (C₁-C₆)alkylaminocarbonylamino(C₂-C₆)alkoxy, ((C₁-C₆)alkyl)₂aminocarbonylamino(C₂-C₆)alkoxy, amino(C₂-C₆)alkoxycarbonylamino, (C₁-C₆)alkylamino(C₂-C₆)alkoxycarbonylamino, ((C₁-C₆)alkyl)₂amino(C₂-C₆)alkoxycarbonylamino, C₂-C₉)heteroarylamino(C₂-C₆)alkoxy, barbituryl, (C₁-C₆)alkylcarbonylamino(C₁-C₆)alkylaminocarbonyl, amino(C₁-C₆)alkylcarbonylamino where the (C₁-C₆)alkyl is optionally substituted with one or two groups selected from hydrogen, amino, hydroxyl, (C₁-C₆)alkoxy, carboxy, further substituted (C₂-C₉)heteroaryl, (C₆-C₁₀)aryl, (C₂-C₉)heterocycloalkyl, and cycloalkyl, or the two groups together make up a carbocycle; and R¹⁹carbonylamino where R¹⁹ is a nitrogen containing (C₂-C₉)heterocycloalkyl which is optionally substituted further with one or two groups selected from (C₁-C₆)alkyl, (C₂-C₆)alkoxy and hydroxy;
R⁹ is selected from the group consisting of hydrogen, (C₁-C₆)alkyl, (C₆-C₁₀)aryl, (C₆-C₁₀)aryl(C₁-C₆)alkyl, (C₁-C₆)alkylcarbonyl, (C₁-C₆)alkylcarbonyl(C₁-C₆)alkyl, (C₆-C₁₀)aryl(C₁-C₆)alkylcarbonyl, (C₆-C₁₀)aryl(C₁-C₆)alkylcarbonyl(C₁-C₆)alkyl, aminocarbonyl, (C₁-*C₆)alkylaminocarbonyl, ((C₁-C₆)alkyl)₂aminocarbonyl and (C₁-C₆)alkoxycarbonyl; and
R¹¹ and R¹² are each independently selected from the group consisting of hydrogen, (C₁-C₆)alkyl, (C₆-C₁₀)aryl, (C₆-C₁₀)aryl(C₁-C₆)alkyl, hydroxy, (C₁-C₆)alkoxy, hydroxy(C₁-C₆)alkyl, (C₁-C₆)alkoxy(C₁-C₆)alkyl, amino, (C₁-C₆)alkylamino, ((C₁-C₆)alkyl)₂amino, (C₁-C₆)alkylcarbonylamino, (C₃-CS)cycloalkylcarbonylamino, (C₃-C₈)cycloalkyl(C₁-C₆)alkylcarbonylamino, (C₁-C₆)alkoxycarbonylamino, (C₁-C₆)alkylsulfonylamino, (C₆-C₁₀)arylcarbonylamino, (C₁-C₆)alkoxycarbonyl(C₁-C₆)alkylcarbonylamino, (C₆-C₁₀)aryl(C₁-C₆)alkylcarbonylamino, ((C₆-C₁₀)aryl(C₁-C₆)alkylcarbonyl)((C₁-C₆)alkyl)amino, (C₁-C₆)alkylcarbonylamino(C₁-C₆)alkyl, (C₃-C₈)cycloalkylcarbonylamino(C₁-C₆)alkyl, (C₁-C₆)alkoxycarbonylamino(C₁-C₆)alkyl, (C₂-C₉)heterocycloalkylcarbonylamino(C₁-C₆)alkyl, (C₆-C₁₀)aryl (C₁-C₆)alkylcarbonylamino(C₁-C₆)alkyl, (C₂-C₉)heteroarylcarbonylamino(C₁-C₆)alkyl, (C₆-C₁₀)arylsulfonylamino, (C₁-C₆)alkylsulfonylamino(C₁-C₆)alkyl, aminocarbonylamino, (C₁-C₆)alkylaminocarbonylamino, halo(C₁-C₆)alkylaminocarbonylamino, ((C₁-C₆)alkyl)₂aminocarbonylamino, aminocarbonylamino(C₁-C₆)alkyl, (C₁-C₆)alkylaminocarbonylamino(C₁-C₆)alkyl, ((C₁-C₆)alkyl)₂aminocarbonylamino(C₁-C₆)alkyl, halo(C₁-C₆)alkylaminocarbonylamino(C₁-C₆)alkyl, amino(C₁-C₆)alkyl, (C₁-C₆)alkylamino(C₁-C₆)alkyl, ((C₁-C₆)alkyl)₂amino(C₁-C₆)alkyl, carboxy(C₁-C₆)alkyl, (C₁-C₆)alkoxycarbonyl(C₁-C₆)alkyl, aminocarbonyl(C₁-C₆)alkyl and (C₁-C₆)alkylaminocarbonyl(C₁-C₆)alkyl.

2. A compound according to claim 1, wherein R¹ is hydrogen, halo, cyano, nitro, trifluoromethyl, trifluoromethoxy, (C₁-C₆)alkyl, hydroxy or (C₁-C₆)alkylcarbonyloxy.

3. A compound according to claim 1, wherein c is 1; X is C(O) or CH₂; d is 1; and Z is oxygen, NH, (C₁-C₆)alkyl, or CR R¹¹R¹².

4. A compound according to claim 1, wherein R⁴ is (R⁵)_{f}(C₆-C₁₀)aryl or (R⁵)_{f}(C₂-C₉) heteroaryl, wherein f is 1 or 2.

5. A compound according to claim 1, wherein c is 1; X is C(O); d is 1; Z is oxygen or (C₁-C₆)alkyl; W is nitrogen or CH; and 1, m and k are zero, zero and 2 or 3 respectively, or k, I, and m are zero, zero and 2 or 3 respectively.

6. A compound according to claim 1, wherein R⁴ is phenyl, Q is (C₁-C₆)alkyl, q is 0 or 1, and at least one R⁵ is selected from: (C₂-C₉)heteroarylaminocarbonyl, (C₂-C₉)heteroarylcarbonylamino, (C₁-C₆)alkylsulfonylaminocarbonyl, aminosulfonylaminocarbonyl, carboxy(C₁-C₆)alkylcyanoguanidino, carboxy, (C₂-C₉)heteroarylamino, (C₂-C₉)heteroarylsulfonyl, (C₂-C₉)heteroaryl (C₂-C₉)heteroaryloxy, (C₂-C₉)heteroarylcarbonyl, (C₂-C₉)heteroaryl(C₁-C₆)alkylcarbonyl, carboxy(C₁-C₆)alkylaminocarbonylamino, (C₂-C₉)heteroarylaminocarbonylamino, carboxy(C₁-C₆)alkylcarbonylamino, (C₂-C₉)heteroaryl (C₁-C₆)alkylamino, carboxy(C₁-C₆)alkylaminocarbonyl, carboxy(C₁-C₆)alkylsulfonylamino, (C₂-C₉)heteroarylaminosulfonyl, carboxy(C₁-C₆)alkylsulfonyl, carboxy(C₁-C₆)alkylamino, carboxy(C₁-C₆)alkylcarbonyl, carboxy(C₁-C₆)alkoxy, carboxy(C₁-C₆)alkoxycarbonylamino, hydroxyaminocarbonyl, (C₁-C₆)alkylsulfonylaminocarbonyl(C₁-C₆)alkoxy, (C₂-C₉)heteroaryl(C₁-C₆)alkoxy, carboxy(C₁-C₆)alkylamino(C₂-C₆)alkoxy, (C₂-C₉)heteroarylamino(C₂-C₆)alkoxy, amino(C₁-C₆)alkylcarbonyl, (C₁-C₆)alkylamino(C₁-C₆)alkylcarbonyl, ((C₁-C₆)alkyl)₂amino(C₁-C₆)alkylcarbonyl, amino(C₁-C₆)alkylcarbonylamino, (C₁-C₆)alkylamino(C₁-C₆)alkylcarbonylamino, ((C₁-C₆)alkyl)₂amino(C₁-C₆)alkylcarbonylamino, amino(C₁-C₆)alkylureido, (C₁-C₆)alkylamino(C₁-C₆)alkylureido, ((C₁-C₆)alkyl)₂amino(C₁-C₆)alkylureido, amino(C₁-C₆)alkylsulfonylamino, (C₁-C₆)alkylamino(C₁-C₆)alkylsulfonylamino, ((C₁-C₆)alkyl)₂amino(C₁-C₆)alkylsulfonylamino, amino(C₁-C₆)alkylsulfonyl, (C₁-C₆)alkylamino(C₁-C₆)alkylsuffonyl, ((C₁-C₆)alkyl)₂amino(C₁-C₆)alkylsulfonyl, amino(C₁-C₆)alkylcyanoguanidino, (C₁-C₆)alkylamino(C₁-C₆)alkylcyanoguanidino, ((C₁-C₆)alkyl)₂amino(C₁-C₆)alkylcyanoguanidino, amino(C₁-C₆)alkylaminosulfonyl, (C₁-C₆)alkylamino(C₁-C₆)alkylaminosulfonyl, ((C₁-C₆)alkyl)₂amino(C₁-C₆)alkylaminosulfonyl, ((C₁-C₆)alkylamino)(C₆-C₁₀)aryl(C₁-C₆)alkyl, amino, amino(C₁-C₆)alkoxy, amino(C₁-C₆)alkoxycarbonylamino, (C₁-C₆)alkylamino, ((C₁-C₆)alkyl)₂amino, (C₆-C₁₀)arylamino, (C₆-C₁₀)aryl(C₁-C₆)alkylamino, amino(C₁-C₆)alkylamino, (C₂-C₉)heterocycloalkylamino, (C₂-C₉)heteroarylamino, (C₃-C₁₀)cycloalkyl(C₁-C₆)alkyl)amino, (amino(C₁-C₆)alkyl)aminocarbonyl, glycinamido, (C₁-C₆)alkylglycinamido, alaninamido, (C₁-C₆)alkylalaninamido, ((C₁-C₆)alkyl)2 amino(C₁-C₆)alkylcarbonylamino, halo, (C₁-C₆)alkoxy, (C₁-C₆)alkyl, halo(C₁-C₆)alkyl, aminocarbonyl(C₁-C₆)alkylureido, (C₁-C₆)alkylcarbonyl, (C₁-C₆)alkylsulfonylamino, (C₁-C₆)alkylsulfonylamino(C₁-C₆)alkylaminocarbonyl, aminosulfonyl, aminocarbonyl, ureido(C₁-C₆)alkylaminocarbonyl, aminocarbonyl (C₁-C₆)alkyaminocarbonyl, aminocarbonyl(C₁-C₆)alkylcarbonylamino, ureido(C₁-C₆)alkylcarbonylamino, (C₁-C₆)alkylcarbonylamino(C₁-C₆)alkylcarbonylamino, (C₁-C₆)alkylcarbonylamino(C₁-C₆)alkylaminocarbonylamino, ureido(C₁-C₆)alkylcarbonylamino, ureido, halo(C₁-C₆)alkylsulfonylamino, (C₁-C₆)alkylcarbonylamino(C₁-C₆)alkylaminocarbonyl.

7. A compound according to claim 1, wherein R⁴ is pyridyl, Q is (C₁-C₆)alkyl, q is 0 or 1, and at least one R⁵ is selected from: (C₂-C₉)heteroarylaminocarbonyl, (C₂-C₉)heteroarylcarbonylamino, (C₁-C₆)alkylsulfonylaminocarbonyl, aminosulfonylaminocarbonyl, carboxy(C₁-C₆)alkylcyanoguanidino, carboxy, (C₂-C₉)heteroarylamino, (C₂-C₉)heteroarylsulfonyl, (C₂-C₉)heteroaryl (C₂-C₉)heteroaryloxy, (C₂-C₉)heteroarylcarbonyl, (C₂-C₉)heteroaryl(C₁-C₆)alkylcarbonyl, carboxy(C₁-C₆)alkylaminocarbonylamino, (C₂-C₉)heteroarylaminocarbonylamino, carboxy(C₁-C₆)alkylcarbonylamino, (C₂-C₉)heteroaryl(C₁-C₆)alkylamino, carboxy(C₁-C₆)alkylaminocarbonyl, carboxy(C₁-C₆)alkylsulfonylamino, (C₂-C₉)heteroarylaminosulfonyl, carboxy(C₁-C₆)alkylsulfonyl, carboxy(C₁-C₆)alkylamino, carboxy(C₁-C₆)alkylcarbonyl, carboxy(C₁-C₆)alkoxy, carboxy(C₁-C₆)alkoxycarbonylamino, hydroxyaminocarbonyl, (C₁-C₆)alkylsulfonylaminocarbonyl(C₁-C₆)alkoxy, (C₂-C₉)heteroaryl(C₁-C₆)alkoxy, carboxy(C₁-C₆)alkylamino(C₂-C₆)alkoxy, (C₂-C₉)heteroarylamino(C₂-C₆)alkoxy, amino(C₁-C₆)alkylcarbonyl, (C₁-C₆)alkylamino(C₁-C₆)alkylcarbonyl, ((C₁-C₆)alkyl)₂amino(C₁-C₆)alkylcarbonyl, amino(C₁-C₆)alkylcarbonylamino, (C₁-C₆)alkylamino(C₁-C₆)alkylcarbonylamino, ((C₁-C₆)alkyl)₂amino(C₁-C₆)alkylcarbonylamino, amino(C₁-C₆)alkylureido, (C₁-C₆)alkylamino(C₁-C₆)alkylureido, ((C₁-C₆)alkyl)₂amino(C₁-C₆)alkylureido, amino(C₁-C₆)alkylsulfonylamino, (C₁-C₆)alkylamino(C₁-C₆)alkylsulfonylamino, ((C₁-C₆)alkyl)₂amino(C₁-C₆)alkylsulfonylamino, amino(C₁-C₆)alkylsulfonyl, (C₁-C₆)alkylamino(C₁-C₆)alkylsulfonyl, ((C₁-C₆)alkyl)₂amino(C₁-C₆)alkylsulfonyl, amino(C₁-C₆)alkylcyanoguanidino, (C₁-C₆)alkylamino(C₁-C₆)alkylcyanoguanidino, ((C₁-C₆)alkyl)₂amino(C₁-C₆)alkylcyanoguanidino, amino(C₁-C₆)alkylaminosulfonyl, (C₁-C₆)alkylamino(C₁-C₆)alkylaminosulfonyl, ((C₁-C₆)alkyl)₂amino(C₁-C₆)alkylaminosulfonyl, ((C₁-C₆)alkylamino)(C₆-C₁₀)aryl(C₁-C₆)alkyl, amino, amino(C₁-C₆)alkoxy, amino(C₁-C₆)alkoxycarbonylamino, (C₁-C₆)alkylamino, ((C₁-C₆)alkyl)₂amino, (C₆-C₁₀)arylamino, (C₆-C₁₀)aryl(C₁-C₆)alkylamino, amino(C₁-C₆)alkylamino, (C₂-C₉)heterocycloalkylamino, (C₂-C₉)heteroarylamino, (C₃-C₁₀)cycloalkyl(C₁-C₆)alkyl)amino, (amino(C₁-C₆)alkyl)aminocarbonyl, glycinamido, (C₁-C₆)alkylglycinamido, alaninamido, (C₁-C₆)alkylalaninamido, ((C₁-C₆)alkyl)₂ amino(C₁-C₆)alkylcarbonylamino, aminocarbonyl(C₁-C₆)alkylureido, (C₁-C₆)alkylcarbonyl, (C₁-C₆)alkylsulfonylamino, (C₁-C₆)alkylsulfonylamino(C₁-C₆)alkylaminocarbonyl, aminosulfonyl, aminocarbonyl, ureido(C₁-C₆)alkylaminocarbonyl, aminocarbonyl(C₁-C₆)alkyaminocarbonyl, aminocarbonyl(C₁-C₆)alkylcarbonylamino, ureido(C₁-C₆)alkylcarbonylamino, (C₁-C₆)alkylcarbonylamino(C₁-C₆)alkylcarbonylamino, (C₁-C₆)alkylcarbonylamino(C₁-C₆)alkylaminocarbonylamino, ureido(C₁-C₆)alkylcarbonylamino, ureido, halo(C₁-C₆)alkylsulfonylamino, (C₁-C₆)alkylcarbonylamino(C₁-C₆)alkylaminocarbonyl.

8. Salts of a compound according to claim 1, where pharmaceutically acceptable counter-ions for acidic compounds are selected from alkali metal cations, alkaline earth metal cations ammonium or water-soluble amine addition salts, N-methylglucamine-(meglumine), the lower alkanolammonium and other base salts of pharmaceutically acceptable organic amines; and pharmaceutically acceptable salts selected from hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoatesalts.

9. A pharmaceutical composition for treating or preventing a disorder or condition selected from autoimmune diseases, rheumatoid arthritis, type I diabetes (recent onset), lupus, inflammatory bowel disease, optic neuritis, psoriasis, multiple sclerosis, polymyalgia rheumatica, uveitis, and vasculitis, acute and chronic inflammatory conditions osteoarthritis, adult Respiratory Distress Syndrome, Respiratory Distress Syndrome of infancy, ischemia reperfusion injury, glomerulonephritis, and chronic obstructive pulmonary disease (COPD) allergic conditions, asthma and atopic dermatitis, inflammation associated with infection, viral inflammation, influenza, hepatitis and Guillian-Barre, chronic bronchitis, chronic or acute tissue, cell, and solid organ transplant rejection, xeno-transplantation, atherosclerosis, restenosis, HIV infectivity (co-receptor usage), and granulomatous diseases, sarcoidosis, leprosy and tuberculosis, and sequelae associated with cancers, multiple myelomax; limiting the production of cytokines and/or TNF at inflammatory sites, as a consequence of decreasing cell infiltration; for treating diseases and/or congestive heart failure, linked to TNF and IL-1 and for treating pulmonary emphysema or dyspnea associated therewith, emphysema; HIV-1, HIV-2, HIV-3; cytomegalovirus (CMV), adenoviruses, Herpes viruses (Herpes zoster and Herpes simplex), for treating sequelae associated with infection where such infection induces production of detrimental inflammatory cytokines and/or TNF, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease, septic shock, cancer, trauma, and malaria, in a mammal, comprising an amount of a compound according to claim 1, or a pharmaceutically acceptable salt or pro-drug thereof, that is effective in treating or preventing such disorder or condition and a pharmaceutically acceptable carrier.

10. A pharmaceutical composition for treating or preventing a disorder or condition that can be treated or prevented by inhibiting chemokine binding to the receptor CCR1 in a ) mammal, comprising an amount of a compound according to claim 1, or a pharmaceutically acceptable salt or pro-drug thereof, effective in treating or preventing such disorder or condition and a pharmaceutically acceptable carrier.

11. A method for treating or preventing a disorder or condition selected from autoimmune diseases, rheumatoid arthritis, type I diabetes (recent onset), lupus, inflammatory bowel disease, optic neuritis, psoriasis, multiple sclerosis, polymyalgia rheumatica, uveitis, and vasculitis, acute and chronic inflammatory conditions osteoarthritis, adult Respiratory Distress Syndrome, Respiratory Distress Syndrome of infancy, ischemia reperfusion injury, glomerulonephritis, and chronic obstructive pulmonary disease (COPD) allergic conditions, asthma and atopic dermatitis, inflammation associated with infection, viral inflammation, influenza, hepatitis and Guillian-Barre, chronic bronchitis, chronic or acute tissue, cell, and solid organ transplant rejection, xeno-transplantation, atherosclerosis, restenosis, HIV infectivity (co-receptor usage), and granulomatous diseases, sarcoidosis, leprosy and tuberculosis, and sequelae associated with cancers, multiple myelomax; limiting the production of cytokines and/or TNF at inflammatory sites, as a consequence of decreasing cell infiltration; for treating diseases and/or congestive heart failure, linked to TNF and IL-1 and for treating pulmonary emphysema or dyspnea associated therewith, emphysema; HIV-1, HIV-2, HIV-3; cytomegalovirus (CMV), adenoviruses, Herpes viruses (Herpes zoster and Herpes simplex), for treating sequelae associated with infection where such infection induces production of detrimental inflammatory cytokines and/or TNF, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease, septic shock, cancer, trauma, and malaria, in a mammal, comprising administering to a mammal in need of such treatment or prevention an amount of a compound according to claim 1, or a pharmaceutically acceptable salt or pro-drug thereof, that is effective in treating or preventing such disorder or condition.

12. A method for treating or preventing a disorder or condition that can be treated or prevented by antagonizing the CCR1 receptor in a mammal, comprising administering to a mammal in need of such treatment or prevention an amount of a compound according to claim 1, or a pharmaceutically acceptable salt or pro-drug thereof, that is effective in treating or preventing such disorder or condition.

13. A pharmaceutical composition for treating or preventing a disorder or condition selected from autoimmune diseases, rheumatoid arthritis, type I diabetes (recent onset), lupus, inflammatory bowel disease, optic neuritis, psoriasis, multiple sclerosis, polymyalgia rheumatica, uveitis, and vasculitis, acute and chronic inflammatory conditions osteoarthritis, adult Respiratory Distress Syndrome, Respiratory Distress Syndrome of infancy, ischemia reperfusion injury, glomerulonephritis, and chronic obstructive pulmonary disease (COPD) allergic conditions, asthma and atopic dermatitis, inflammation associated with infection, viral inflammation, influenza, hepatitis and Guillian-Barre, chronic bronchitis, chronic or acute tissue, cell, and solid organ transplant rejection, xeno-transplantation, atherosclerosis, restenosis, HIV infectivity (co-receptor usage), and granulomatous diseases, sarcoidosis, leprosy and tuberculosis, and sequelae associated with cancers, multiple myelomax; limiting the production of cytokines and/or TNF at inflammatory sites, as a consequence of decreasing cell infiltration; for treating diseases and/or congestive heart failure, linked to TNF and IL-1 and for treating pulmonary emphysema or dyspnea associated therewith, emphysema; HIV-1, HIV-2, HIV-3; cytomegalovirus (CMV), adenoviruses, Herpes viruses (Herpes zoster and Herpes simplex), for treating sequelae associated with infection where such infection induces production of detrimental inflammatory cytokines and/or TNF, fungal meningitis, joint tissue damage, hyperplasia, pannus formation and bone resorption, psoriatic arthritis, hepatic failure, bacterial meningitis, Kawasaki syndrome, myocardial infarction, acute liver failure, lyme disease, septic shock, cancer, trauma, and malaria, in a mammal, comprising a CCR1 receptor antagonizing effective amount of a compound according to claim 1, or a pharmaceutically acceptable salt thereof, and a pharmaceutically acceptable carrier.

14. A pharmaceutical composition for treating or preventing a disorder or condition that can be treated or prevented by antagonizing the CCR1 receptor in a mammal, comprising a CCR1 receptor antagonizing effective amount of a compound according to claim 1, or a pharmaceutically acceptable salt or pro-drug thereof, and a pharmaceutically acceptable carrier.
